(19) United States
(12) Patent Application Publication
(10) Pub. No.: US 2025/0019715 A1
(43) Pub. Date: Jan. 16, 2025

(54) Ribosomal Biosynthesis Of Moroidin Peptides In Plants

(71) Applicant: Whitehead Institute for Biomedical Research, Cambridge, MA (US)

(72) Inventors: Roland D. Kersten, Arlington, MA (US); Jing-Ke Weng, Belmont, MA (US)

(21) Appl. No.: 18/712,830

(22) PCT Filed: Nov. 23, 2022
(86) PCT No.: PCT/US2022/080458

§ 371 Date: May 23, 2024

Related U.S. Application Data
(60) Provisional application No. 63/283,133, filed on Nov. 24, 2021.

(51) Int. Cl. C12N 15/82; C07K 7/54

(52) U.S. Cl. CPC C12N 15/8257; C07K 7/54

(57) ABSTRACT

An alternative route to moroidin-type bicyclic peptide biosynthesis is presented. Also included herein, it is reported that such moroidin-type bicyclic peptides are ribosomally synthesized and post-translationally modified peptides (RiPPs) in plants. Whereas D. moroides and C. argentea entail a previously uncharacterized DUF2775 family protein as candidate precursor peptides for moroidin biosynthesis, Japanese kerria (Kerria japonica) employs a BURP-domain protein as a precursor peptide similar to that of the recently reported lyciumin biosyntheti system. Disclosed herein are compositions and methods related to the biosynthesis of moroidin. In some embodiments of the disclosure, the moroidin peptides are synthetic. In other embodiments, the moroidin peptides are heterogenous. A skilled artisan will readily appreciate that based on the data disclosed herein that the present disclosure provides for the production of moroidins in transgenic host cells.

### RELATED APPLICATION

This application claims the benefit of U.S. Provisional Application No. 63/283,133, filed on Nov. 24, 2021. The entire teachings of the above application are incorporated herein by reference.

### INCORPORATION BY REFERENCE OF MATERIAL IN XML

This application incorporates by reference the Sequence Listing contained in the following eXtensible Markup Language (XML) file being submitted concurrently herewith:

File name: 03992067001.xml; created Nov. 23, 2022, 108,126 bytes in size.

### BACKGROUND

Moroidin is a bicyclic plant octapeptide with unusual tryptophan side-chain cross-links, originally isolated as a pain-causing agent from Dendrocnide moroides, an Australian stinging tree of the Urticaceae family. Moroidin and its structural analog celogentin C, derived from Celosia argentea of the Amaranthaceae family, are potent inhibitors of tubulin polymerization. However, low isolation yields from source plants and difficulty in organic synthesis hinder moroidin-based drug development.

### SUMMARY

Here, an alternative route to moroidin-type bicyclic peptide biosynthesis is presented. Also included herein, it is reported that such moroidin-type bicyclic peptides are ribosomally synthesized and post-translationally modified peptides (RiPPs) in plants. Whereas D. moroides and C. argentea entail a previously uncharacterized DUF2775 family protein as candidate precursor peptides for moroidin biosynthesis, Japanese kerria (Kerria japonica) employs a BURP-domain protein as a precursor peptide similar to that of the recently reported lyciumin biosynthetic system. The BURP domain is the moroidin cyclase that is suggested to install the indole-derived C—C and C—N bonds key to the moroidin bicyclic motif Based on these biosynthetic studies, new moroidin chemistry was discovered in legume, rose and amaranth plants by mining plant genomes and transcriptomes for moroidin precursor genes. These demonstrate the feasibility of producing diverse moroidins in transgenic tobacco plants, setting the stage for future development of moroidin-based therapeutics.

Described herein is a method of producing one or more moroidin cyclic peptides. In some embodiments, the method of producing one or more moroidin cyclic peptides can include providing a host cell comprising a transgene encoding a moroidin precursor peptide, or a biologically-active fragment thereof, wherein the moroidin precursor peptide, or biologically-active fragment thereof, comprises one or more core moroidin peptide domains; expressing the transgene in the host cell to thereby produce a moroidin precursor peptide, or biologically-active fragment thereof, wherein the moroidin precursor peptide, or biologically-active fragment thereof, is converted to one or more moroidin cyclic peptides in the host cell or wherein the moroidin precursor peptide, or biologically-active fragment thereof is isolated from the host cell and is then converted into a moroidin cyclic peptide in vitro using one or more enzymes such as an enzyme that cyclizes the moroidin precursor peptide; an endopeptidases; a glutamine cyclotransferases; an exopeptidases, or a combination thereof.

Described herein also is a method of generating a library of nucleic acids encoding moroidin precursor peptides, or biologically active fragments thereof. The method can include constructing a plurality of vectors, each vector comprising a nucleic acid encoding a different moroidin precursor peptide, or biologically-active fragment thereof, operably linked to a heterologous promoter for expression in a host cell. In some embodiments, the library can include at least at least hundreds of nucleic acids, e.g., at least 10³ nucleic acids, at least 10⁴ nucleic acids, at least 10⁵ nucleic acids, at least 10⁶ nucleic acids, or at least 10⁷ nucleic acids.

In some embodiments, the method of generating a library of nucleic acids can include introducing the plurality of vectors into host cells. In certain embodiments, the moroidin precursor peptide, or biologically-active fragments thereof, can be converted to one or more moroidin cyclic peptides in the host cell. In some embodiments, the host cell is a plant cell. In some embodiments, the plant cell is a Solanaceae family plant cell. In some embodiments, the plant cell is a Nicotiana genus plant cell, such as a Nicotiana benthamiana plant cell.

In some embodiments, the method can include isolating a moroidin cyclic peptide from the host cell. In some embodiments, the method can include assaying for an activity of interest either crude extract from the host cell or a moroidin peptide isolated from the host cell.

In some embodiments, the method of generating a library of nucleic acids can include introducing a nucleic acid encoding a moroidin peptide having an activity of interest into a second host cell. In some embodiments, the second host cell is a plant cell. In some embodiments, the plant cell is an Amaranthaceae family plant cell. In some embodiments, the plant cell is an Amaranthus genus plant cell, such as an Amaranthus hypochondriacus plant cell. In some embodiments, the plant cell is a Beta genus plant cell, such as a Beta vulgaris plant cell. In some embodiments, the plant cell is a Chenopodium genus plant cell, such as a Chenopodium quinoa plant cell. In some embodiments, the plant cell is a Fabaceae family plant cell. In some embodiments, the plant cell is a Glycine genus plant cell, such as a Glycine max plant cell. In some embodiments, the plant cell is a Medicago genus plant cell, such as a Medicago truncatula plant cell. In some embodiments, the plant cell is a Solanaceae family plant cell. In some embodiments, the plant cell is a Solanum genus plant cell, such as a Solanum melongena plant cell or a Solanum tuberosum plant cell. In some embodiments, the plant cell is a Nicotiana genus plant cell, such as a Nicotiana benthamiana plant cell. In some embodiments, the plant cell is a Capsicum genus plant cell, such as a Capsicum annuum plant cell.

Further described herein is a library that includes a plurality of nucleic acid molecules, each nucleic acid molecule including a nucleotide sequence encoding a moroidin precursor peptide, or a biologically-active fragment thereof. In some embodiments, the nucleotide sequence encoding a moroidin precursor peptide, or a biologically-active fragment thereof, is operably linked to a heterologous promoter in each nucleic acid molecule. In some embodiments, the nucleic acid molecules are complementary DNA (cDNA) molecules.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A shows moroidin structure. FIG. 1B shows LC-MS chemotyping of moroidin in leaf peptide extract of D. moroides and seed and flower peptide extracts of C. argentea. FIG. 1C shows candidate moroidin precursor peptide, CarMorA, derived from the de novo transcriptome of C. argentea flower tissue and candidate moroidin precursor peptide, DmoMorA, derived from the de novo transcriptome of D. moroides leaf tissue. Core peptides are highlighted with a box, SignalP40-predicted signal peptide is underlined, DUF2775-domain sequences are highlighted with shaded background.

FIG. 2A shows genome locus of predicted DUF2775 moroidin precursor genes in Amaranthus hypochondriacus and corresponding moroidin precursor peptide sequences. Different core peptides are highlighted, SignalP40-predicted signal peptides are underlined, DUF2775-domain sequences are highlighted with shaded background. FIG. 2B shows predicted structures of A. hypochondriacus moroidin peptides and the corresponding core peptides. FIG. 2C shows LC-MS-based moroidin peptide chemotyping of A. hypochondriacus and A. cruentus. Abbreviations: BPC—Base peak chromatogram.

FIG. 3A shows predicted moroidin precursor peptides from K. japonica and B. tomentosa resulted from mining plant transcriptomes of the 1 kp database. Predicted moroidin core peptides are highlighted with boxes, SignalP-predicted signal peptides are underlined, BURPdomain sequences are highlighted with shaded background. FIG. 3B shows predicted moroidin chemotypes of K. japonica and B. tomentosa. FIG. 3C shows LC-MS detection of predicted moroidin chemotypes in peptide extracts of K. japonica leaves and B. tomentosa seeds.

FIG. 4A shows LC-MS detection of moroidin from N. benthamiana leaves after transient expression of precursor gene KjaBURP for six days (Abbreviation: KjaBURP). FIG. 4B shows LC-MS detection of moroidin-[QLLVWRAH] (SEQ ID NO: 41) from N. benthamiana leaves after transient expression of precursor gene KjaBURP for six days (Abbreviation: KjaBURP). FIG. 4C shows Reconstitution of moroidin biosynthesis in N. benthamiana after transient co-expression of the N-terminal core peptide domain of KjaBURP (Abbreviation: KjaBURP-N) and a KjaBURP construct without core peptides (KjaBURP-no-core). FIG. 4D shows LC-MS detection of moroidin derivatives with N-terminal glutamines, N-terminal extensions and C-terminal extensions in peptide extracts of N. benthamiana leaves after transient expression of KjaBURP for six days. FIG. 4E shows proposed moroidin biosynthesis from precursor peptide KjaBURP based on N. benthamiana transient expression experiments.

FIG. 5A shows LC-MS detection of moroidin from peptide extracts of N. benthamiana leaves after transient expression of KjaBURP-[QLLVWRGH-lx] (SEQ ID NO: 35) for six days. FIG. 5B shows moroidin diversification via KjaBURP-[QLLVWRGH-lx](SEQ ID NO: 35) core peptide (R1-9) mutagenesis and transient expression in N. benthamiana. FIG. 5C shows quantitative chemotyping of moroidin from peptide extracts of N. benthamiana leaves after transient expression of KjaBURP, KjaBURP-[QLLVWRGH-lx] (SEQ ID NO: 35) or KjaBURPN+KjaBURP-no-core in comparison to peptide extracts of C. argentea flower. n=3 biological samples, error bars indicate ±1 σ (standard deviation).

FIG. 6 is table showing NMR analysis of moroidin from Celosia argentea var. cristata (600 MHz, DMSO-d6) [a] ¹³C NMR data of isolated moroidin in DMSO-d6. Chemical shift values were derived from ¹³C NMR analysis, HSQC analysis and HMBC analysis. [b] ¹H NMR data of isolated moroidin in DMSO-d6. Multiplicity m (s=singlet, d=doublet, t=triplet, dd=double doublet, dt=double triplet, m=multiplet), intensity int, coupling constants J in Hertz. Chemical shift values in ppm were derived from ¹H NMR analysis, ¹H-¹H-COSY analysis and 1H-1H-TOCSY analysis. [c] ¹H-¹H COSY correlations of isolated moroidin in DMSO-d6. [d]¹H-¹³C-HMBC correlations of isolated moroidin in DMSOd6. [e] NOESY correlations of isolated moroidin in DMSO-d6.

FIG. 7 is a table showing NMR analysis of [Asn9]-moroidin from C. argentea var. cristata (600 MHz, DMSO-d6) [a] ¹³C NMR data of isolated [Asn9]-moroidin in DMSO-d6. Values were derived from HSQC and HMBC analyses [b] ¹H NMR data of isolated [Asn9]-moroidin in DMSO-d6. Multiplicity m (s=singlet, d=doublet, t=triplet, dd=double doublet, dt=double triplet, m=multiplet), intensity int, coupling constants Jin Hertz. [c] ¹H-¹H COSY correlations from DQF-COSY analysis. [d] ¹H-¹³C HMBC correlations from HMBC analysis.

FIG. 8 is a table showing 1 kp database transcriptome mining of moroidin precursor peptides in terrestrial plants (Abbreviation: n/a—not available, X—any amino acid).

FIG. 9 is a table showing NMR analysis of celogentin C from N. benthamiana after transient expression of KjaBURP-[QLLVWPRH] (SEQ ID NO: 45) (600 MHz, DMSO-d6, 300 K) [a] ¹³C NMR data of isolated celogentin C in DMSO-d6. Chemical shift values were derived from ¹³C NMR analysis (FIG. 27). [b] ¹³C NMR data of isolated celogentin C in DMSO-d6 from Kobayashi, J., et al. 2001 J. Org. Chem. 66, 6626-6633. [c] ¹³C NMR data of synthetic celogentin C in DMSO-d6 from Ma, B., et al. 2009 J. Am. Chem. Soc. 132, 1159-1171. [d] ¹H NMR data of isolated celogentin C in DMSO-d6. Multiplicity m (s=singlet, d=doublet, t=triplet, dd=double doublet, m=multiplet, brs=broad singlet), intensity int, coupling constants Jin Hertz. Chemical shift values were derived from ¹H NMR analysis (FIG. 27), [e] ¹H NMR data of isolated celogentin C in DMSO-d6 from Kobayashi, J., et al. 2001 J. Org. Chem. 66, 6626-6633.

FIG. 10 shows moroidin derivatives, celogentins A-K, isolated from C. argentea. Celogentin A-C, celogentin D-J, celogentin K.

FIG. 11 shows ribosomal peptide natural products with tryptophan macrocyclizations.

FIGS. 12A-12B show candidate moroidin precursor transcripts identified by tblastn search of putative core peptide QLLVWRGH (SEQ ID NO: 59) in de novo transcriptome assemblies (Trinity (v2.4) or rnaSPAdes (v1.0) of C. argentea flower (FIG. 12A) and D. moroides leaf (FIG. 12B). FIG. 12C shows gene expression analysis of candidate moroidin precursors CarMorA and DmoMorA in de novo transcriptomes of C. argentea flower and D. moroides leaf, respectively.

FIG. 13A shows the structure of [Ala9]-moroidin. FIG. 13B shows the structure of [Ala9-Ala10]-moroidin.

FIG. 14A shows predicted AhyCelA and AhyMorA genes in A. hypochondriacus genome (v2.1). Introns and exons are highlighted with black boxes. FIG. 14B shows A. hypochondriacus gene cluster analysis. FIG. 14C shows cloned Amaranthus cruentus moroidin precursor peptide.

FIG. 15 shows predicted moroidin precursor peptides from Crossopetalum rhacoma (SRA: ERR2040328, Celastraceae), Bauhinia tomentosa (SRA: ERR706821, Fabaceae), Amaranthus tricolor (SRA: ERR2040205, Amaranthaceae) and Amaranthus retroflexus (SRA: ERR2040206, Amaranthaceae) from 1 kp database (rnaSPAdes-reassembled transcriptomes). Predicted moroidin core peptides are highlighted with boxes, BURP domain is underlined.

FIG. 16 shows KjaBURP constructs for co-expression analysis.

FIG. 17A shows characterized peptide analytes with bicyclic moroidin core structure. FIG. 17B shows tandem MS fragment ions derived from N-terminal glutamine including glutamine and pyroglutamate iminium ions and peptide ions with N-terminal pyroglutamate generated in situ during MS analysis. Corresponding pyroglutamate ions are indicated in MS/MS analyses by numbers in this Figure. FIG. 17C shows MS analysis of [Gln1]-moroidin chemotype in peptide extract of N. benthamiana leaves six days after infiltration with A. tumefaciens LBA4404 pEAQ-HT-KjaBURP. FIG. 17D shows MS analysis of [Gln1]-moroidin-[QLLVWRAH] (SEQ ID NO: 41) chemotype in peptide extract of N. benthamiana leaves six days after infiltration with A. tumefaciens LBA4404 pEAQ-HT-KjaBURP. FIG. 17E shows MS analysis of [AsnO-Gln1]-moroidin chemotype in peptide extract of N. benthamiana leaves six days after infiltration with A. tumefaciens LBA4404 pEAQ-HT-KjaBURP or with A. tumefaciens LBA4404 pEAQ-HT-KjaBURP-[QLLVWRGH-lx] (SEQ ID NO: 35). FIG. 17F shows MS analysis of [Gln1-Val9]-moroidin chemotype in peptide extract of N. benthamiana leaves six days after infiltration with A. tumefaciens LBA4404 pEAQ-HT-KjaBURP. FIG. 17G shows MS analysis of [Gln1-Val9]-moroidin-[QLLVWRAH] (SEQ ID NO: 41) chemotype in peptide extract of N. benthamiana leaves six days after infiltration with A. tumefaciens LBA4404 pEAQ-HT-KjaBURP. FIG. 17H shows MS analysis of [Val9]-moroidin chemotype in peptide extract of N. benthamiana leaves six days after infiltration with A. tumefaciens LBA4404 pEAQ-HT-KjaBURP. FIG. 17I shows MS analysis of [Val9]-moroidin-[QLLVWRAH] (SEQ ID NO: 41) chemotype in peptide extract of N. benthamiana leaves six days after infiltration with A. tumefaciens LBA4404 pEAQ-HT-KjaBURP.

FIG. 18 shows KjaBURP (SEQ ID NO: 34) precursor peptide with one moroidin core peptide.

FIG. 19A shows moroidin-[ALLVWRGH] (SEQ ID NO: 36) precursor peptide. FIG. 19B shows predicted moroidin-[ALLVWRGH] (SEQ ID NO: 36) chemotype.

FIG. 20A shows moroidin-[QALVWRGH] (SEQ ID NO: 37) precursor peptide. FIG. 20B shows putative moroidin-[QALVWRGH] (SEQ ID NO: 37) chemotype.

FIG. 21A shows moroidin-[QLAVWRGH] (SEQ ID NO: 38) precursor peptide. FIG. 21B shows putative moroidin-[QLAVWRGH] (SEQ ID NO: 38) chemotype.

FIG. 22A shows moroidin-[QLLAWRGH] (SEQ ID NO: 39) precursor peptide. FIG. 22B shows putative moroidin-[QLLAWRGH] (SEQ ID NO: 39) chemotype.

FIG. 23A shows moroidin-[QLLVWAGH] (SEQ ID NO: 40) precursor peptide. FIG. 23B shows putative moroidin-[QLLVWAGH] (SEQ ID NO: 40) chemotype.

FIG. 24A shows moroidin-[QLLVWRAH] (SEQ ID NO: 41) precursor peptide. FIG. 24B shows putative moroidin-[QLLVWRAH] (SEQ ID NO: 41) chemotype.

FIG. 25A shows moroidin-[QLLVWRH] (SEQ ID NO: 42) precursor peptide. FIG. 25B shows putative moroidin-[QLLVWRH] (SEQ ID NO: 42) chemotype.

FIG. 26A shows moroidin-[QLLVWRGGH] (SEQ ID NO: 43) precursor peptide.

FIG. 26B shows putative moroidin-[QLLVWRGGH] (SEQ ID NO: 43) chemotype.

FIG. 27 shows celogentin C precursor peptide (SEQ ID NO: 44).

### DETAILED DESCRIPTION

A description of example embodiments follows.

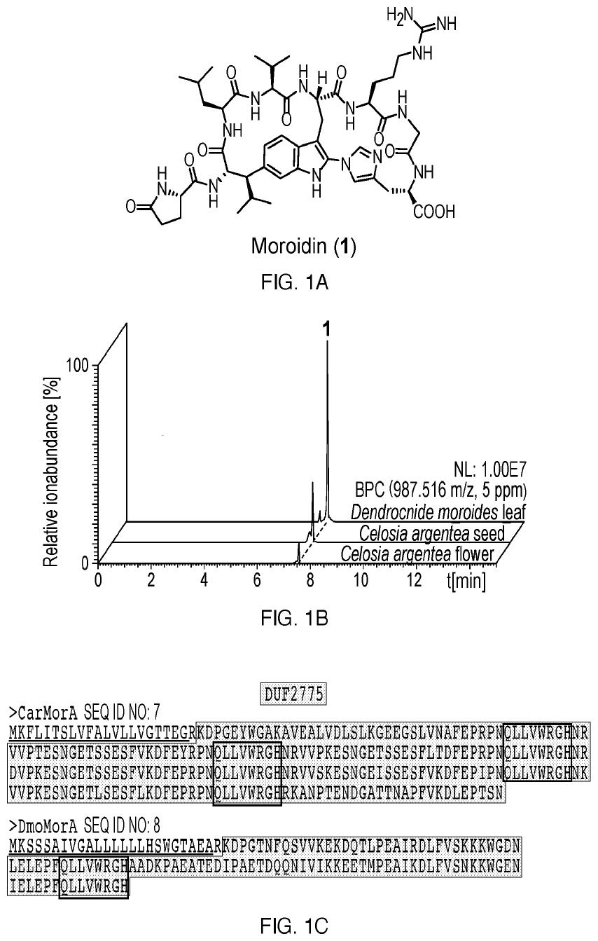

Natural toxins have provided important lead structures for therapeutics. The venom of Brazilian viper Bothrops jararaca led to the development of captopril, a drug for treating hypotension and heart failure, and the venom of cone snail Conus magnus inspired the chronic pain medication ziconotide. In the plant kingdom, Dendrocnide moroides or ‘gympie gympie’, a tree of the nettle family (Urticaceae) from the rainforests of East Australia, has been reported as one of the most painful plants. All aerial parts of the plant are covered with small trichomes, which can pierce the skin when the plant is touched, and cause a long-lasting pain sensation in humans for up to several weeks4. Due to its pain-causing activity, the plant has been investigated for the corresponding phytotoxins, and a peptide natural product called moroidin was isolated as one of the major active compounds (FIG. 1).

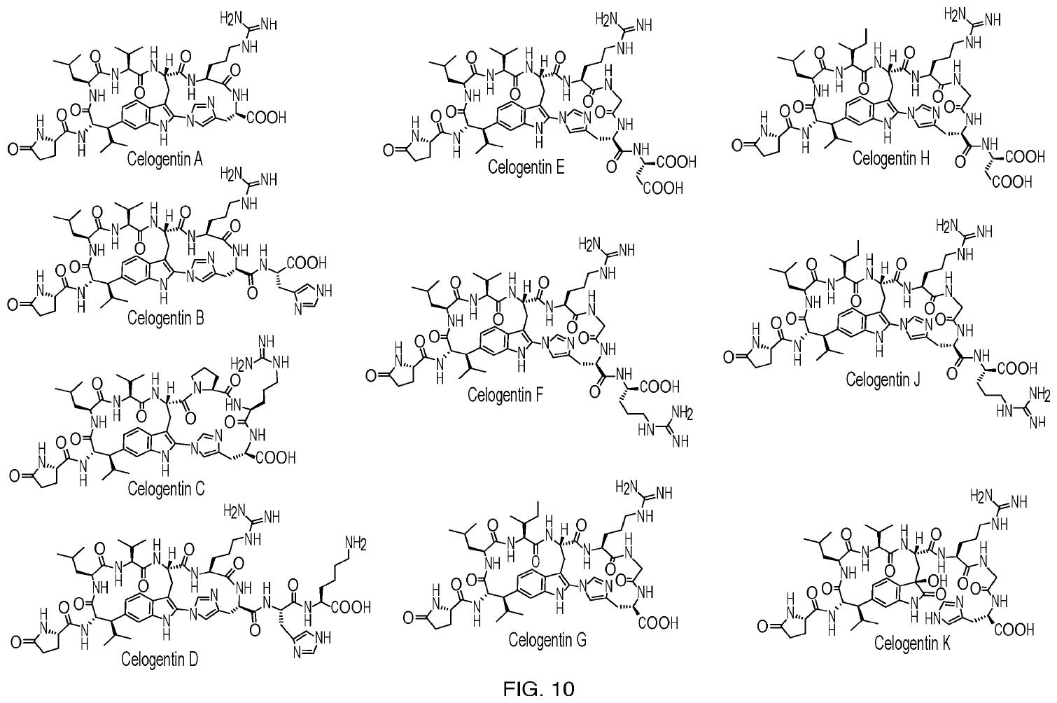

Moroidin is a bicyclic octapeptide, which is characterized by an N-terminal pyroglutamate and two side-chain macrocyclic linkages: (1) a C—C bond between the C6 of a tryptophan-indole at the fifth position and a β-carbon of a leucine at the second position and (2) a C—N bond between the C2 of the same tryptophan-indole and the N1 of a C-terminal histidine-imidazole (FIG. 1). Interestingly, moroidin and several structural derivatives called celogentins (FIG. 10) have also been isolated from the seeds of Celosia argentea, an ornamental plant from the amaranth family (Amaranthaceae). Besides the pain-causing activity, moroidin and celogentin C also exhibit potent inhibitory activity against tubulin polymerization and, therefore, have been considered as promising lead structures for developing new pain and cancer medications.

The development of moroidin-based drugs has been hindered by low isolation yields of moroidin peptides from source plants and challenging organic synthesis. Celogentin C has been successfully synthesized in 23 steps from simple amino acid building blocks, including a key C—H functionalization with a palladium-based catalyst to stereoselectively form the leucinetryptophan cross-link between two substrate molecules. Recently, this cross-linking methodology was further improved for stereoselective intramolecular macrocyclization of the left ring of celogentin C, shortening its total synthesis. However, scaled production and diversification of moroidins for drug development efforts remain difficult by a pure synthetic strategy. Therefore, the biosynthesis of moroidin in its source plants was studied to enable discovery of moroidin chemistry from other plants and heterologous production and diversification of these bicyclic peptides in alternative chassis organisms.

As used herein, the term “moroidin precursor peptide” refers to a peptide that includes an N-terminal leader domain, one or more core moroidin peptide domains, and, optionally, a C-terminal BURP domain or C-terminal DUF2775 domain. In some instances, one or more core moroidin peptide domains can be within a BURP domain. In some instances, one or more core moroidin peptide domains can be within a DUF2775 domain. In some instances, one or more core moroidin peptide domains are not within (e.g., outside) a BURP domain. In some instances, one or more core moroidin peptide domains can be within the N-terminal leader domain. In some instances, one or more core moroidin peptide domains are not within (e.g., outside) the N-terminal leader domain. In some embodiments, a moroidin precursor peptide includes from one to twenty core moroidin peptide domains. In some embodiments, a moroidin precursor peptide includes from one to ten core moroidin peptide domains. In some instances, moroidin precursor peptides can include more than twenty core moroidin peptide domains. In some embodiments, the moroidin precursor peptide includes a C-terminal BURP domain. In some embodiments, the moroidin precursor peptide, or biologically-active fragment thereof, can include a signal peptide sequence. For example, a signal peptide sequence can direct a moroidin precursor peptide, or biologically-active fragment thereof, through a portion of the secretory pathway and can facilitate localization to a particular organelle, such as a vacuole, which can be relevant for subsequent processing or conversion from a moroidin precursor peptide to a moroidin cyclic peptide. A signal peptide can be endogenous for a particular host cell or plant cell, or it can be heterologous. Typically, a signal peptide is located N-terminal to one or more core moroidin peptide domains. In some instances, a signal peptide can be part of an N-terminal leader domain. In certain host cells (e.g., mammalian or plant host cells), expression and/or secretion of a protein can be increased by using a signal sequence, such as a heterologous signal sequence. Therefore, in some embodiments, the moroidin precursor peptide includes a heterologous signal sequence at its N-terminus.

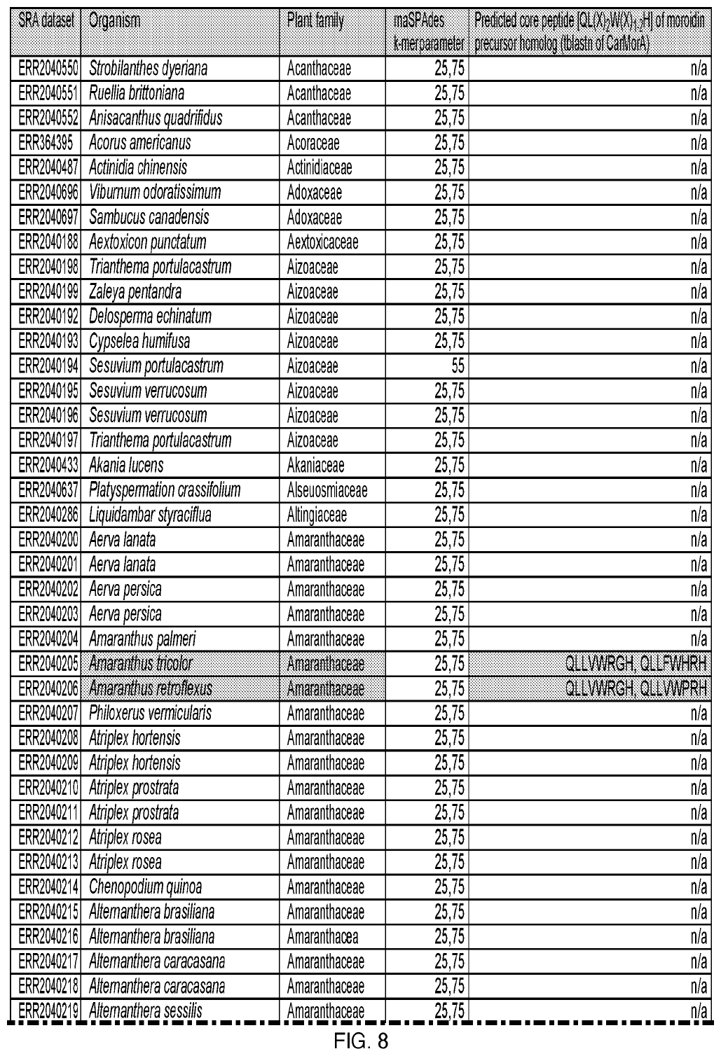

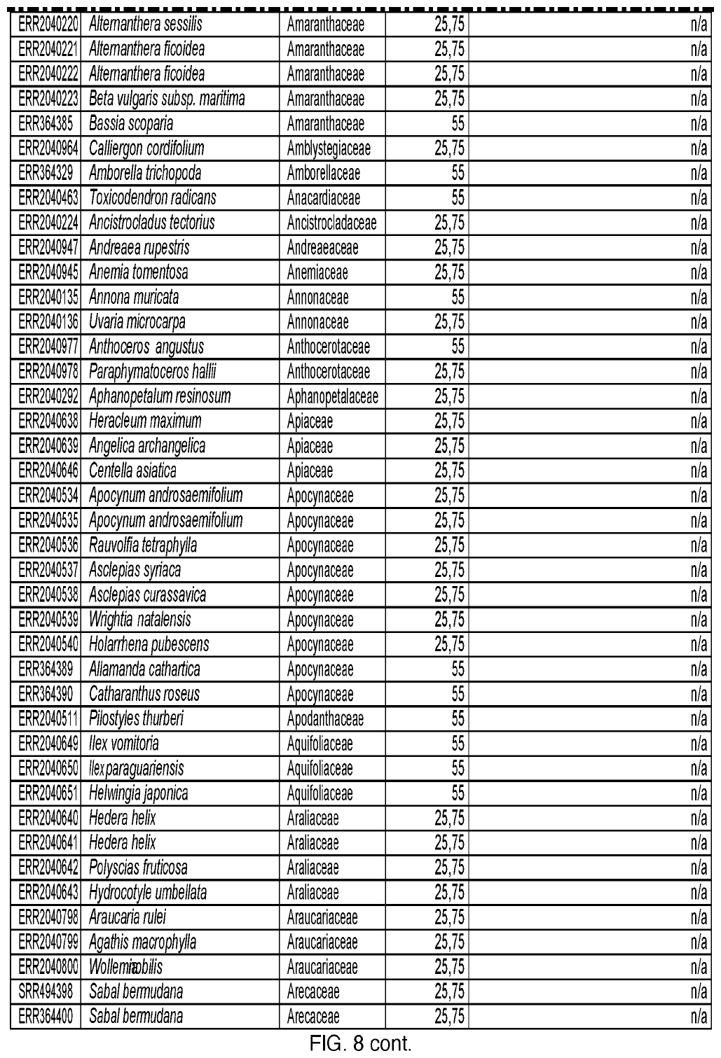

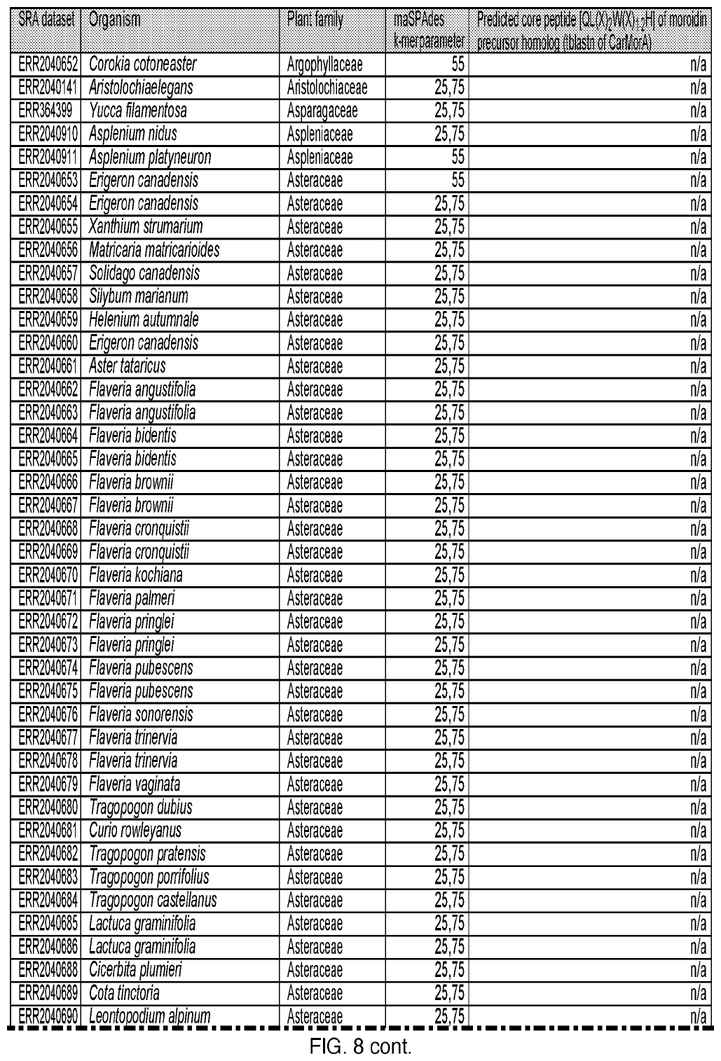

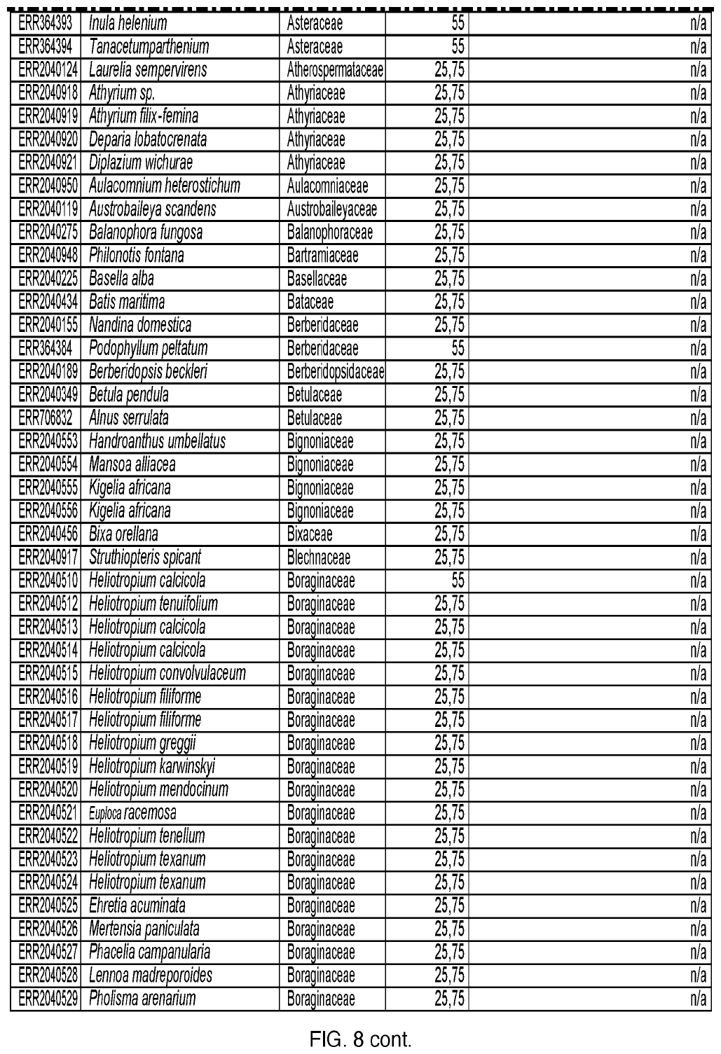

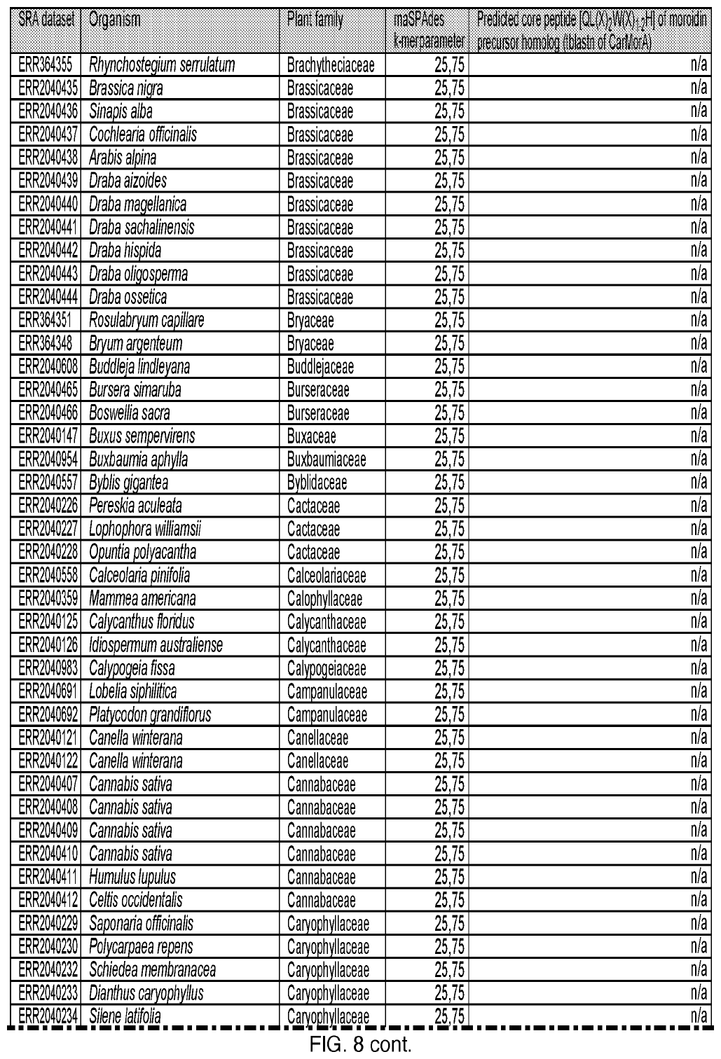

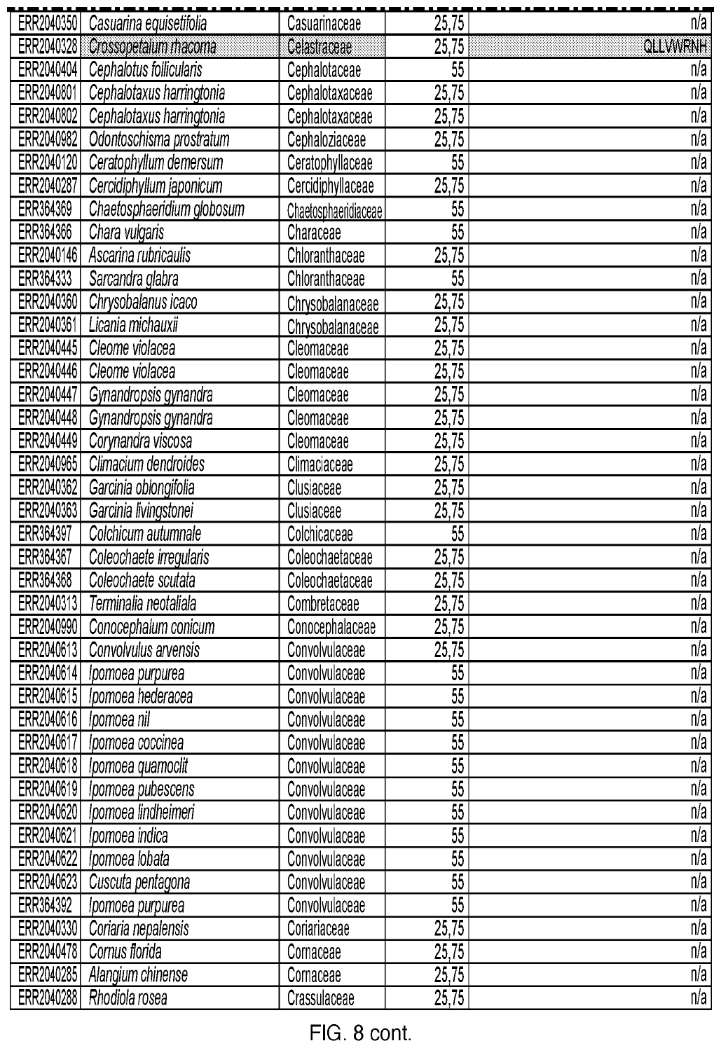

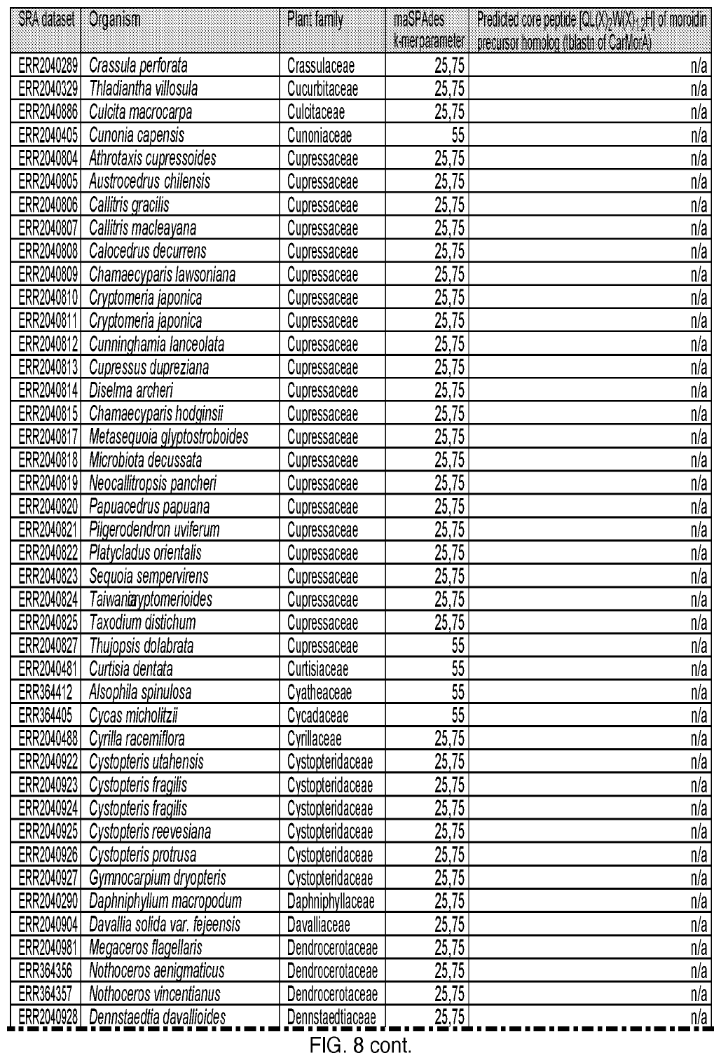

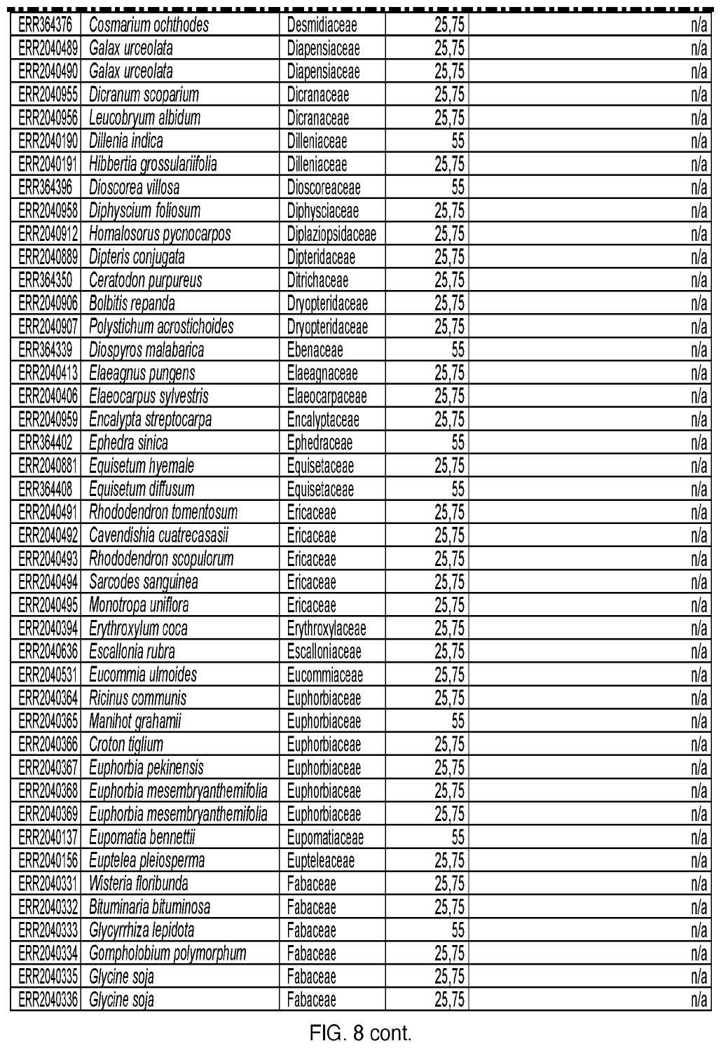

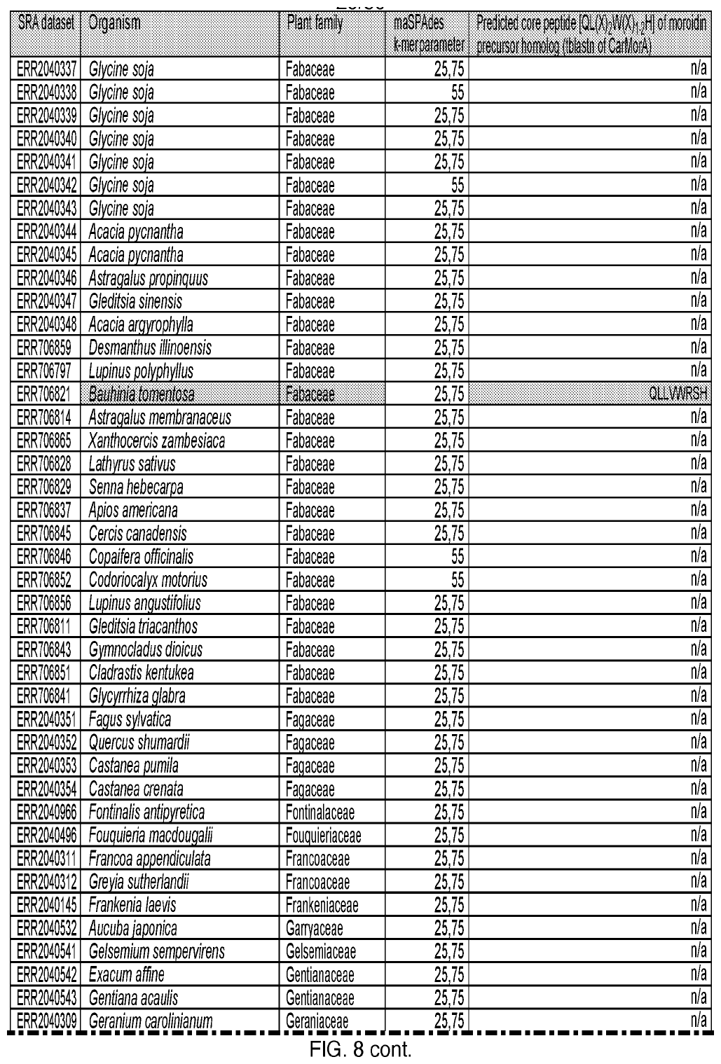

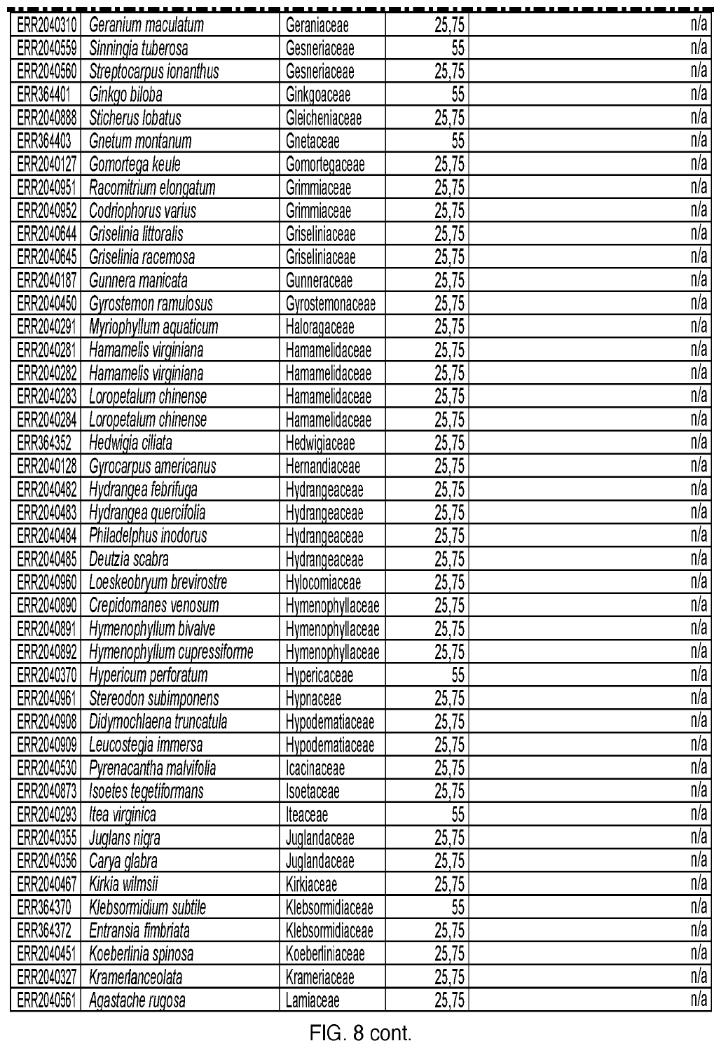

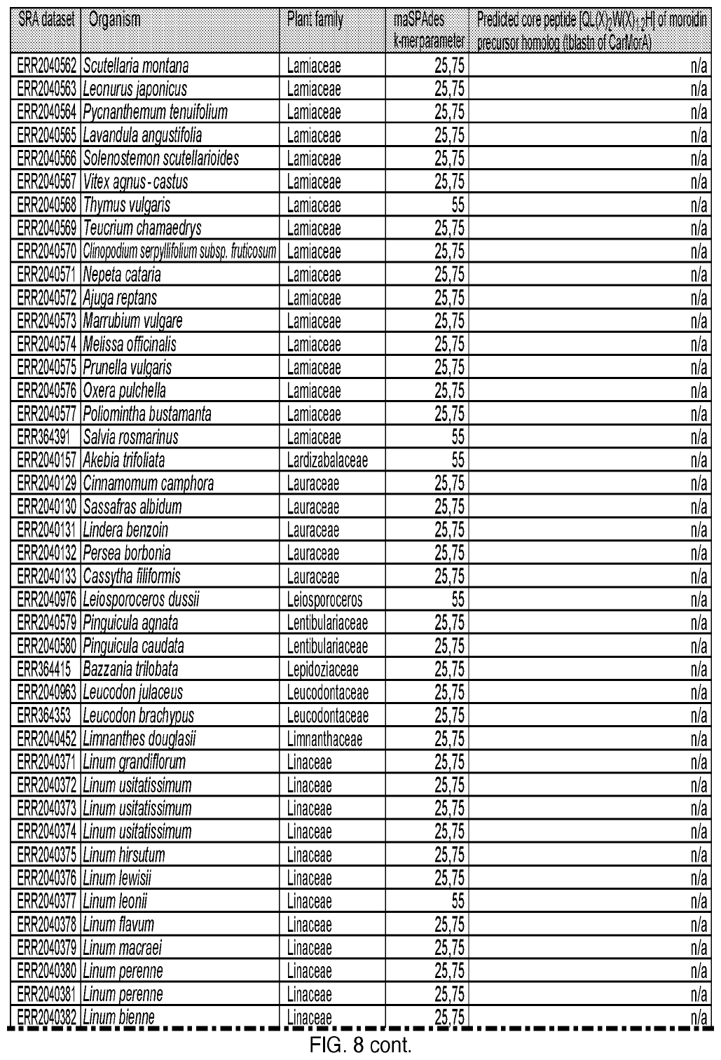

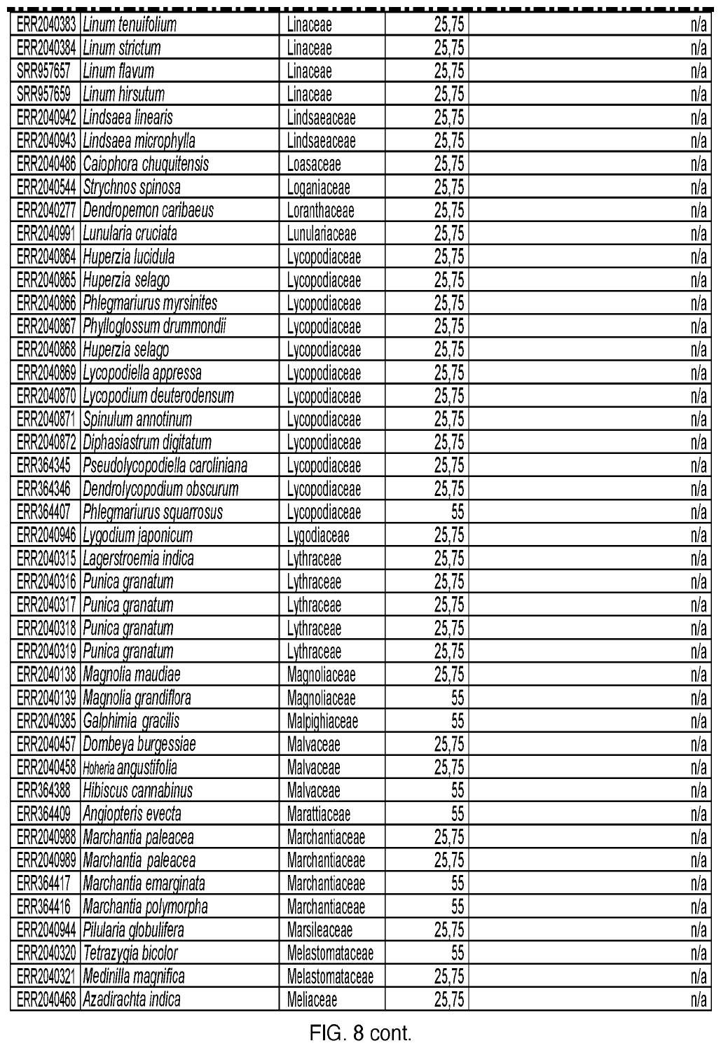

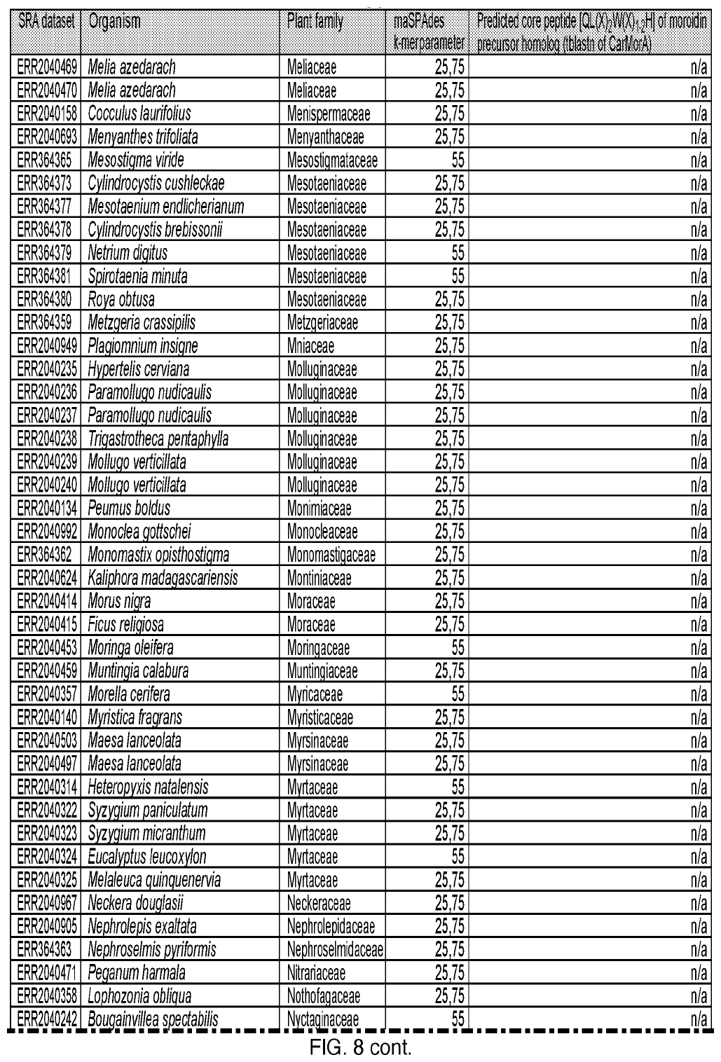

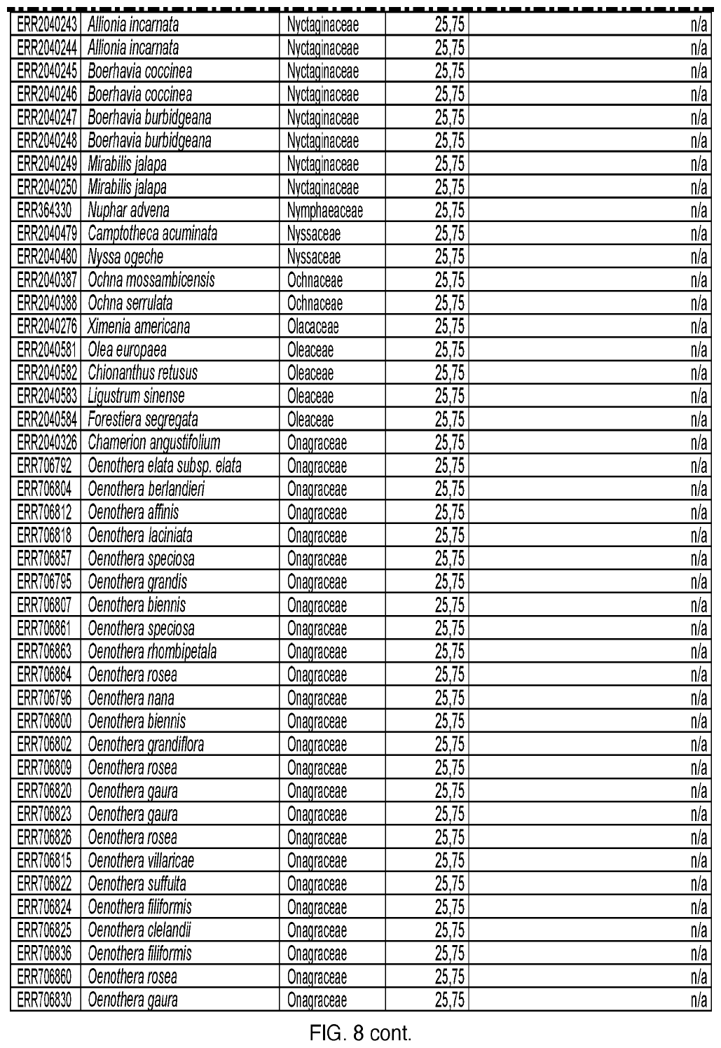

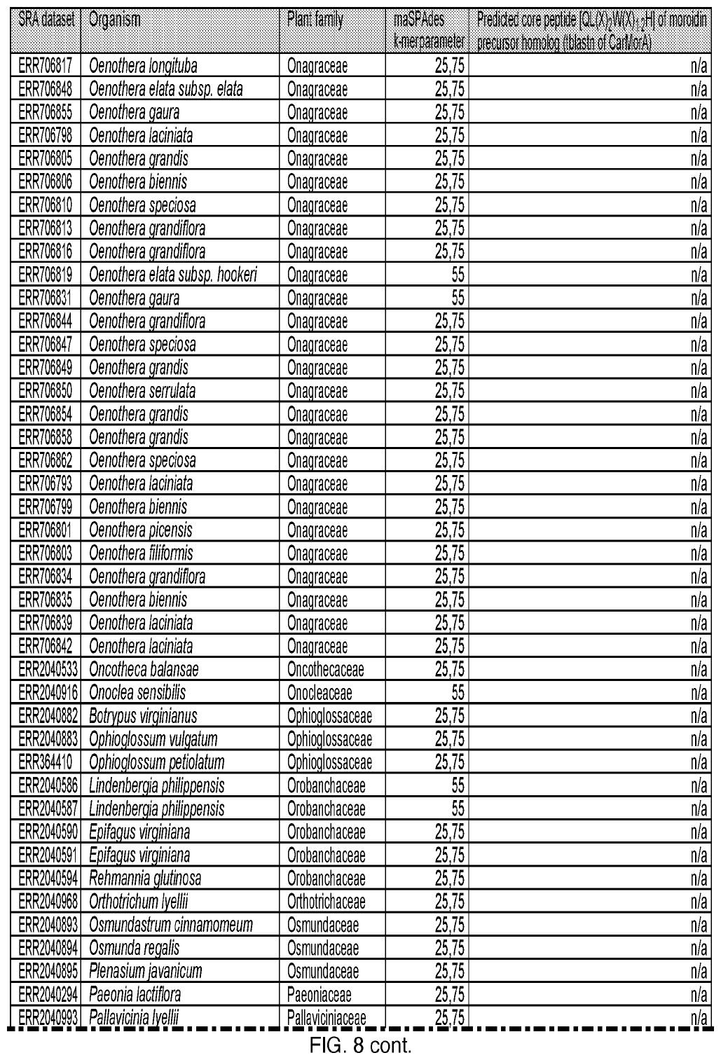

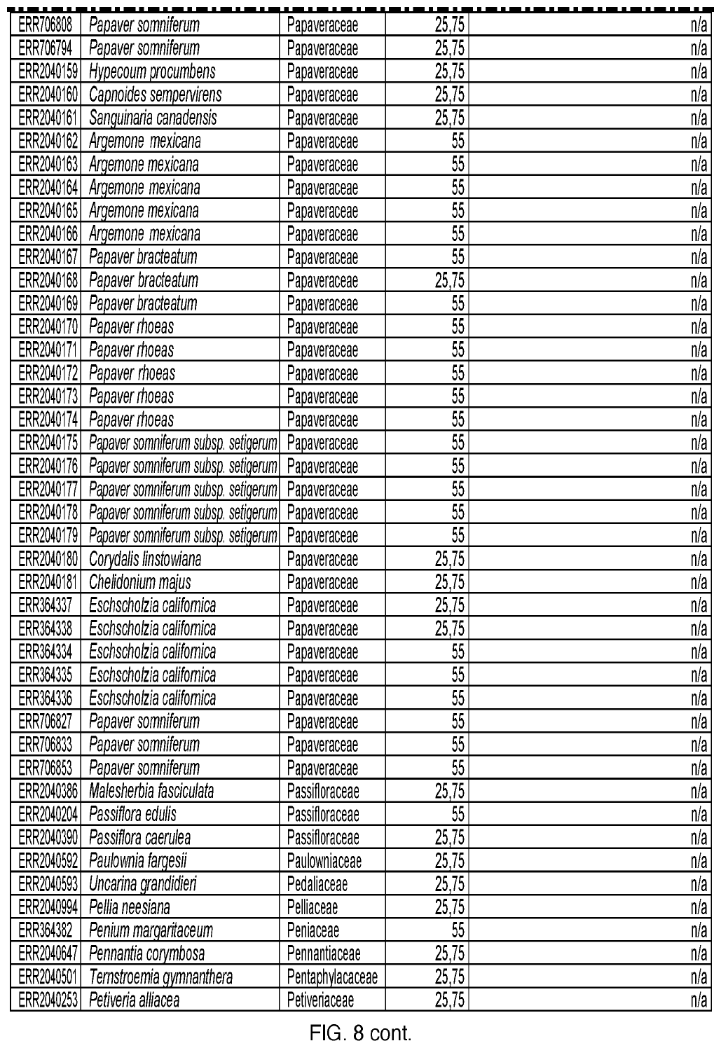

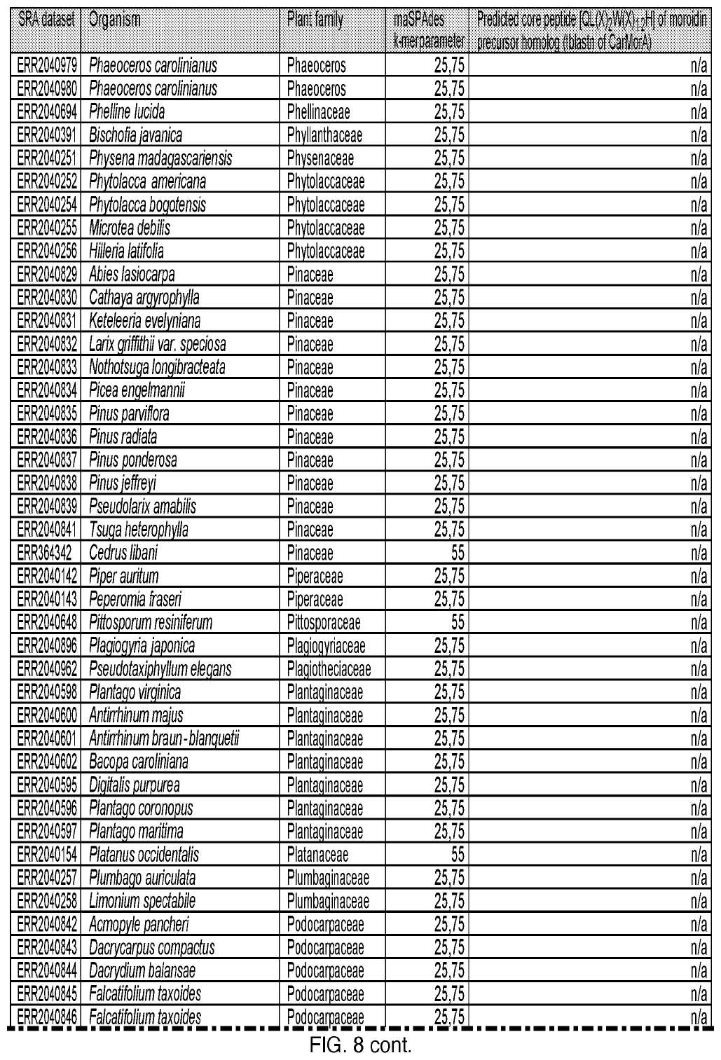

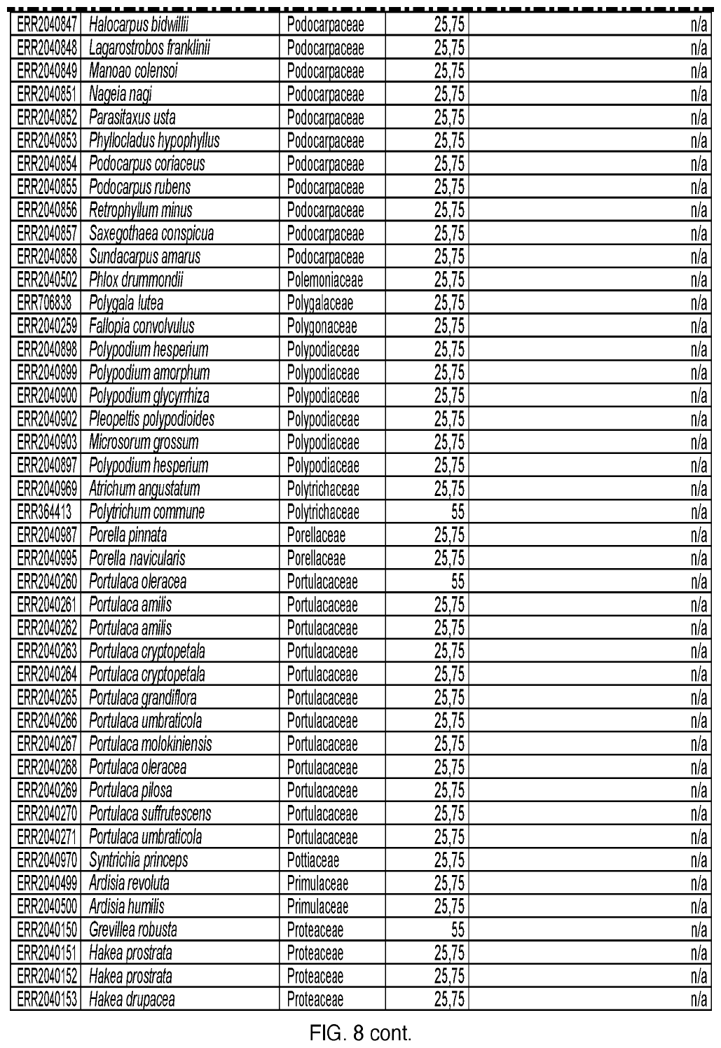

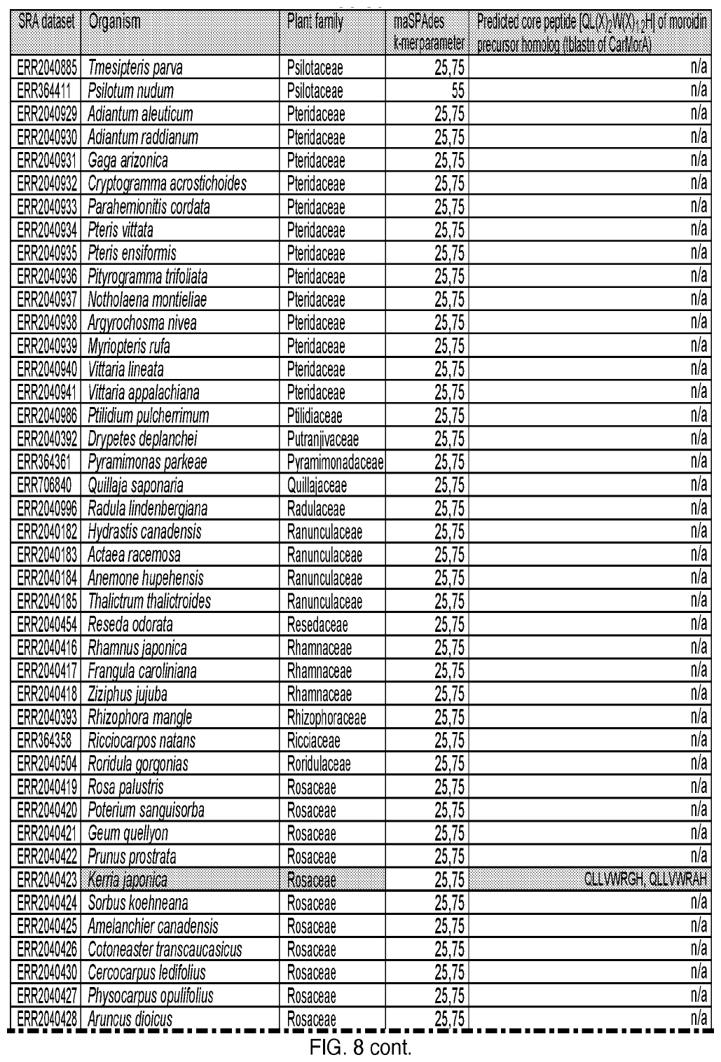

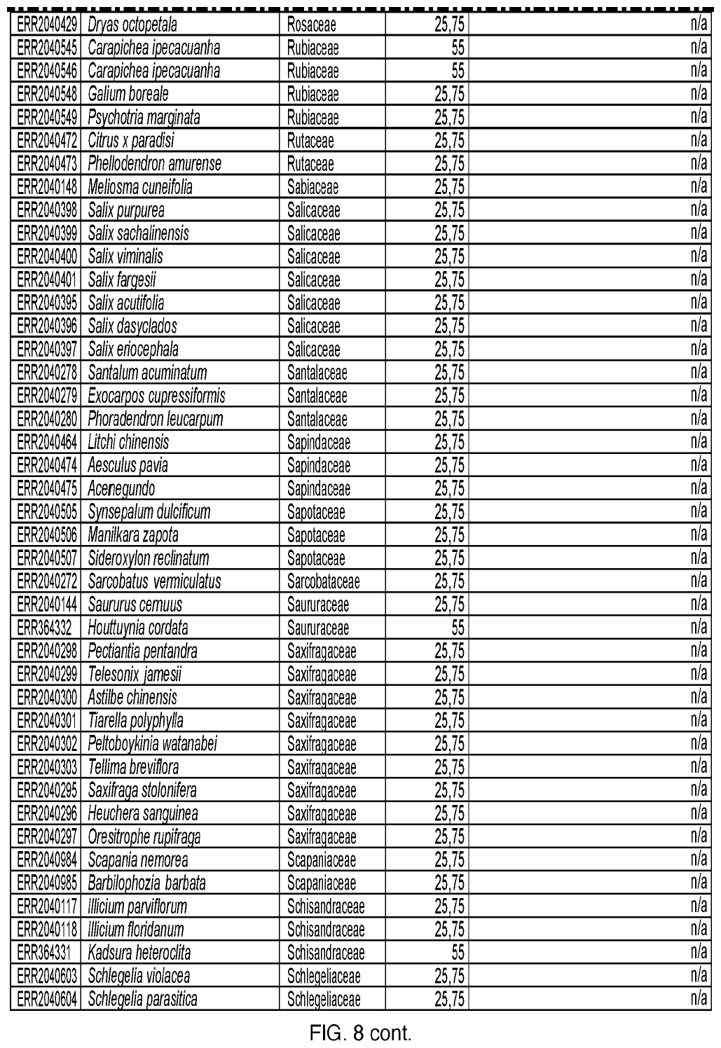

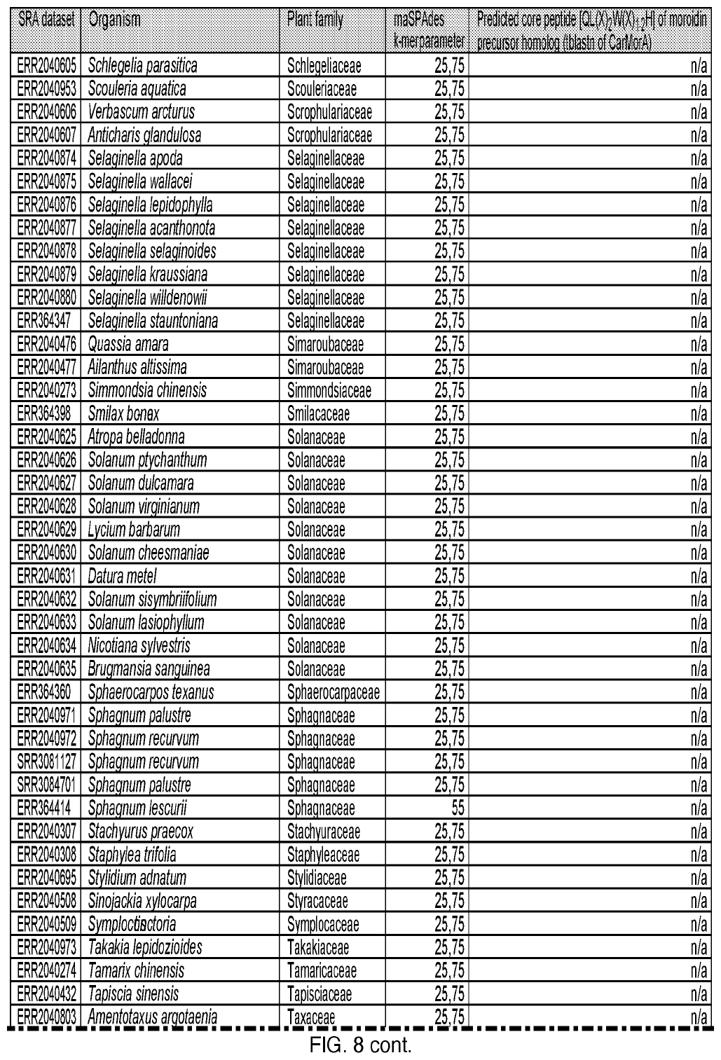

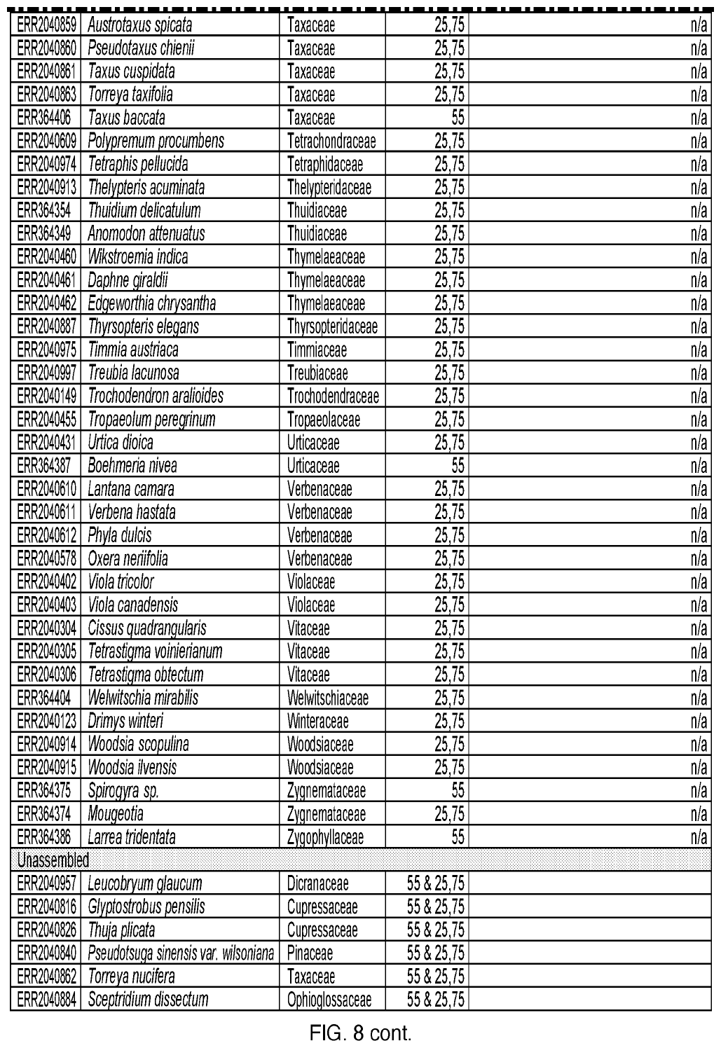

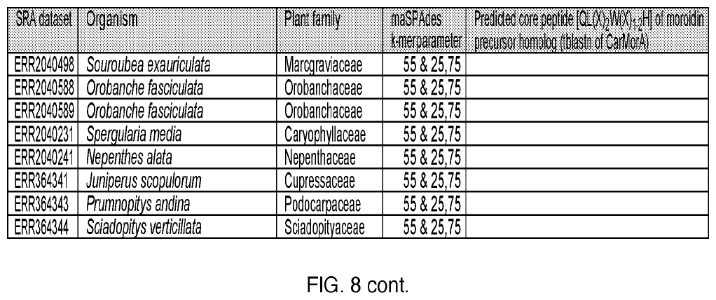

As used herein, the term “core moroidin peptide domain” refers to a peptide domain that includes seven or eight amino acids, frequently eight amino acids. The peptide is of the form QL(X)₂W(X)₁₋₂H (SEQ ID NO: 63), where X is any amino acid. For example, in some embodiments of interest, the peptide is of the form QLLVWRGH (SEQ ID NO: 59). For example, in some embodiments of interest, the peptide is of the form at least one core moroidin peptide domain comprises a variant of the sequence QL(X)₂W(X)₁₋₂H (SEQ ID NO: 63), wherein X is any amino acid and optionally wherein the W and/or the H is not mutated. In particular embodiments, X is any of the twenty-two naturally occurring amino acids. In particular embodiments, X is any of the twenty amino acids encoded by the universal genetic code. In some embodiments, a core moroidin peptide domain is a sequence listed in FIG. 8. In some embodiments, the core moroidin peptide domain differs in sequence from a naturally occurring core moroidin peptide domain. In some embodiments, the sequence of the moroidin precursor peptide, or biologically-active fragment thereof, differs from a naturally occurring sequence.

As used herein, the term “biologically-active fragment,” when referring to a moroidin precursor peptide, refers to a fragment of a moroidin precursor peptide that includes at least one core moroidin peptide domain and that can be converted to a moroidin cyclic peptide (e.g., in a host cell). Typically, the biologically-active fragment is cyclized in the host cell. In some instances, the biologically-active fragment may have shorter N-terminal or C-terminal domains compared to a moroidin precursor peptide. In some instances, biologically-active fragments can be fragments of naturally-occurring moroidin precursor peptides. In some instances, a biologically-active fragment can be a portion of a moroidin precursor peptide having at least one core moroidin peptide, which is embedded in, or linked to (e.g., at the N-terminus of, at the C-terminus of), a heterologous amino acid sequence that is not generally found in a moroidin precursor peptide.

In some embodiments, the invention provides a method of producing one or more moroidin cyclic peptides that includes: (a) providing a host cell that includes a transgene encoding a polypeptide that comprises one or more core moroidin peptide domains; (b) expressing the transgene in the host cell to thereby produce a polypeptide that includes one or more core moroidin peptide domains. In some embodiments, the polypeptide is converted to one or more moroidin cyclic peptides in the host cell.

As used herein, the term “moroidin cyclic peptide” refers to a bicyclic octapeptide, which is characterized by an N-terminal pyroglutamate and two side-chain macrocyclic linkages: (1) a C—C bond between the C6 of a tryptophan-indole at the fifth position and a 3-carbon of a leucine at the second position and (2) a C—N bond between the C2 of the same tryptophan-indole and the N1 of a C-terminal histidine-imidazole.

The BURP domain (Pfam 03181) is around 230 amino acid residues and has the following conserved features: two phenylalanine residues at its N-terminus; two cysteine residues; and four repeated cysteine-histidine motifs, arranged as: CH—X(10)-CH—X(25-27)-CH—X(25-26)-CH (SEQ ID NO: 64), where X can be any amino acid.

The DUF2775 domain (Pfam 10950) is a eukaryotic protein family which includes a number of plant organ-specific proteins. Their predicted amino acid sequence is often repetitive and suggests that these proteins could be exported and glycosylated. Multiple sequence alignment shows a highly conserved motif of 135 amino acids. This motif includes approximately 20 amino acids from the non-repeating area of the peptide, 2 tandem repeats and 1 truncated tandem repeat (Albornos et al., 2012). The first seven amino acids of the DUF2775 domain are typically KDXYXGW (SEQ ID NO: 65), where X can be any amino acid.

Embodiments described herein also include engineered nucleic acids that encode engineered moroidin precursor peptides (and engineered moroidin precursor peptides encoded by such engineered nucleic acids). An example is an engineered nucleic acid that encodes n number of core moroidin peptide domains, wherein n is an integer. The core moroidin peptide domains within an engineered moroidin precursor peptide can be identical or non-identical. Multiple identical core moroidin peptide domains can allow for increased production of a homogenous population of core moroidin peptides and moroidin cyclic peptides. Typically, n is an integer from 1 to 10, preferably from 5 to 10. In some instances, n can be greater than 10. In some instances, an engineered nucleic acid encodes from 5 to 10 identical moroidin precursor peptides. The core moroidin peptides domains are typically separated by an intervening sequence.

As used herein, “converting the moroidin precursor peptide, or biologically-active fragment thereof, to one or more moroidin cyclic peptides in a host cell,” “converted to one or more moroidin cyclic peptides in a host cell,” and similar phrases refer to one or more enzymatic reactions that convert a moroidin precursor peptide, or biologically-active fragment thereof, to one or more moroidin cyclic peptides. In some instances, conversion is facilitated by one or more enzymes that cyclizes the moroidin precursor peptide, or biologically-active fragment thereof. In some instances, conversion is catalyzed, in part, by one or more endopeptidases, such as an asparagine endopeptidase or an arginine endopeptidase, which acts N-terminal to a core moroidin peptide domain. In some instances, conversion is catalyzed by one or more glutamine cyclotransferases, which cyclize an N-terminal glutamine in a core moroidin peptide domain. In some instances, conversion is catalyzed by one or more exopeptidases. Conversion to a moroidin cyclic peptide can, but need not, occur within in a host cell.

Host cells include cells that are capable of converting a moroidin precursor peptide to a moroidin cyclic peptide, as well as cells that are incapable of converting a moroidin precursor peptide to a moroidin cyclic peptide. For example, a host cell can express a moroidin precursor peptide but lack one or more enzymes required to convert the moroidin precursor peptide to a moroidin cyclic peptide. In such circumstances, the moroidin precursor peptide can be isolated or obtained from the host cell and then converted to a moroidin cyclic peptide in another environment (e.g., in a cell free system, such as in a cell lysate (or fractionated cell lysate) from a source that is capable of converting a moroidin precursor peptide to a moroidin cyclic peptide).

In some embodiments, a moroidin precursor peptide can include a tag, which can be used to isolate the moroidin precursor peptide from a cell that expresses it. Such a tag can be useful for a manufacturing process that involves recombinant expression of a moroidin precursor peptide and subsequent cyclization using purified enzyme. In some embodiments, a nucleotide sequence encoding a moroidin precursor peptide is fused in-frame with a nucleotide sequence encoding an epitope tag, also known as an affinity tag, which can be useful for, e.g., protein purification. Examples of suitable epitope tags are known in the art and include FLAG, HA, His, GST, CBP, MBP, c-Myc, DHFR, GFP, CAT and others.

### Nucleic Acids

As used herein, the term “nucleic acid” refers to a polymer comprising multiple nucleotide monomers (e.g., ribonucleotide monomers or deoxyribonucleotide monomers). “Nucleic acid” includes, for example, DNA (e.g., genomic DNA and cDNA), RNA, and DNA-RNA hybrid molecules. Nucleic acid molecules can be naturally occurring, recombinant, or synthetic. In addition, nucleic acid molecules can be single-stranded, double-stranded or triple-stranded. In certain embodiments, nucleic acid molecules can be modified. In the case of a double-stranded polymer, “nucleic acid” can refer to either or both strands of the molecule.

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

In various embodiments, two nucleotide sequences, or two amino acid sequences, can have at least, e.g., 70%, 75%, 80%, 85%, 86%, 87%, 88%, 89%, 90%, 91%, 92%, 93%, 94%, 95%, 96%, 97%, 98%, 99%, or more, sequence identity. When ascertaining percent sequence identity to one or more sequences described herein, the sequences described herein are the reference sequences.

For many of the nucleotide sequences described herein, additional 5′- and 3′-nucleotides can be appended to the nucleotide sequence in order to perform Gibson cloning of the sequence into an expression vector. Gibson cloning utilizes Gibson assembly, an exonuclease-based method for joining DNA fragments.

### Vectors

The terms “vector”, “vector construct” and “expression vector” mean the vehicle by which a DNA or RNA sequence (e.g. a foreign gene) can be introduced into a host cell, so as to transform the host and promote expression (e.g. transcription and translation) of the introduced sequence. Vectors typically comprise the DNA of a transmissible agent, into which foreign DNA encoding a protein is inserted by, e.g., restriction enzyme technology. Some viral vectors comprise the RNA of a transmissible agent. A common type of vector is a “plasmid”, which generally is a self-contained molecule of double-stranded DNA that can readily accept additional (foreign) DNA and which can readily introduced into a suitable host cell. A large number of vectors, including plasmid and fungal vectors, have been described for replication and/or expression in a variety of eukaryotic and prokaryotic hosts.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

Gene delivery vectors generally include a transgene (e.g., nucleic acid encoding an enzyme) operably linked to a promoter and other nucleic acid elements required for expression of the transgene in the host cells into which the vector is introduced. Suitable promoters for gene expression and delivery constructs are known in the art. For bacterial host cells, suitable promoters, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xy1A and xy1B genes, and prokaryotic beta-lactamase gene (See e.g., Villa-Kamaroff et al., Proc. Natl. Acad. Sci. USA 75: 3727-3731, 1978), as well as the tac promoter (See e.g., DeBoer et al., Proc. Natl. Acad. Sci. USA 80: 21-25, 1983). Examples of promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Examples of yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488, 1992). For plant host cells, examples of suitable promoters include the cauliflower mosaic virus 35S promoter (CaMV 35S), and promoters (e.g., constitutive promoters) of genes that are highly expressed in plants (e.g., plant housekeeping genes, genes encoding Ubiquitin, Actin, Tubulin, or EIF (eukaryotic initiation factor)). Plant virus promoters can also be used. Additional useful plant promoters include those discussed in [50, 51], the entire contents of which are incorporated herein by reference. The selection of a suitable promoter is within the skill in the art. The recombinant plasmids can also comprise inducible, or regulatable, promoters for expression of a moroidin precursor peptide, or biologically-active fragment thereof, in cells.

Various gene delivery vehicles are known in the art and include both viral and non-viral (e.g., naked DNA, plasmid) vectors. Viral vectors suitable for gene delivery are known to those skilled in the art. Such viral vectors include, e.g., vector derived from the herpes virus, baculovirus vector, lentiviral vector, retroviral vector, adenoviral vector and adeno-associated viral vector (AAV). Vectors derived from plant viruses can also be used, such as the viral backbones of the RNA viruses Tobacco mosaic virus (TMV), Potato virus X (PVX) and Cowpea mosaic virus (CPMV), and the DNA geminivirus Bean yellow dwarf virus. The viral vector can be replicating or non-replicating.

Non-viral vectors include naked DNA and plasmids, among others. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and such vectors may be introduced into many appropriate host cells, using methods disclosed or cited herein or otherwise known to those skilled in the relevant art.

In certain embodiments, the vector comprises a transgene operably linked to a promoter. The transgene encodes a biologically-active molecule, such as a moroidin precursor peptide described herein.

To facilitate the introduction of the gene delivery vector into host cells, the vector can be combined with different chemical means such as colloidal dispersion systems (macromolecular complex, nanocapsules, microspheres, beads) or lipid-based systems (oil-in-water emulsions, micelles, liposomes).

Some embodiments relate to a vector comprising a nucleic acid encoding moroidin precuror peptide, or biologically-active fragment thereof, described herein. In certain embodiments, the vector is a plasmid, and includes any one or more plasmid sequences such as, e.g., a promoter sequence, a selection marker sequence, or a locus-targeting sequence. Suitable plasmid vectors include p423TEF 2μ, p425TEF 2μ, and p426TEF 2μ. Another suitable vector is pHis8-4 (Whitehead Institute, Cambridge, Massachusetts, United States of America). Another suitable vector is pEAQ-HT.

Although the genetic code is degenerate in that most amino acids are represented by multiple codons (called “synonyms” or “synonymous” codons), it is understood in the art that codon usage by particular organisms is nonrandom and biased towards particular codon triplets. Accordingly, in some embodiments, the vector includes a nucleotide sequence that has been optimized for expression in a particular type of host cell (e.g., through codon optimization). Codon optimization refers to a process in which a polynucleotide encoding a protein of interest is modified to replace particular codons in that polynucleotide with codons that encode the same amino acid(s), but are more commonly used/recognized in the host cell in which the nucleic acid is being expressed. In some aspects, the polynucleotides described herein are codon optimized for expression in a bacterial cell, e.g., E. coli. In some aspects, the polynucleotides described herein are codon optimized for expression in a yeast cell, e.g., S. cerevisiae. In some aspects, the polynucleotides described herein are codon optimized for expression in a tobacco cell, e.g., N. benthamiana.

### Host Cells

A wide variety of host cells can be used in the present invention, including fungal cells, bacterial cells, plant cells, insect cells, and mammalian cells.

In some embodiments, the host cell is a fungal cell, such as a yeast cell and an Aspergillus spp cell. A wide variety of yeast cells are suitable, such as cells of the genus Pichia, including Pichia pastoris and Pichia stipitis; cells of the genus Saccharomyces, including Saccharomyces cerevisiae; cells of the genus Schizosaccharomyces, including Schizosaccharomyces pombe; and cells of the genus Candida, including Candida albicans.

In some embodiments, the host cell is a bacterial cell. A wide variety of bacterial cells are suitable, such as cells of the genus Escherichia, including Escherichia coli; cells of the genus Bacillus, including Bacillus subtilis; cells of the genus Pseudomonas, including Pseudomonas aeruginosa; and cells of the genus Streptomyces, including Streptomyces griseus.

In some embodiments, the host cell is a plant cell. A wide variety of cells from a plant are suitable, including cells from a Nicotiana benthamiana plant. In some embodiments, the plant belongs to a genus selected from the group consisting of Arabidopsis, Beta, Glycine, Helianthus, Solanum, Triticum, Oryza, Brassica, Medicago, Prunus, Malus, Hordeum, Musa, Phaseolus, Citrus, Piper, Sorghum, Daucus, Manihot, Capsicum, and Zea. In some embodiments, the host cell is a plant cell from the Amaranthaceae family. In some embodiments, the plant cell is an Amaranthus genus plant cell, such as an Amaranthus hypochondriacus plant cell. In some embodiments, the plant cell is a Beta genus plant cell, such as a Beta vulgaris plant cell. In some embodiments, the plant cell is a Chenopodium genus plant cell, such as a Chenopodium quinoa plant cell. In some embodiments, the plant cell is a Fabaceae family plant cell. In some embodiments, the plant cell is a Glycine genus plant cell, such as a Glycine max plant cell. In some embodiments, the plant cell is a Medicago genus plant cell, such as a Medicago truncatula plant cell. In some embodiments, the plant cell is a Solanaceae family plant cell. In some embodiments, the plant cell is a Solanum genus plant cell, such as a Solanum melongena plant cell or a Solanum tuberosum plant cell. In some embodiments, the plant cell is a Nicotiana genus plant cell, such as a Nicotiana benthamiana plant cell. In some embodiments, the plant cell is a Capsicum genus plant cell, such as a Capsicum annuum plant cell.

In some embodiments, the host cell is an insect cell, such as a Spodoptera frugiperda cell, such as Spodoptera frugiperda Sf9 cell line and Spodoptera frugiperda Sf21

In some embodiments, the host cell is a mammalian cell.

In some embodiments, the host cell is an Escherichia coli cell. In some embodiments, the host cell is a Nicotiana benthamiana cell. In some embodiments, the cell is a Saccharomyces cerevisiae cell.

As used herein, the term “host cell” encompasses cells in cell culture and also cells within an organism (e.g., a plant). In some embodiments, the host cell is part of a transgenic plant.

Some embodiments relate to a host cell comprising a vector as described herein. In certain embodiments, the host cell is an Escherichia coli cell, a Nicotiana benthamiana cell, or a Saccharomyces cerevisiae cell.

In some embodiments, the host cells are cultured in a cell culture medium, such as a standard cell culture medium known in the art to be suitable for the particular host cell.

### Methods of Making Transgenic Host Cells

Described herein are methods of making a transgenic host cell. The transgenic host cells can be made, for example, by introducing one or more of the vector embodiments described herein into the host cell.

In some embodiments, the method comprises introducing into a host cell a vector that includes a nucleic acid transgene that encodes a moroidin precursor peptide, or a biologically-active fragment thereof. The moroidin precursor peptide, or biologically-active fragment thereof, can include one or more core moroidin peptide domains.

In some embodiments, one or more of the nucleic acids are integrated into the genome of the host cell. In some embodiments, the nucleic acids to be integrated into a host genome can be introduced into the host cell using any of a variety of suitable methodologies known in the art, including, for example, CRISPR-based systems (e.g., CRISPR/Cas9; CRISPR/Cpf1), TALEN systems and Agrobacterium-mediated transformation. However, as those skilled in the art would recognize, transient transformation techniques can be used that do not require integration into the genome of the host cell. In some embodiments, nucleic acid (e.g., plasmids) can be introduced that are maintained as episomes, which need not be integrated into the host cell genome.

In certain embodiments, the nucleic acid is introduced into a tissue, cell, or seed of a plant cell. Various methods of introducing nucleic acid into the tissue, cell, or seed of plants are known to one of ordinary skill in the art, such as protoplast transformation. The particular method can be selected based on several considerations, such as, e.g., the type of plant used. For example, a floral dip method is a suitable method for introducing genetic material into a plant. In other embodiments, agroinfiltration can be useful for transient expression in plants. In certain embodiments, the nucleic acid can be delivered into the plant by an Agrobacterium.

In some embodiments, a host cell is selected or engineered to have increased activity of the synthesis pathway.

Some of the methods described herein include assaying for an activity of interest. For example, crude extract from a host cell that expresses a moroidin precursor peptide and/or moroidin cyclic peptide, or a moroidin cyclic peptide isolated from the host cell, can be assayed for an activity of interest. An example of an activity of interest is modulation (enhancement or inhibition) of fungal or bacterial growth, such as the ability to inhibit growth of a pathogenic fungal or bacterial species or the ability to promote growth of a potentially desirable fungal or bacterial species. Another example of an activity of interest is a protease inhibitor activity, which can include inhibition of a viral, bacterial, fungal, or mammalian protease.

### Exemplification

### Results

### Characterization of Candidate DUF2775 Precursor Peptides of Moroidins in Plants

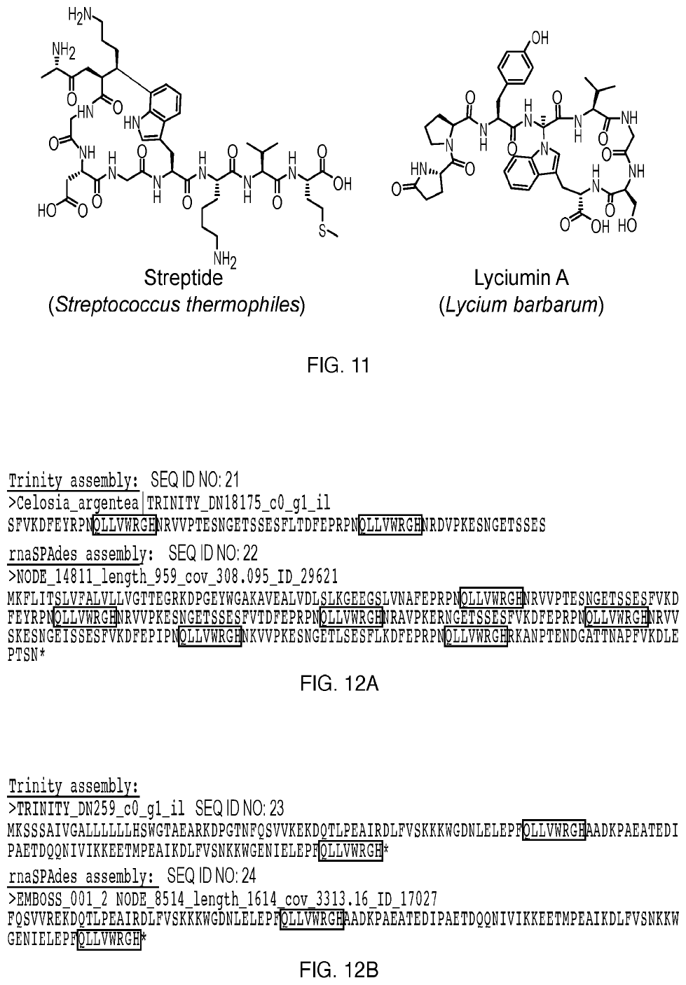

It has been hypothesized that moroidin is a nonribosomal peptide due to its unusual macrocyclization chemistry. However, available plant genomes do not contain genes encoding large nonribosomal peptide synthetases and, recently, peptide natural products with tryptophan macrocyclization functionalities similar to moroidin were characterized as ribosomal peptides from bacteria and plants. Streptide, a cyclic peptide from Streptococcal bacteria contains a C—C crosslink between the C7 of a tryptophan-indole and the β-carbon of a lysine13, and the lyciumins are plant RiPPs with C—N bonds between the α-carbon of a glycine and the nitrogen of a tryptophan-indole (FIG. 11). It is hypothesized that moroidins may also be RiPPs.

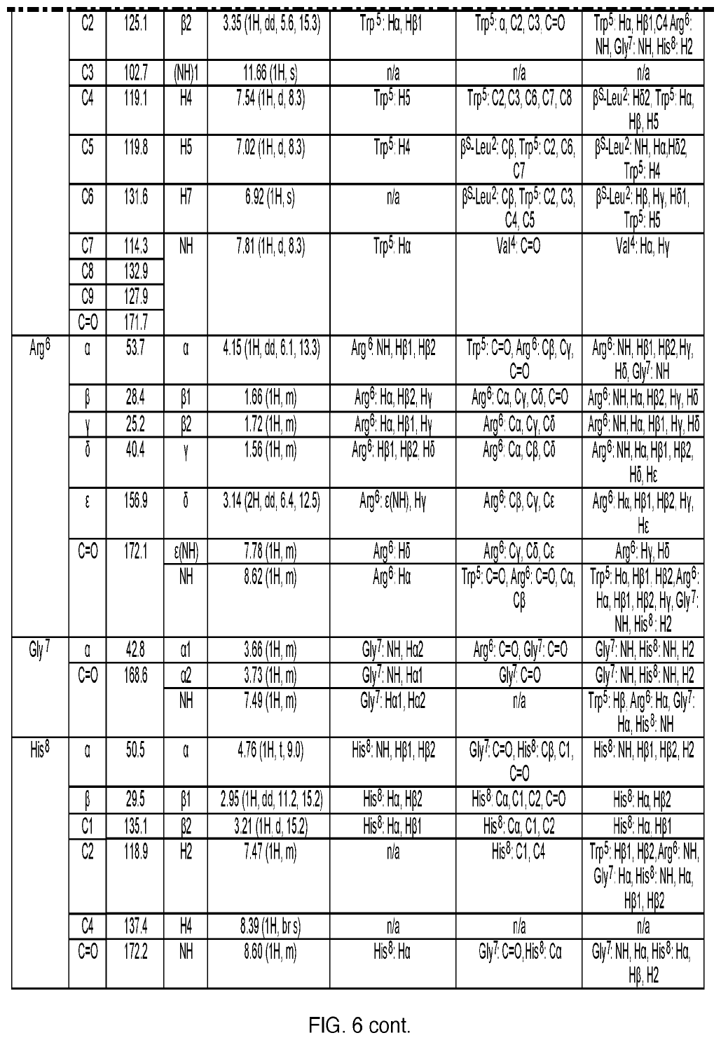

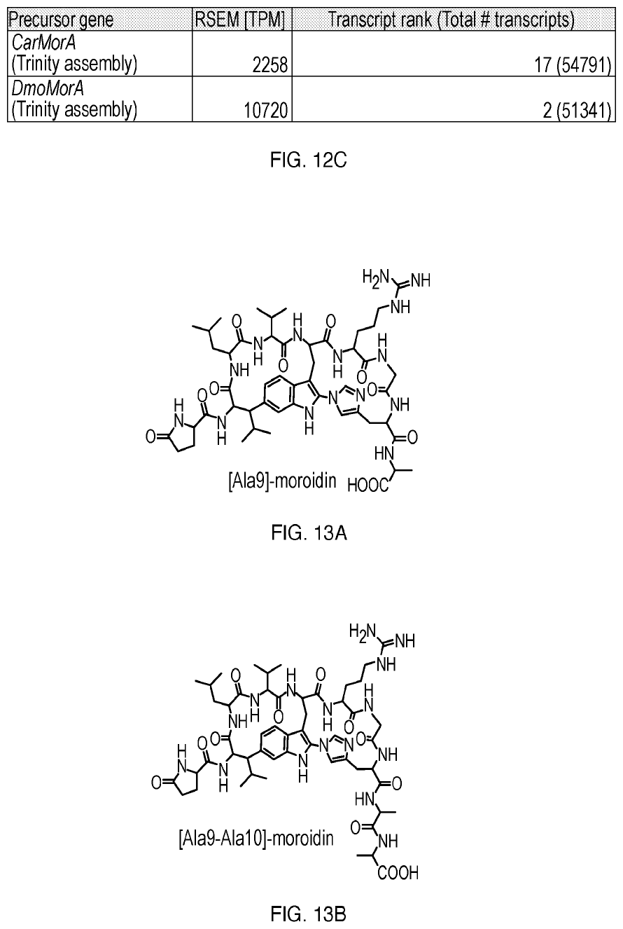

To test this hypothesis, corresponding moroidin precursor genes in source plants were identified. C. argentea var. cristata and D. moroides plants were obtained, and it was first confirmed that moroidin is produced in the flowers and seeds of C. argentea and the leaves of D. moroides using liquid-chromatography-mass-spectrometry (LC-MS) and nuclear magnetic resonance (NMR) (FIG. 1B, FIG. 6). De novo transcriptomes of the C. argentea flower tissue and the D. moroides leaf tissue were generated and queried for the putative moroidin core peptide sequence QLLVWRGH (SEQ ID NO: 59) by a tblastn search. A transcript encoding multiple copies of the predicted moroidin core peptide was identified from the de novo flower transcriptome of C. argentea and the corresponding full-length coding sequence (CDS), CarMorA, was successfully cloned from C. argentea flower cDNA. CarMorA belongs to the DUF2775 protein family (Pfam10950) of unknown function, and contains six repeats of the potential moroidin core peptide (FIG. 1C). Querying the leaf transcriptome of D. moroides identified a transcript encoding two copies of the predicted moroidin core peptide. Cloning of the corresponding CDS from D. moroides leaf cDNA yielded DmoMorA, which also encodes a precursor peptide of the DUF2775 family with two repeats (FIG. 1C). The correct assembly of CarMorA from RNA-seq data was achieved by the de novo transcriptome assembler rnaSPAdes, which when executed with a long kmer assembly parameter, outperformed Trinity in the assembly of these tandem repetitive DUF2775 peptides (FIG. 12A-C).

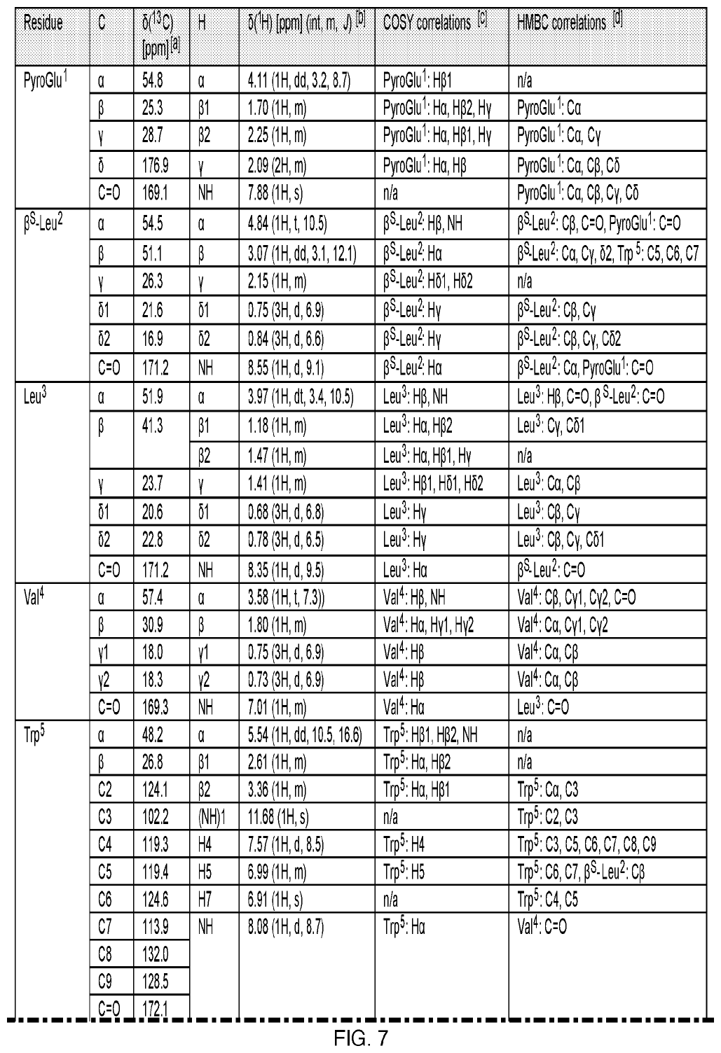

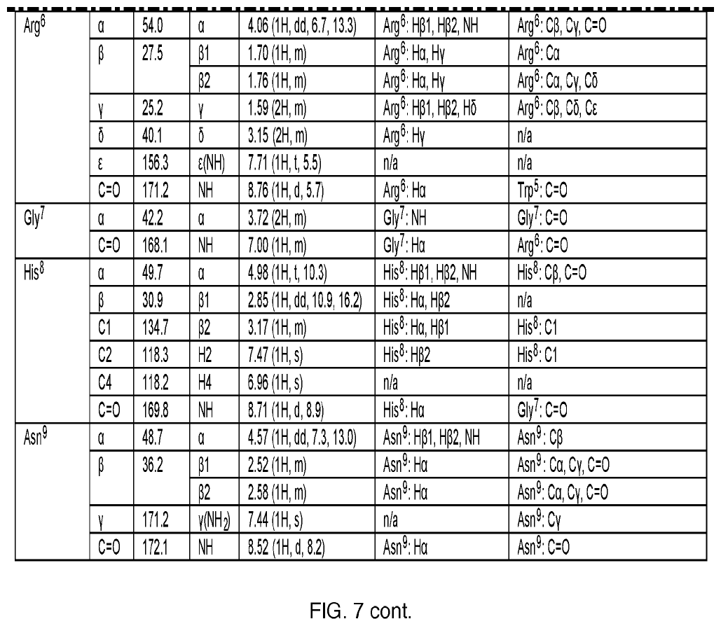

Both candidate moroidin precursor genes were highly expressed in their source tissues: CarMorA is the 17th highest expressed gene in the C. argentea flower transcriptome and DmoMorA is the 2nd highest expressed gene in D. moroides leaf transcriptome, respectively (FIG. 12A-C). Consistent with the CarMorA and DmoMorA protein sequences, from the plant peptide extracts several C-terminally extended moroidins that match each precursor sequence downstream of the moroidin core peptides were characterized. In particular, a moroidin derivative with a C-terminal asparagine extension was isolated and structurally elucidated from C. argentea flower extracts (FIG. 7), whereas two moroidin-derivatives with one or two C-terminal alanine extensions were detected in D. moroides leaf extract (FIG. 13A-B). The identification of highly expressed CarMorA and DmoMorA with repeats encoding moroidin core peptides and detection of C-terminally extended moroidin derivatives as predicted by the corresponding sequences in CarMorA and DmoMorA strongly indicate that moroidins are RiPPs and CarMorA and DmoMorA are precursor peptides for moroidin biosynthesis in C. argentea and D. moroides, respectively.

### Gene-Guided Discovery of Moroidin Peptides in Genome- and Transcriptome-Sequenced Plants

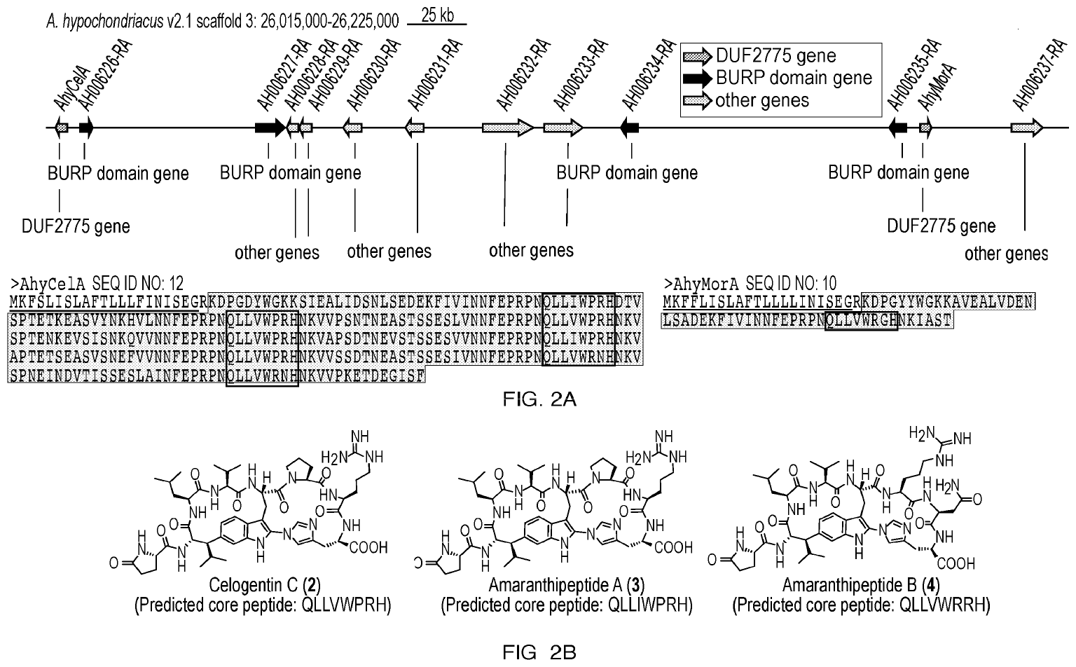

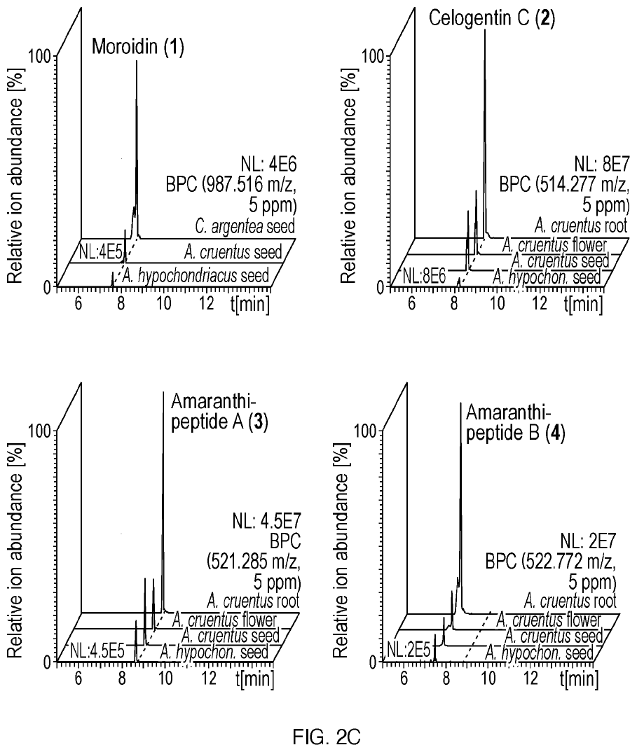

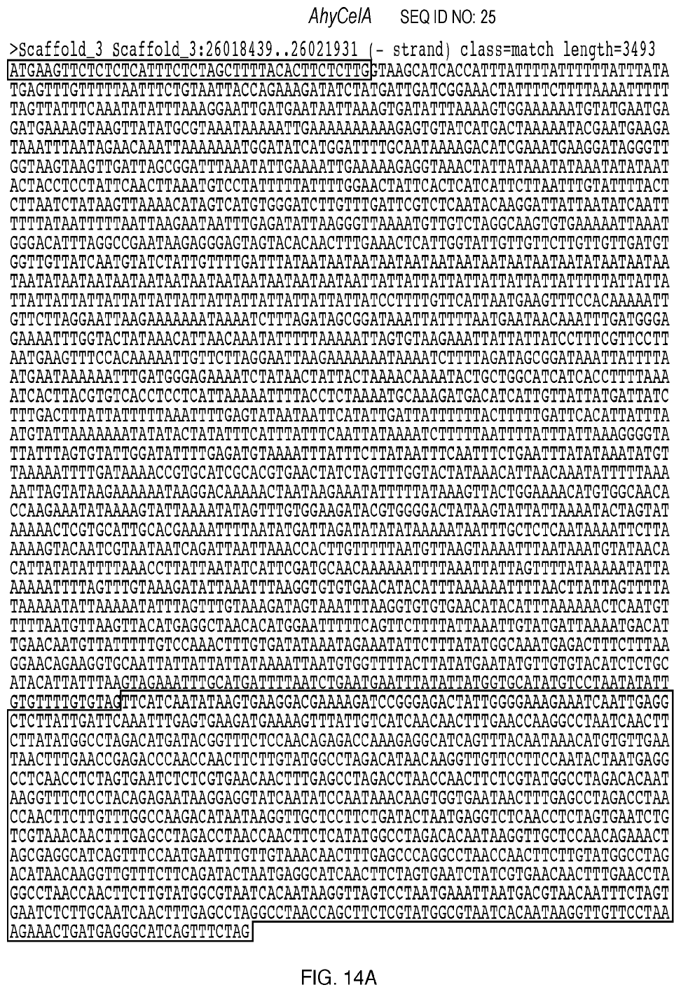

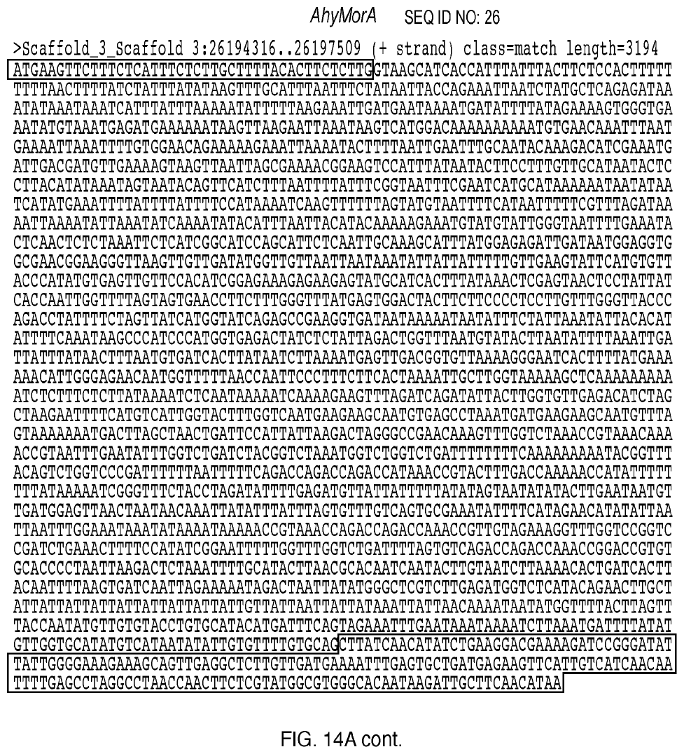

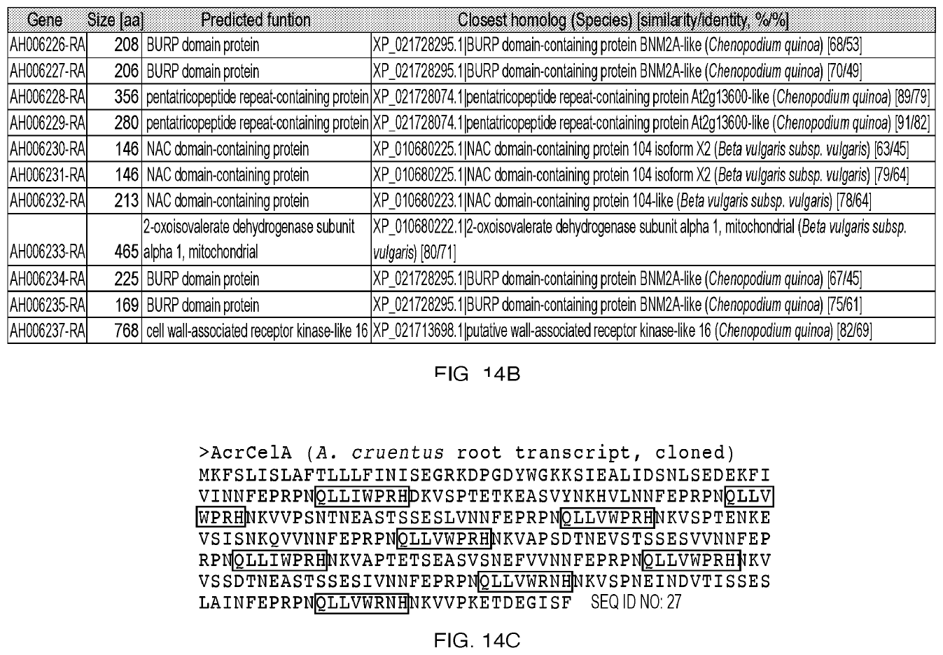

With sequences of putative moroidin precursors in hand, additional moroidin chemistry and producers were identified by searching plant genomes and transcriptomes for homologs of the moroidin precursor genes. For moroidin peptide genome mining, 91 plant genomes available through the Joint Genome Institute Phytozome (v12.1) were queried by tblastn for homologs of CarMorA. Two closely related CarMorA homologs were identified in the genome of the dietary grain amaranth (Amaranthus hypochondriacus), which, like C. argentea, belongs to the Amaranthaceae family. The two predicted moroidin precursor genes from A. hypochondriacus, which were not present in the original genome annotation, also encode DUF2775 family proteins, and are co-localized in the same genomic locus (FIG. 2A) with both predicted genes having a two-intron-one-exon structure (FIG. 14A-C). One of the predicted amaranth moroidin precursors, AhyCelA, contains six repeats with three different core peptide sequences, including a core peptide for celogentin C (QLLVWPRH) (SEQ ID NO: 60. In contrast, the other precursor, AhyMorA, contains one moroidin core peptide (QLLVWRGH) (SEQ ID NO: 59) (FIG. 2A). Both precursor sequences were further confirmed by cloning from cDNA or de novo transcriptome assembly (FIG. 14A-C). Based on these newly identified moroidin peptide genotypes, the presence of the corresponding bicyclic peptides by LC-MS metabolic profiling of extracts were prepared from various tissues of A. hypochondriacus and the closely related species A. cruentus. Moroidin was detected in seed extract of A. hypochondriacus, whereas celogentin C was detected in A. hypochondriacus seed extract as well as A. cruentus root, flower and seed extracts. In addition, two new moroidin analogs were detected, namely amaranthipeptides A and B, in A. hypochondriacus seed extract as well as A. cruentus root, flower and seed extracts, which match the two other core peptides (QLLIWPRH (SEQ ID NO: 61) and QLLVWRNH (SEQ ID NO: 66), respectively) present in AhyCelA and its A. cruentus homolog AcrCelA (FIGS. 2B and 2C). Interestingly, AhyCelA and AhyMorA are also in vicinity of several other genes encoding BURP domain proteins (Pfam 03181) in the amaranth genome (FIG. 2A and FIG. 14A-C), which were recently characterized as lyciumin precursor peptides.

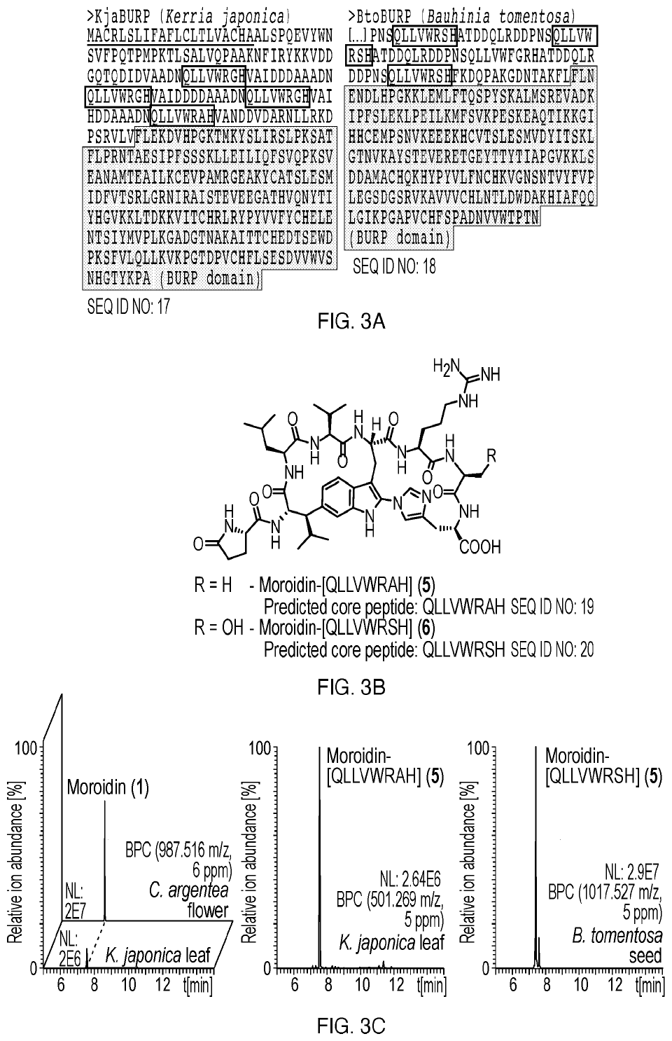

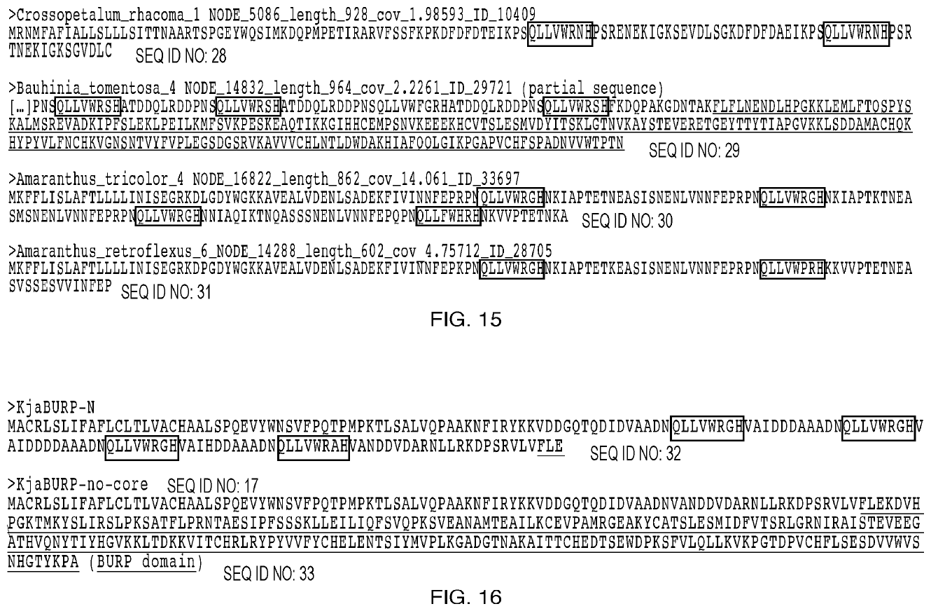

For moroidin peptide transcriptome mining, the RNA-seq datasets made available through the 1 kp project were used. Given the results of improved DUF2775 precursor gene assembly using rnaSPAdes (FIG. 12A-C), de novo reassembly of transcriptomes of 793 plant species using rnaSPAdes starting from raw sequencing reads deposited by the 1 kp project, representing a total of 317 land plant families were used (FIG. 8). Subsequently, a search for moroidin genotypes in these reassembled transcriptomes by tblastn using CarMorA as a query was conducted. This exercise readily identified several candidate moroidin precursor genes distributed across diverse plant families that extend beyond Amaranthaceae and Urticaceae. These newly identified moroidin-genotype-containing plant families include Celastraceae, Fabaceae and Rosaceae. In specific, candidate moroidin precursors from maidenberry (Crossopetalum rhacoma), Japanese kerria (Kerria japonica) and yellow bauhinia (Bauhinia tomentosa) (FIG. 3A and FIG. 15) were found, which enabled the subsequent LC-MS detection of predicted moroidin and moroidin-[QLLVWRAH] (SEQ ID NO: 41) chemotypes in the leaf extract of K. japonica and predicted moroidin-[QLLVWRSH] chemotype in the seed extract of B. tomentosa (FIGS. 3B and 3C and FIG. 15). The characterization of moroidin chemistry from additional plant families highlights that new plant peptide chemistry can be discovered by searching moroidin precursor genes in plant genomes and transcriptomes. Surprisingly, both of the identified moroidin precursors from K. japonica and Bauhinia sp. contain a C-terminal BURP domain. As BURP domains have recently been characterized in precursor peptides of lyciumins, another class of cyclic plant ribosomal peptides with a tryptophan macrocyclization, were investigated for their role in moroidin biosynthesis.

### Ribosomal Biosynthesis of Moroidin by a BURP Domain Precursor Peptide

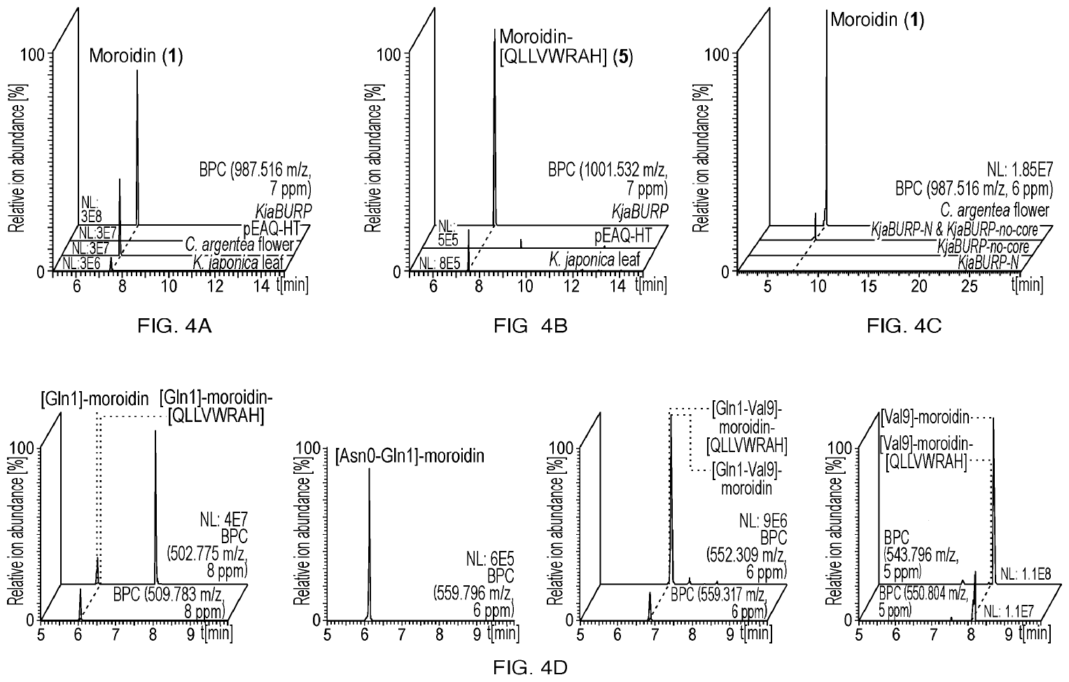

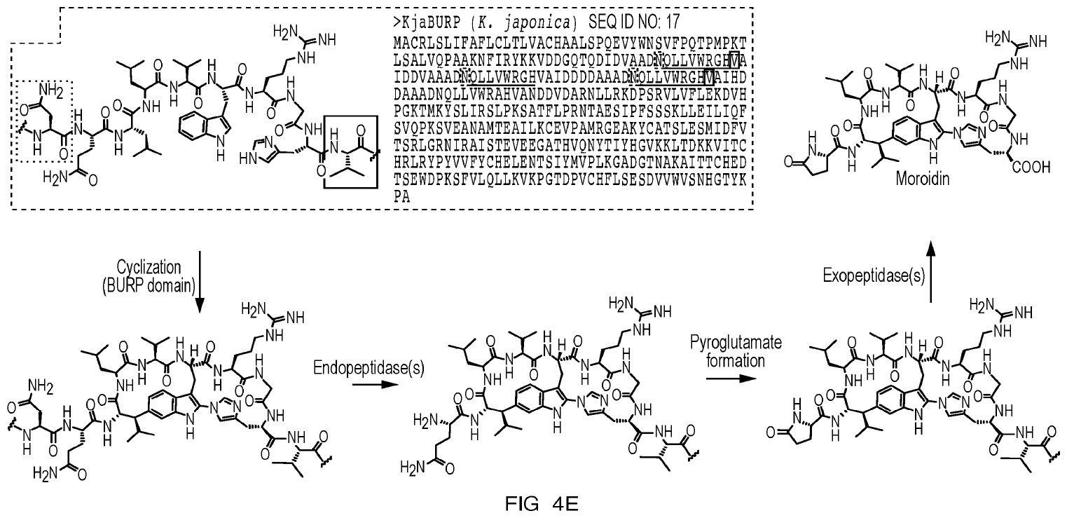

Next whether the BURP domains have a catalytic role in plant peptide biosynthesis was investigated. To test this, cloned the predicted moroidin precursor gene from K. japonica, KjaBURP was cloned and expressed it heterologously in Nicotiana benthamiana via Agrobacterium-mediated transient expression in order to verify its role as a moroidin precursor. LC-MS analysis of the peptide extract of N. benthamiana leaves six days after Agrobacterium infiltration of KjaBURP showed mass signals for moroidin and a moroidin analog matching the core peptide QLLVWRAH (SEQ ID NO: 19) (FIGS. 4A and 4B and FIG. 16), which confirmed the ribosomal origin of moroidins. Subsequently, two gene constructs of KjaBURP were constructed: (1) KjaBURP-N which contains just the N terminus with the moroidin core peptides and (2) KjaBURP-no-core which contains the full-length BURP domain sequence without the moroidin core peptides (FIG. 16). Transient expression of each of these synthetic gene constructs in N. benthamiana did not result in moroidin biosynthesis (FIG. 4C). However, when KjaBURP-N and KjaBURP-no-core were co-expressed in N. benthamiana, moroidin biosynthesis could be reconstituted (FIG. 4C and FIG. 16), suggesting that the precursor peptide BURP domain catalyzes the bicyclization of moroidin core peptides in tobacco. In addition, transgenic expression of candidate DUF2775-domain precursors AcrCelA, DmoMorA or CarMorA, which have no C-terminal BURP domain, did not yield moroidin in N. benthamiana.

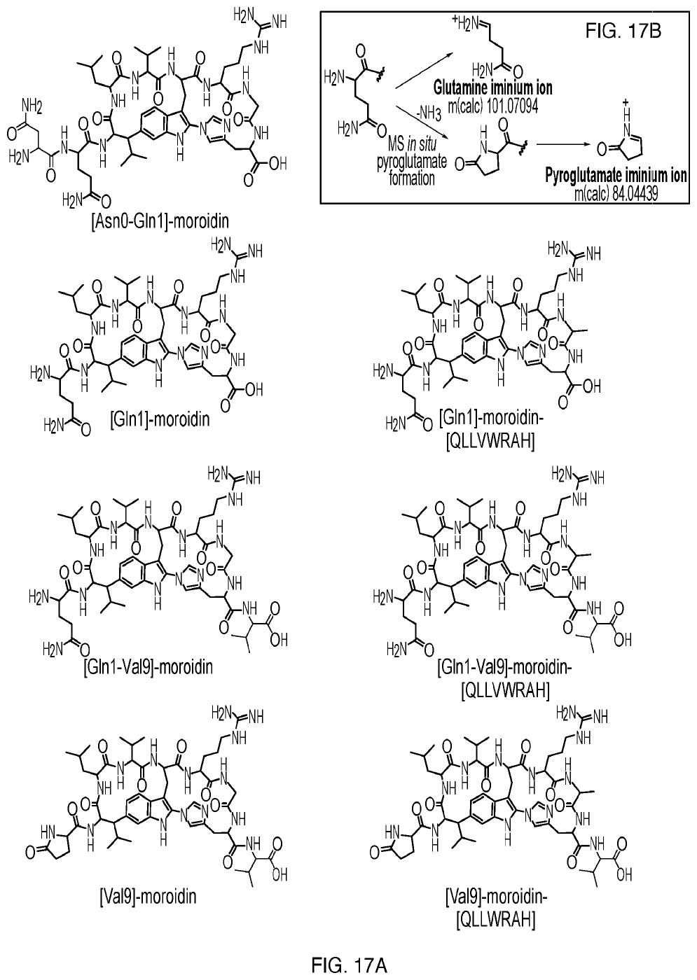

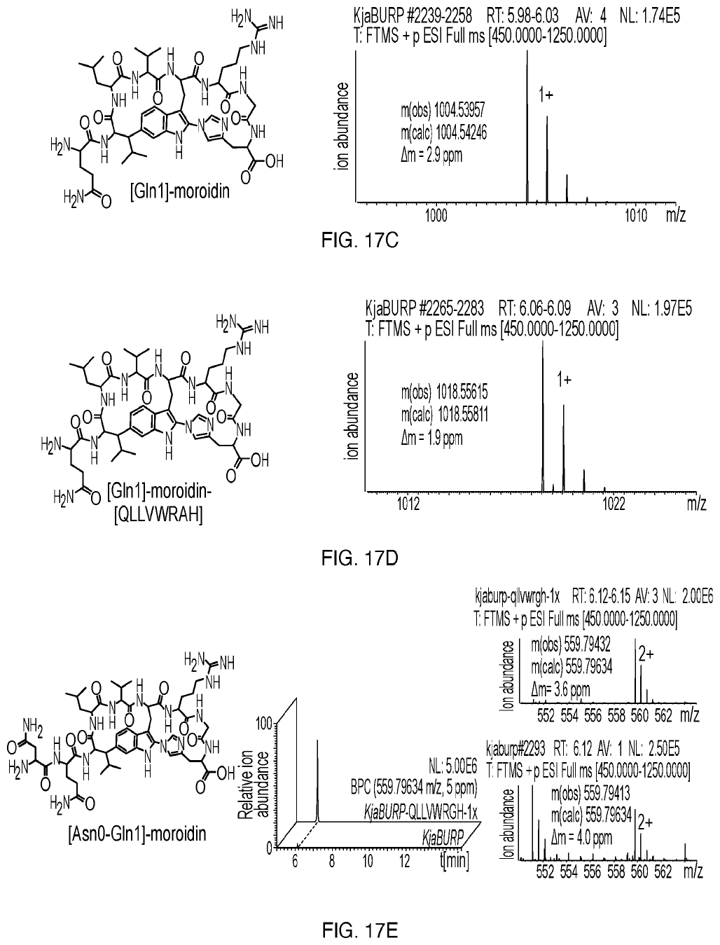

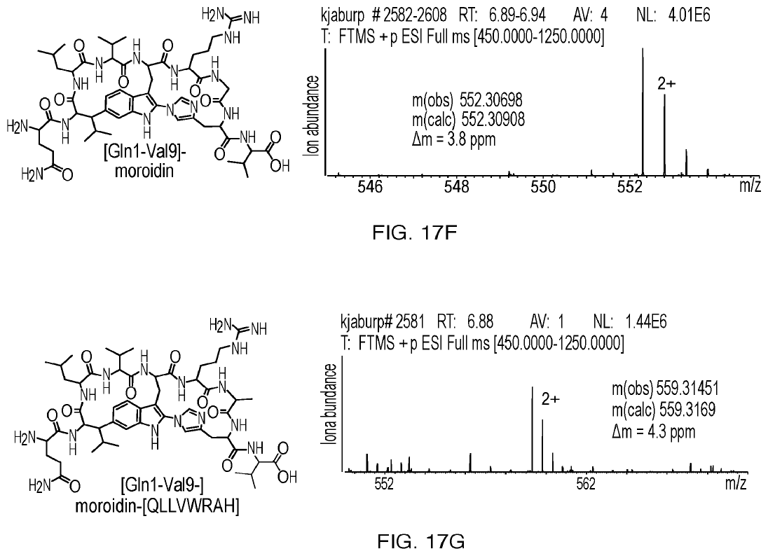

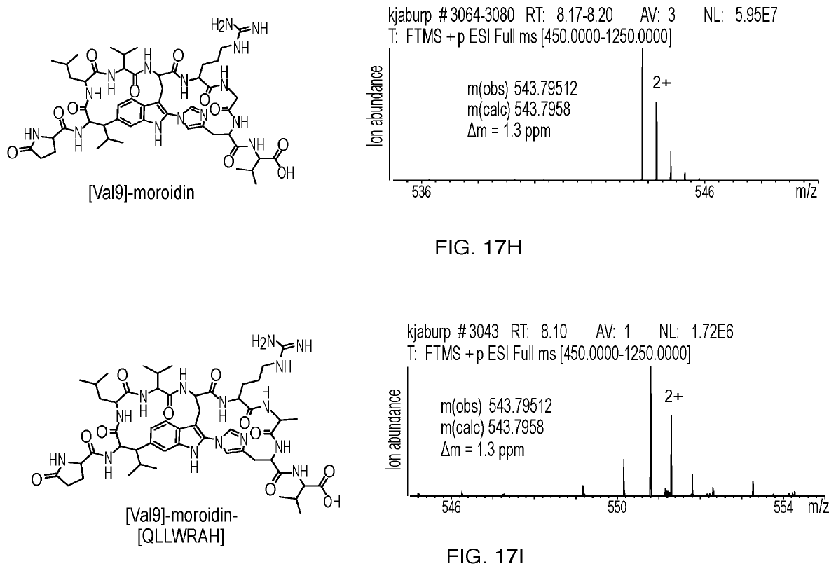

Based on transient gene expression studies of KjaBURP in N. benthamiana, a biosynthetic proposal for moroidin peptides in Kerria japonica could be formulated (FIG. 4E). First, KjaBURP is translated by the ribosome to yield the precursor peptide KjaBURP with an N-terminal domain with four repeats including three core peptides for moroidin and one core peptide for moroidin-[QLLVWRAH] (SEQ ID NO: 41). Next, the BURP domain catalyzes the bicyclization of the core peptides in the N-terminal domain as a substrate. This is supported by the fact that no linear moroidin core peptides were detected from extracts of N. benthamiana transiently expressing KjaBURP or extracts of K. japonica. Whether the BURP domain catalyzes the bicyclization of core peptides in cis or in trans remains to be determined; however, the KjaBURP-N and KjaBURP-no-core co-expression result indicates that it can act in trans. Subsequently, the modified N-terminus is likely proteolytically cleaved by endopeptidases to yield a moroidin derivative with an N-terminal glutamine. A moroidin derivative with an N-terminal asparagine extension was detected from extracts of N. benthamiana transiently expressing KjaBURP, indicating non-specific N-terminal proteolysis (FIG. 4D and FIG. 17 A-I). In addition, several derivatives of moroidin and moroidin-[QLLVWRAH] (SEQ ID NO: 41) with N-terminal glutamines were detected, indicating a biosynthetic moroidin intermediate with an uncyclized N-terminus (FIG. 4D and FIG. 17A-I). The N-terminal glutamine is most likely cyclized by a glutamine cyclotransferase, which was shown to be involved in pyroglutamate formation in lyciumin biosynthesis. Finally, the C-terminus of moroidin is matured by exopeptidase cleavage, which is supported by the detection of several moroidin and moroidin-[QLLVWRAH] (SEQ ID NO: 41) derivatives with C-terminal valine extensions (FIG. 4D and FIG. 17A-I). Ultimately, the in vivo reconstitution of moroidin biosynthesis by co-expression of the precursor core peptide domain and the precursor BURP domain and the detection of peptides with C- and N-terminally extended moroidin motifs suggest the BURP domain as a plant peptide cyclase that catalyzes moroidin bicyclization prior to N- and C-terminal protection and maturation.

### Moroidin Diversification in Transgenic Tobacco

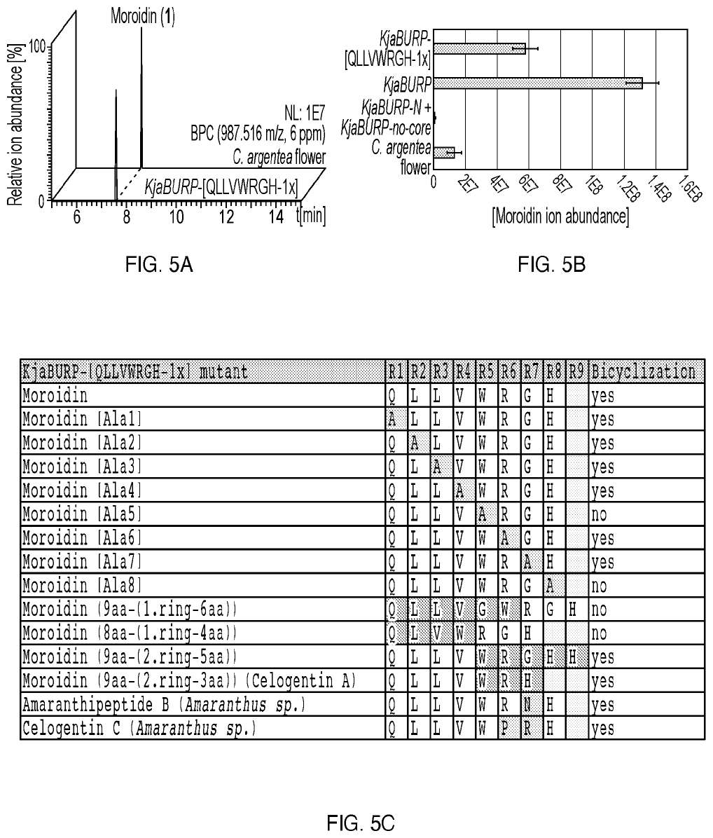

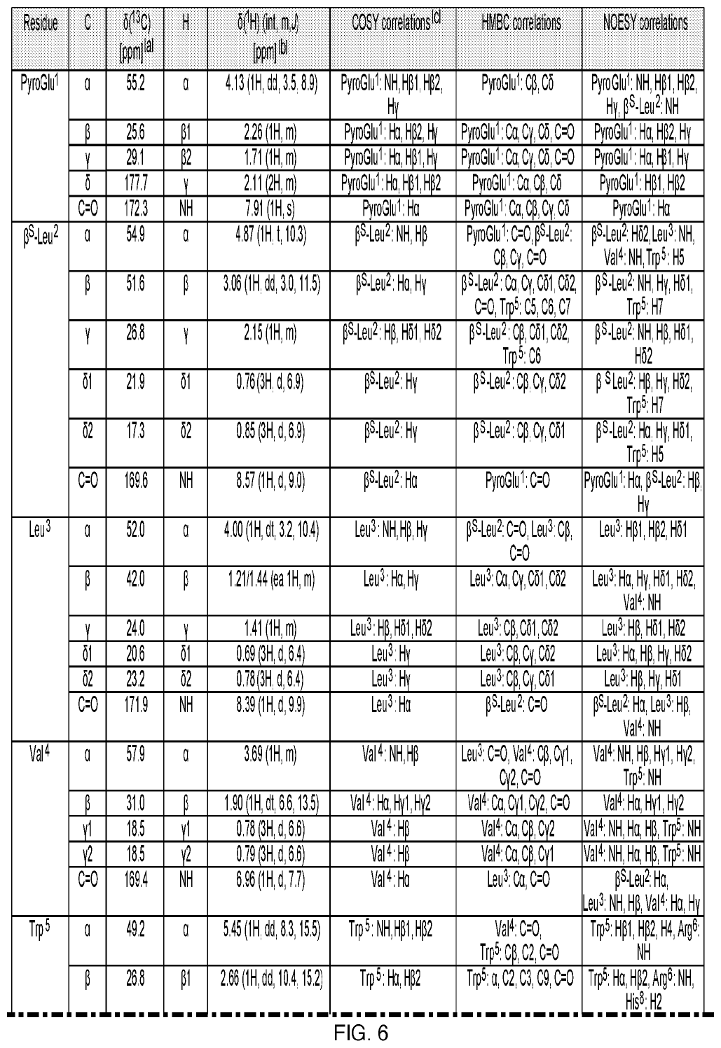

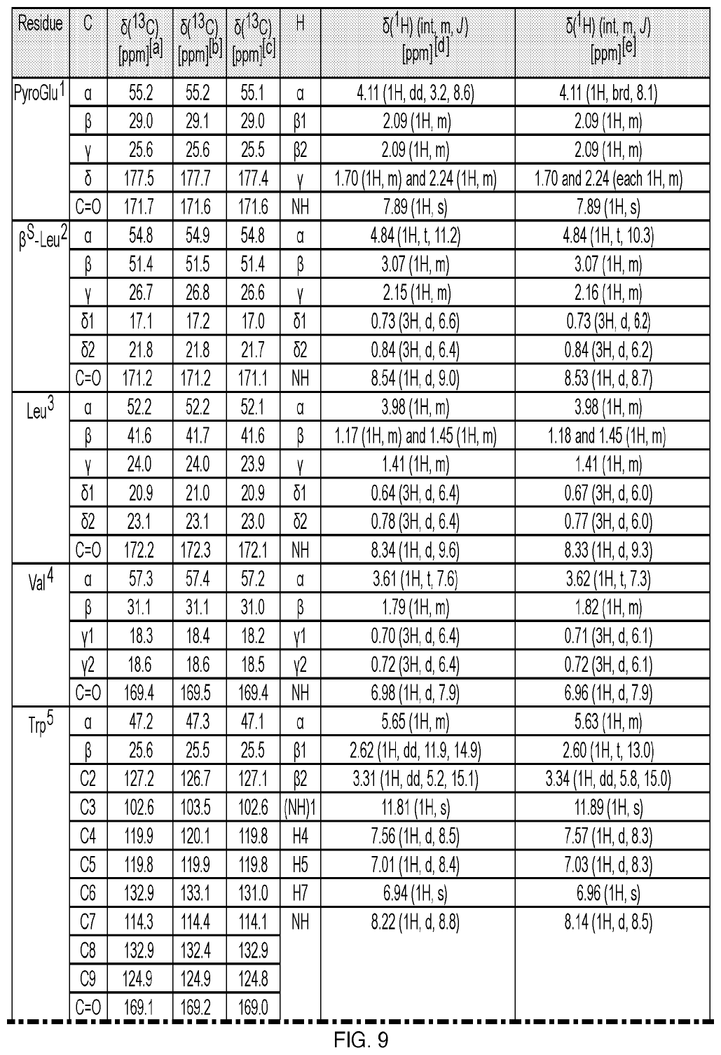

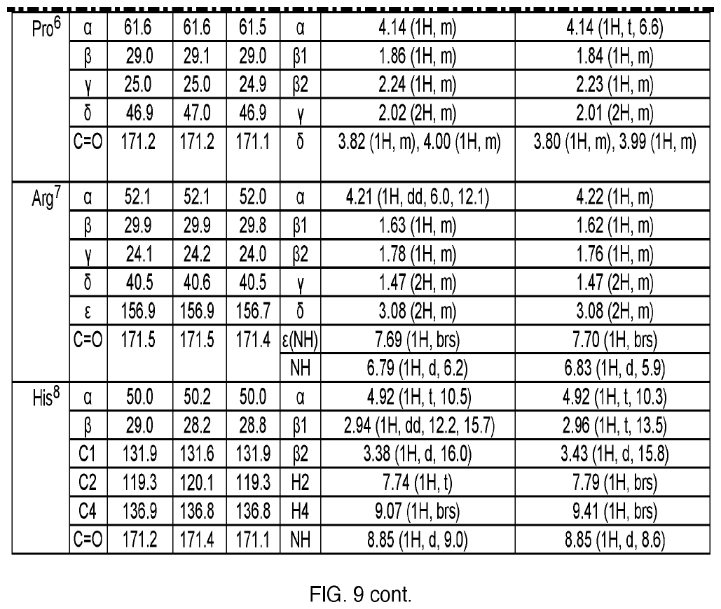

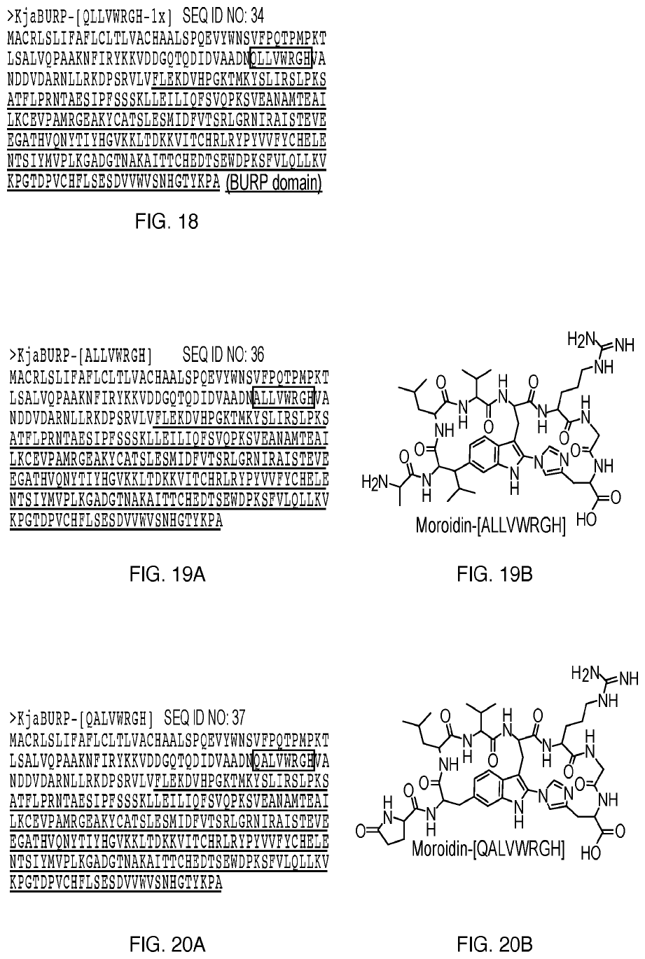

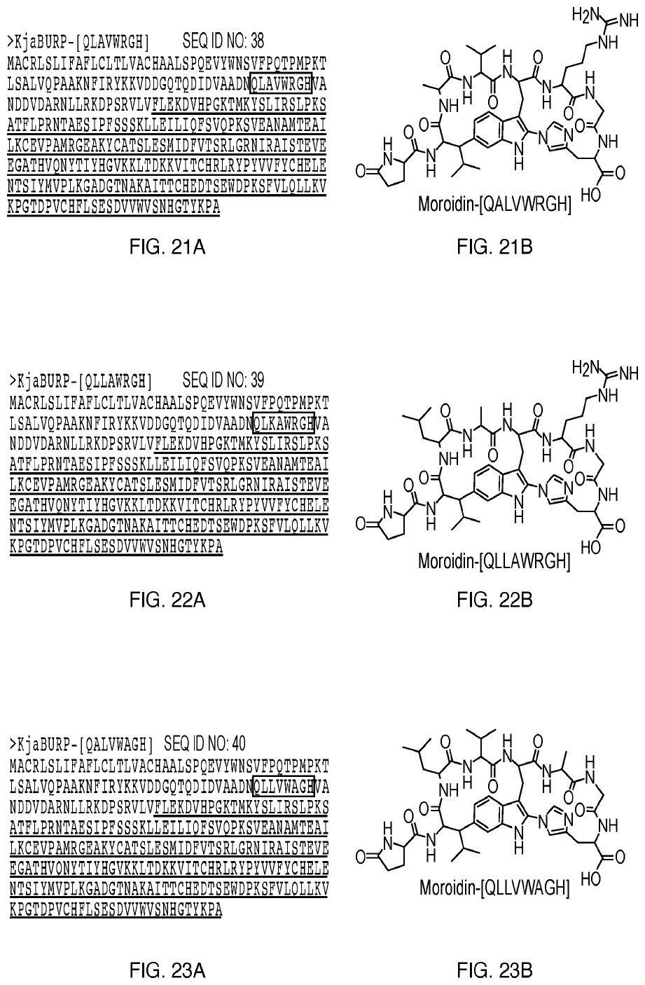

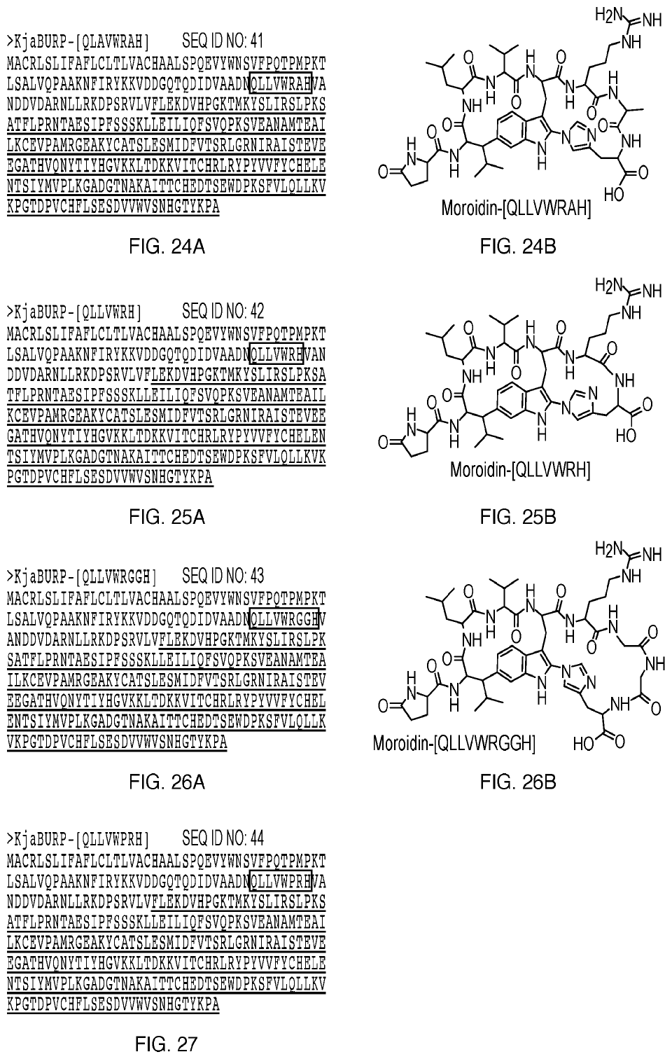

Having established a heterologous production platform of moroidin in planta, whether moroidin chemistry can be further diversified was tested. A KjaBURP construct was generated with only one moroidin core peptide in its N-terminus. Transient expression of this KjaBURP-[QLLVWRGH-lx] (SEQ ID NO: 35) construct in N. bethaminana resulted in moroidin biosynthesis (FIG. 5A and FIG. 18). Next, an alanine scanning of the moroidin core peptide of KjaBURP-[QLLVWRGH-lx] (SEQ ID NO: 35) was performed and showed that six of the eight core peptide residue positions can be mutated to alanine, while bicyclic peptide formation is maintained (FIG. 5B and FIGS. 19-24). Interestingly, leucine at the second position, which is involved in one of the macrocyclizations, appears to be mutable; however, the yield of the corresponding analyte moroidin-[QALVWRGH] (SEQ ID NO: 37) was very low. Mutation of tryptophan at the fifth position and of histidine at the eighth position resulted in abolished bicyclic peptide formation (FIG. 5B). In addition, whether the moroidin ring size can be changed via the KjaBURP system was tested. The left ring could neither be expanded nor reduced. The right ring could be reduced at least by one amino acid to yield moroidin-[QLLVWRH] (FIG. 5B and FIG. 25A-B), matching the structure of celogentin A, and expanded by at least one residue to yield moroidin-[QLLVWRGGH] (SEQ ID NO: 43) (FIG. 5B and FIG. 26A-B). Whether the potent tubulin polymerization inhibitor celogentin C can be produced via the KjaBURP tobacco expression system was tested. Transient expression of KjaBURP-[QLLVWPRH] (SEQ ID NO: 45) indeed resulted in the formation of an analyte matching the structure of celogentin C (FIG. 5B, FIG. 9, and FIG. 27). Given the defined stereochemistry of KjaBURP-derived moroidin and celogentin C, the predicted 3D structure of newly identified amaranthipeptide B from Amaranthus sp. by transient expression of KjaBURP-[QLLVWRNH] (SEQ ID NO: 46) in N. benthamiana was confirmed. Ultimately, the diversification study indicates that the moroidin biosynthetic pathway could be exploited to generate moroidin peptide libraries due to the intrinsic substrate promiscuity of the system.

Finally, whether moroidin can be produced in higher yields via heterologous precursor expression than through source plant extraction was determined. For this, moroidin abundance in peptide extracts of dried tobacco leaves after transient expression of KjaBURP-[QLLVWRGH-lx] (SEQ ID NO: 35) (one moroidin core peptide), KjaBURP (three moroidin core peptides) and KjaBURP-N (three moroidin core peptides)+KjaBURP-no-core with moroidin abundance in peptide extracts of dried C. argentea flowers were compared. LC-MS-based moroidin quantification showed that moroidin was produced at levels ten times and four times higher by transient expression of unmodified KjaBURP and KjaBURP-[QLLVWRGH-lx](SEQ ID NO: 35), respectively, than that via extraction of C. argentea flowers (FIG. 5C), suggesting that heterologous production in tobacco could serve as an alternative to source plant extraction for moroidin peptide supply.

### DISCUSSION

The discovery of moroidin peptides by searching the corresponding precursor genes in plant genomes and transcriptomes and subsequent peptide-targeted metabolomics highlights that new peptide chemistry could be discovered from the growing plant genomic and transcriptomic resources by gene-guided approaches. BURP-domain genes were used previously to identify new lyciumin chemotypes from genome-sequenced plants. Moreover, similar precursor-gene-guided approaches have proven effective for the discovery of head-to-tail-cyclic ribosomal peptides from plants. Described herein, these findings also define DUF2775 proteins as a new class of precursor peptides in plants, which enables future efforts of mining plant genomes and transcriptomes for ribosomal peptides. Interestingly, DUF2775 precursor peptides often contain multiple core peptides, which seems to be a common feature of cyanobacterial, plant and fungal ribosomal peptide biosynthesis and is not typically observed in microbial RiPP biosynthesis. It is noteworthy that the two candidate moroidin precursor genes, AhyMorA and AhyCelA, are colocalized in the A. hypochondriacus genome in a region also populated with multiple BURP-domain genes (FIG. 2A).

The present disclosure reveals the moroidins as a new class of plant ribosomal peptides, which follow a similar proposed biosynthetic logic as the previously characterized lyciumins. Moroidin biosynthesis most likely starts by posttranslational modification of the moroidin core peptide in the precursor peptide by a BURP domain to yield a core peptide with a Leu-Trp-His cross-link. The proteolytic stability of the modified core peptide enables maturation by non-specific proteases of the linear peptide sequences N- and C-terminally of the core peptide and N-terminal protection by a glutamine cyclotransferase to form the pyroglutamate moiety from glutamine. The flanking of moroidin core peptides in C. argentea precursor CarMorA with asparagines and the detection of an [Asn9]-moroidin derivative suggests that proteolytic cleavage can also occur by specific endopeptidases such as asparagine-endopeptidases, which are also involved in head-to-tail cyclic peptide biosynthesis. The in vivo experiments on KjaBURP, presented here, suggest a catalytic role of BURP domains in plant peptide biosynthesis. Although BURP-domain genes have been previously associated with plant stress responses, no biochemical activity has been reported on this protein domain to date. The BURP domain is characterized by a CH—(X)10-CH—(X)25-27-CH—(X)25-26-CH motif (SEQ ID NO: 64), where X can be any amino acid, indicating a metal-cofactor-binding site. The BURP-domain-catalyzed bicyclization in moroidin involves a C(sp3)-H functionalization at the leucine β-carbon, which most likely requires a radical enzyme mechanism such as the similar C—C bond formation during streptide biosynthesis catalyzed by a radical SAM enzyme. It is interesting to note that moroidins are derived from at least two different precursor protein families, the DUF2775 domain and the BURP domain. The detection of DUF2775-moroidin precursors in Amaranthaceae and Urticaceae and BURP-moroidin precursors in Fabaceae and Rosaceae suggests possible independent evolution of moroidin chemistry in the plant kingdom from different precursor proteins. A full elucidation of moroidin biosynthesis in the context of the growing plant genomic resources will establish a comprehensive model for moroidin evolution in the plant kingdom. In addition, the high expression of candidate moroidin precursor genes in source tissues suggests an important biological role of these bicyclic peptides in producer plants.

### Materials and Methods

### Materials and Instruments

All chemicals were purchased from Sigma-Aldrich, unless otherwise noted.

Oligonucleotide primers were purchased from Integrated DNA Technologies, Inc. Synthetic genes were purchased as gBlocks® from Integrated DNA Technologies, Inc. Solvents for liquid chromatography high-resolution mass spectrometry were Optima® LC-MS grade (Fisher Scientific) or LiChrosolv® LC-MS grade (Millipore). High-resolution mass spectrometry analysis was performed on a Thermo ESI-Q-Exactive Orbitrap MS coupled to a Thermo Ultimate 3000 UHPLC system. Low-resolution mass spectrometry analysis was done on a Thermo ESI-QQQ Quantum Access Max MS coupled to a Thermo Ultimate 3000 UHPLC system. NMR analysis was performed on a Bruker Avance II 600 MHz NMR spectrometer equipped with a High Sensitivity Prodigy Cryoprobe. Preparative and semipreparative HPLC was performed on a Shimadzu LC-20AP liquid chromatograph equipped with a SPD-20A UV/VIS detector and a FRC-10A fraction collector.

### Plant Material

Celosia argentea var. cristata seeds for cultivation were purchased from David's Garden Seeds™. Amaranthus hypochondriacus seeds for cultivation were purchased from Strictly Medicinal Seeds™. Amaranthus cruentus seeds for cultivation were purchased from SEEDVILLE USA™. Dendrocnide moroides seeds for cultivation were a gift from Marcus Schultz. Bauhinia tomentosa seeds for extraction were purchased from rarepalmseeds.com™. Kerria japonica was purchased as a mature plant from Green Promise Farms™. Nicotiana benthamiana seeds for cultivation were a gift from the Lindquist lab (Whitehead Institute, MIT).

### Plant Cultivation

C. argentea seeds, A. hypochondriacus seeds, A. cruentus seeds and D. moroides seeds were grown in SunGro® Propagation Mix soil with added vermiculite (Whittemore Inc.) and added fertilizer in a greenhouse with a 16 h light/8 h dark cycle for six months. K. japonica was grown from a mature plant in MiracleGro® potting soil as a potted plant in full sun with occasional application of organic fertilizer. N. benthamiana was grown from seeds in SunGro® Propagation Mix soil with added vermiculite (Whittemore Inc.) and added fertilizer in a greenhouse with a 16 h light/8 h dark cycle for three months.

Transcriptomic Analysis of Celosia argentea and Dendrocnide Moroides

C. argentea flower tissue and D. moroides leaf tissue were removed from three month-old plants, respectively. Total RNA was extracted from the respective plant samples with the QIAGEN RNeasy Plant Mini kit. RNA quality was assessed by Agilent Bioanalyzer. Strand-specific mRNA libraries were prepared (TruSeq Stranded Total RNA with Ribo Zero Library Preparation Kit, Illumina) and sequenced with a HiSeq2500 Illumina sequencer in HISEQRAPID mode (100×100). Illumina sequence raw-files were combined and assembled by the Trinity package (v2.4) or rnaSPAdes (v1.0, kmer 25.75). Gene expression was estimated by quantifying mapped raw sequencing reads to the de novo assembled transcriptomes using RSEM41. Candidate moroidin precursor transcripts were searched in the de novo transcriptomes by querying its predicted core peptide sequences QLLVWRGH (SEQ ID NO: 59) or ELLVWRGH by blastp algorithm on an internal Blast server. In order to clone and sequence candidate moroidin precursor genes, cDNA was prepared from C. argentea flower total RNA and D. moroides leaf total RNA, respectively, with SuperScript® III First-Strand Synthesis System (Invitrogen). Transcripts with candidate moroidin core peptides were used to design cloning primers (CarMorA-pEAQ-HT-fwd: TGCCCAAATTCGCGACCGGTATGAAGTTCTTAATCACTTCTCTCG (SEQ ID NO: 1), CarMorA-pEAQ-HT-rev: CCAGAGTTAAAGGCCTCGAGGCTAGTTAGATGTAGGCTCC (SEQ ID NO: 2) and DmoMorA-pEAQ-HT-fwd: TGCCCAAATTCGCGACCGGTATGAAGTCTTCATCTGCAATCG (SEQ ID NO: 3), DmoMorA-pEAQ-HT-rev: CCAGAGTTAAAGGCCTCGAGCTAATGACCTCTCCAAACTAAGAG (SEQ ID NO: 4)) for amplification of candidate precursor genes CarMorA and DmoMorA, respectively, with Phusion® High-Fidelity DNA polymerase (New England Biolabs). CarMorA and DmoMorA were cloned into pEAQ-HT, which was linearized by restriction enzymes AgeI and XhoI, by Gibson cloning assembly (New England Biolabs). Cloned CarMorA and DmoMorA were sequenced by Sanger sequencing from pEAQ-HT-CarMorA and pEAQ-HT-DmoMorA, respectively.

Chemotyping of Moroidin Peptides from Plant Material

For peptide chemotyping, 0.2 g plant material (fresh weight) was frozen and ground with mortar and pestle. Ground plant material was extracted with 10 mL methanol for 1 h at 37° C. in a glass vial. Plant methanol extract was dried under nitrogen gas in a separate glass vial. Dried plant methanol extract was resuspended in water (10 mL) and partitioned with hexane (2×10 mL) and ethyl acetate (2×10 mL), and subsequently extracted with n-butanol (10 mL). The n-butanol extract was dried in vacuo and resuspended in 2 mL methanol for liquid chromatography-mass spectrometry (LC-MS) analysis. Peptide extract was subjected to high resolution MS analysis with the following LC-MS parameters: LC—Phenomenex Kinetex® 2.6 m C18 reverse phase 100 A 150×3 mm LC column, LC gradient: solvent A—0.1% formic acid, solvent B—acetonitrile (0.1% formic acid), 0-2 min: 5% B, 2-22 min: 5-95% B, 22-24 min: 95% B, 24-30 min: 5% B, 0.5 mL/min, MS—positive ion mode, Full MS: Resolution 70000, mass range 450-1250 m/z, dd-MS2 (data-dependent MS/MS): resolution 17500, Loop count 5, Collision energy 15-35 eV (stepped), dynamic exclusion 0.5 s. LC-MS data was analyzed with QualBrowser in the Thermo Xcalibur software package (version 3.0.63, ThermoScientific).

For comparative quantitative chemotyping of moroidin in C. argentea flower (3 month-old plants) and N. benthamiana leaves (6 week-old plants) after transient expression of KjaBURP constructs for six days, peptides were extracted from dried plant tissues (0.1 g) as described above from three different plants of the same age. Peptide extracts were subjected to high-resolution MS analysis by full-scan MS analysis with the following LC-MS parameters: LC—Phenomenex Kinetex® 2.6 m C18 reverse phase 100 A 150×3 mm LC column, LC gradient: solvent A—0.1% formic acid, solvent B—acetonitrile (0.1% formic acid), 0-1 min: 5% B, 1-6 min: 5-95% B, 6-6.5 min: 95% B, 6.5-10 min: 5% B, MS—positive ion mode, mass range 600-1100 m/z. Moroidin ion abundance values were determined by peak area integration from each moroidin EIC chromatogram (Δm 6 ppm) in QualBrowser in the Thermo Xcalibur software package (version 3.0.63, ThermoScientific).

### Moroidin Peptide Genome Mining

Prediction of moroidin genotypes: For prediction of moroidin precursor genes in a plant genome, CarMorA homologs (GenBank: MK947386) were searched by tblastn search in 6-frame translated genome sequences (JGI Phytozome v12.1). All identified CarMorA homologs from each plant genome were then searched for moroidin core peptide sequences with the search criteria based on known moroidin structures (FIG. 10) of (1) a glutamine and leucine as the first and second amino acid, respectively, in the core peptide sequence, (2) a tryptophan at the fifth position, and (3) a histidine at the seventh or eighth position of the core peptide sequence (FIG. 10). Candidate moroidin precursor genes AhyCelA and AhyMorA identified in the genome of Amaranthus hypochondriacus (JGI Phytozome, v2.1) were verified as expressed transcripts in de novo assembled transcriptomes of A. hypochondriacus var. Plainsman. Eight transcriptome RNA-seq datasets (SRR1598909, SRR1598910, SRR1598911, SRR1598912, SRR1598913, SRR1598914, SRR1598915, SRR1598916) of genome-sequenced A. hypochondriacus were combined, assembled by Trinity (v2.4) and searched for AhyCelA and AhyMorA sequences, yielding corresponding transcripts. Furthermore, AhyCelA was verified by cloning a homolog from closely related Amaranthus cruentus. Herein, A. cruentus root tissue was removed from a three month-old plant and total RNA was extracted with the QIAGEN RNeasy Plant Mini kit. RNA quality was assessed by Agilent Bioanalyzer. A strand-specific mRNA library was prepared (TruSeq Stranded Total RNA with Ribo Zero Library Preparation Kit, Illumina) and sequenced with a HiSeq2500 Illumina sequencer in HISEQRAPID mode (100×100). Illumina sequence raw-files were combined and assembled by rnaSPAdes (v1.0, kmer 25.75). AhyCelA was searched in de novo rnaSPAdes-assembled root transcriptome of A. cruentus on an internal Blast server42 by tblastn to identify AcrCelA. In order to clone and sequence candidate moroidin precursor AcrCelA, cDNA was prepared from A. cruentus root total RNA with SuperScript® III First-Strand Synthesis System (Invitrogen). AcrCelA transcript was used to design cloning primers (AcrCelA-pEAQ-HT-fwd: TGCCCAAATTCGCGACCGGTATGAAGTTCTCTCTCATTTCTC (SEQ ID NO: 5), AcrCelA-pEAQ-HT-rev: CCAGAGTTAAAGGCCTCGAGCTAGAAACTGATGCCCTCATC (SEQ ID NO: 6)) for amplification of candidate precursor gene with Phusion® High-Fidelity DNA polymerase (New England Biolabs). AcrCelA was cloned into pEAQ-HT, which was linearized by restriction enzymes AgeI and XhoI, by Gibson cloning assembly (New England Biolabs). Cloned AcrCelA was sequenced by Sanger sequencing from pEAQ-HT-AcrCelA. Signal peptides of candidate moroidin precursor peptides were predicted by SignalP (v5.0).

Moroidin precursor gene sequences derived from genome mining of Amaranthus hypochondriacus:

AhyMorA [Amaranthus hypochondriacus]: see SEQ ID NO: 9.

AhyCelA [Amaranthus hypochondriacus]: see SEQ ID NO: 11.

Prediction of moroidin chemotypes: A moroidin structure was predicted from a putative moroidin core peptide sequence by transformation of the glutamine at the first position to a pyroglutamate and formation of a covalent bond between the indole-C6 of the tryptophan at the fifth position with the β-carbon of the leucine at the second position and a covalent bond between the indole-C2 of the tryptophan to the N1 of a C-terminal histidine-imidazole at the seventh or eighth position.

Moroidin chemotyping: LC-MS data of peptide extracts from a predicted moroidin producing plant was analyzed for moroidin mass signals by (a) parent mass search (base peak chromatogram of calculated [M+H]+ of predicted moroidin structure, Δm=5-8 ppm), and (b) iminium ion mass search of specific amino acids of a predicted structure in MS/MS data (for example, pyroglutamate iminium ion [M+H]+ 84.04439 m/z, Δm=5 ppm). Putative mass signals of predicted moroidin structures were confirmed by MS/MS data analysis with QualBrowser in the Thermo Xcalibur software package (version 3.0.63, ThermoScientific).

### Moroidin Peptide Transcriptome Mining

For moroidin transcriptome mining, transcriptomes of terrestrial plants from the 1 kp database were assembled by rnaSPAdes (v1.0, kmer 25.75 or, if failed, default kmer 55) (FIG. 8). See FIG. 8 for a list of successful and failed de novo assemblies. De novo assembled transcriptomes were searched for CarMorA homologs by tblastn search on an internal Blast server. Candidate moroidin precursors were predicted with the same core peptide search criteria as for moroidin genome mining with some precursors being partial sequences due to failed complete de novo assembly (FIGS. 3A-C, FIG. 8 and FIG. 15).

Cloning of Candidate Moroidin Precursor KjaBURP from Kerria japonica

Candidate moroidin precursor KjaBURP was identified as a partial transcript in a de novo-rnaSPAdes assembly of a Kerria japonica transcriptome (NCBI SRA: ERR2040423). In order to clone a complete sequence of KjaBURP, a de novo leaf transcriptome of Kerria japonica was generated. Total RNA was extracted from leaves of a two year-old K. japonica plant with the QIAGEN RNeasy Plant Mini kit. RNA quality was assessed by Agilent Bioanalyzer. A strand-specific mRNA library was prepared (TruSeq Stranded Total RNA with Ribo Zero Library Preparation Kit, Illumina) and sequenced with a HiSeq2500 Illumina sequencer in HISEQRAPID mode (100×100). Illumina sequence raw-files were combined and assembled by rnaSPAdes (v1.0, kmer 25.75). KjaBURP transcripts in the de novo leaf transcriptome of K. japonica enabled the design of cloning primers (KjaBURP-pEAQ-HT-fwd: TGCCCAAATTCGCGACCGGTATGGCGTGCCGTCTCTCAC (SEQ ID NO: 13), KjaBURP-pEAQ-HT-rev: CCAGAGTTAAAGGCCTCGAGTTATGCAGGTTTATATGTGCCATGG (SEQ ID NO: 14)) for amplification of candidate precursor gene KjaBURP with Phusion® High-Fidelity DNA polymerase (New England Biolabs). KjaBURP was cloned into pEAQ-HT, which was linearized by restriction enzymes AgeI and XhoI, by Gibson cloning assembly (New England Biolabs). Cloned KjaBURP was sequenced by Sanger sequencing from pEAQ-HT-KjaBURP. For KjaBURP co-expression analysis of its core peptide domain and its BURP domain, one gene construct, KjaBURP-no-core, was synthesized as an IDT gBlock®, and one gene construct, KjaBURP-N, was cloned from K. japonica cDNA (see FIG. 16). KjaBURP-no-core was cloned into pEAQ-HT using cloning PCR primers KjaBURP-pEAQ-HT-fwd and KjaBURP-pEAQ-HT-rev as described above. KjaBURP-N was cloned into pEAQ-HT using cloning primers KjaBURP-pEAQ-HT-fwd and KjaBURP-N-rev (CCAGAGTTAAAGGCCTCGAGTTACTCCAAGAAGACAAGTACTCGGG) as described above.

Cloned gene construct of KjaBURP-N for transient (co-)expression in N. benthamiana was as follows:

KjaBURP-N: see SEQ ID NO: 15.

Synthetic gene construct of KjaBURP-no-core for transient (co-)expression in N. benthamiana was as follows:

KjaBURP-no-core: see SEQ ID NO: 16.

Heterologous Expression of Moroidin Precursor Genes in Nicotiana benthamiana
Agrobacterium tumefaciens LBA4404 was transformed with pEAQ-HT-AcrCelA, pEAQ-HTDmoMorA, pEAQ-HT-CarMorA, pEAQ-HT-KjaBURP or pEAQ-HT-KjaBURP-mutants by electroporation (2.5 kV), plated on YM agar (0.4 g yeast extract, 10 g mannitol, 0.1 g sodium chloride, 0.2 g magnesium sulfate (heptahydrate), 0.5 g potassium phosphate, (dibasic, trihydrate), 15 g agar, ad 1 L Milli-Q Millipore water, adjusted pH 7) with 100 μg/mL rifampicin, 50 μg/mL kanamycin and 100 μg/mL streptomycin and incubated for two days at 30° C. A 5 mL starter culture of YM medium with 100 μg/mL rifampicin, 50 μg/mL kanamycin and 100 μg/mL streptomycin was inoculated with a clone of Agrobacterium tumefaciens LBA4404 pEAQ-HTKjaBURP (or other precursor gene) and incubated for 24-36 h at 30° C. on a shaker at 225 rpm. Subsequently, the starter culture was used to inoculate a 25 mL culture of YM medium with 100 μg/mL rifampicin, 50 μg/mL kanamycin and 100 μg/mL streptomycin, which was incubated for 24 h at 30° C. on a shaker at 225 rpm. The cells from the 25 mL culture were centrifuged for 30 min at 3000 g, the YM medium was discarded and cells were resuspended in MMA medium (10 mM MES KOH buffer (pH 5.6), 10 mM magnesium chloride, 100 μM acetosyringone) to give a final optical density of 0.8. The Agrobacterium suspension was infiltrated into the bottom of leaves of Nicotiana benthamiana plants (six week old). N. benthamiana plants were placed in the shade two hours before infiltration. After infiltration, N. benthamiana plants were grown as described above for six days. Subsequently, infiltrated leaves were collected and subjected to peptide chemotyping. For co-expression of KjaBURP-N and KjaBURP-no-core, a 1:1 suspension mixture of A. tumefaciens LBA4404 pEAQ-HT-KjaBURP-N and A. tumefaciens LBA4404 pEAQ-HTKjaBURP-no-core at OD 0.8 was infiltrated into N. benthamiana leaves.
Moroidin Diversification Via Transient Expression of KjaBURP Mutants in Nicotiana benthamiana.

KjaBURP mutants were synthesized as gBlocks® and cloned into pEAQ-HT for heterologous expression in N. benthamiana as described above. Chemotyping of infiltrated N. benthamiana leaves for moroidins was done as described above.

### Purification and Structure Elucidation of Moroidin Peptides.

For moroidin and [Asn9]-moroidin isolation, Celosia argentea flowers (1 kg fresh weight) were ground with a cryogenic tissue grinder and extracted for 16 h with methanol shaking at 225 rpm and 37° C. For celogentin C isolation, N. benthamiana leaves after transient expression of KjaBURP-[QLLVWPRH] (SEQ ID NO: 45) for six days (2.5 kg fresh weight) were ground with a cryogenic tissue grinder and extracted for 16 h with methanol shaking at 225 rpm and 37° C. Methanol extracts were filtered and dried in vacuo. Dried methanol extracts were resuspended in water and partitioned twice with hexane and twice with ethyl acetate and then extracted twice with n-butanol. n-butanol extracts were dried in vacuo. Dried n-butanol extracts were resuspended in 10% methanol and separated by flash column liquid chromatography with Sephadex LH20 as a stationary phase and 10% methanol as a mobile phase. Fractions were collected with a fraction collector and analyzed for moroidin peptide content by low resolution-LC-MS with the following LC-MS settings: LC—Phenomenex Kinetex® 2.6 m C18 reverse phase 100 A 150×3 mm LC column, LC gradient: solvent A—0.1% formic acid, solvent B—acetonitrile (0.1% formic acid), 0.5 mL/min, 0-1 min: 5% B, 1-8 min: 5-95% B, 8-10 min: 95% B, 10-15 min: 5% B, MS—positive ion mode, Full MS: moroidin—950-1000 m/z, [Asn9]-moroidin—1075-1125 m/z, celogentin C—1000-1050 m/z. LH20 fractions with moroidins were combined, dried in vacuo, resuspended in 10% acetonitrile (0.1% trifluoroacetic acid) and subjected to preparative HPLC with a Phenomenex Kinetex® 5 m C18 reverse phase 100 A 150×21.2 mm LC column as a stationary phase. LC settings were as follows: solvent A—0.1% trifluoroacetic acid, solvent B—acetonitrile (0.1% trifluoroacetic acid), 7.5 mL/min, moroidin and [Asn9]-moroidin—0-3 min: 10% B, 3-43 min: 10-40% B, 43-45 min: 40-95% B, 45-48 min: 95% B, 48-49 min: 95-10% B, 49-69 min: 10% B, Celogentin C—1.LC: 0-3 min: 10% B, 3-43 min: 10-50% B, 43-45 min: 50-95% B, 45-48 min: 95% B, 48-49 min: 95-10% B, 49-69 min: 10% B, 2.LC: 0-3 min: 20% B, 3-43 min: 20-35% B, 43-45 min: 35-95% B, 45-48 min: 95% B, 48-49 min: 95-30% B, 49-69 min: 20% B. Preparative HPLC fractions with Moroidin/Premoroidin or celogentin C, respectively, were combined, dried in vacuo, resuspended in 20% acetonitrile (0.1% trifluoroacetic acid) and subjected to semipreparative HPLC with a Phenomenex Kinetex® 5 m C18 reverse phase 100 A 250×10 mm LC column as a stationary phase. LC settings were as follows: Solvent A—0.1% trifluoroacetic acid, solvent B—acetonitrile (0.1% trifluoroacetic acid), 1.5 mL/min, moroidin (20 mg), [Asn9]-moroidin (5 mg)—0-2 min 10% B, 2-5 min 10-32% B, 5-30 min 32-37% B, 30-32 min 37-95% B, 32-36 min 95% B, 36-60 min 10% B, and celogentin C (13 mg)—0-5 min 25% B, 5-17.5 min 25-30% B, 17.5-19.5 min 30-95% B, 19.5-20 min 95% B, 20-20.5 min 95-25% B, 20.5-40 min 25% B. For NMR analysis, moroidin, [Asn9]-moroidin and celogentin C were each dissolved in DMSO-d6 and analyzed for ¹H NMR, ¹³C NMR, ¹H-¹H-DFQ-COSY, ¹H-¹H-TOCSY, HSQC, HMBC and NOESY data. NMR data was analyzed with TopSpin software (v3.5 and v4.0) from Bruker.

Synthetic gene constructs for moroidin diversification experiments by transient expression in N. benthamiana are as follows:
KjaBURP-[QLLVWRGH-lx]: see SEQ ID NO: 35
KjaBURP-[QLLVWPRH]: see SEQ ID NO: 45
KjaBURP-[QLLVWRNH]: see SEQ ID NO: 46
KjaBURP-[ALLVWRGH]: see SEQ ID NO: 47
KjaBURP-[QALVWRGH]: see SEQ ID NO: 48
KjaBURP-[QLAVWRGH]: see SEQ ID NO: 49
KjaBURP-[QLLAWRGH]: see SEQ ID NO: 50
KjaBURP-[QLLVARGH]: see SEQ ID NO: 51
KjaBURP-[QLLVWAGH]: see SEQ ID NO: 52
KjaBURP-[QLLVWRAH]: see SEQ ID NO: 53
KjaBURP-[QLLVWRGA]: see SEQ ID NO: 54
KjaBURP-[QLLVWRGGH]: see SEQ ID NO: 55
KjaBURP-[QLLVGWRGH]: see SEQ ID NO: 56
KjaBURP-[QLVWRGH]: see SEQ ID NO: 57
KjaBURP-[QLLVWRH]: see SEQ ID NO: 58

### Accession Numbers

Gene sequences generated in this study (GenBank): CarMorA (MK947386), DmoMorA (MK947387), AcrCelA (MK947388), KjaBURP (MK947389).

Transcriptomes generated in this study (NCBI SRA): C. argentea flower (SRR9095475), D. moroides leaf (SRR9112680), A. cruentus root (SRR9095301), K. japonica leaf (SRR9095474).

LCMS datasets (MassIVE): C. argentea flower (MSV000083812), D. moroides leaf (MSV000083814), A. cruentus root (MSV000083810), A. cruentus seed (MSV000083809), A. cruentus flower (MSV000083808), A. hypochondriacus seed (MSV000083811), K. japonica leaf (MSV000083815), B. tomentosa seed (MSV000083813).

MS/MS spectra (GNPS)39: moroidin (CCMSLIB00005435900), [Asn9]-moroidin (CCMSLIB00005435901), [Ala9]-moroidin (CCMSLIB00005435919), [Ala9-Ala10]-moroidin (CCMSLIB00005435920), celogentin C (CCMSLIB00005435902), amaranthipeptide A (CCMSLIB00005435903), amaranthipeptide B (CCMSLIB00005435904), moroidin-[QLLVWRAH] (CCMSLIB00005435905) (SEQ ID NO: 41), moroidin-[QLLVWRSH](CCMSLIB00005435906), [AsnO-Gln1]-moroidin (CCMSLIB00005435912), [Gln1]-moroidin (CCMSLIB00005435912), [Gln1]-moroidin-[QLLVWRAH] (CCMSLIB00005435915) (SEQ ID NO: 41), [Gln1-Val9]-moroidin (CCMSLIB00005435916), [Gln1-Val9]-moroidin-[QLLVWRAH] (CCMSLIB00005435917) (SEQ ID NO: 41), [Val9]-moroidin (CCMSLIB00005435914), [Val9]-moroidin-[QLLVWRAH] (CCMSLIB00005435918) (SEQ ID NO: 41), moroidin-[ALLVWRGH] (CCMSLIB00005435907) (SEQ ID NO: 36), moroidin-[QALVWRGH] (CCMSLIB00005435908) (SEQ ID NO: 37), moroidin-[QLAVWRGH](CCMSLIB00005435909) (SEQ ID NO: 38), moroidin-[QLLAWRGH](CCMSLIB00005435910) (SEQ ID NO: 39), moroidin-[QLLVWAGH](CCMSLIB00005435911) (SEQ ID NO: 40), moroidin-[QLLVWRH](CCMSLIB00005435921) (SEQ ID NO: 42), moroidin-[QLLVWRGGH](CCMSLIB00005435922) (SEQ ID NO: 43).

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of producing one or more moroidin cyclic peptides, the method comprising:
a) providing a host cell comprising a transgene encoding a moroidin precursor peptide, or a biologically-active fragment thereof, wherein the moroidin precursor peptide, or biologically-active fragment thereof, comprises one or more core moroidin peptide domains;
b) expressing the transgene in the host cell to thereby produce a moroidin precursor peptide, or biologically-active fragment thereof,
wherein the moroidin precursor peptide, or biologically-active fragment thereof, is converted to one or more moroidin cyclic peptides in the host cell; or
wherein the moroidin precursor peptide, or biologically-active fragment thereof, is isolated from the host cell and is then converted into a moroidin cyclic peptide in vitro using one or more enzymes, optionally wherein the one or more enzymes are an enzyme that cyclizes the moroidin precursor peptide, an endopeptidase, a glutamine cyclotransferase, an exopeptidases, or a combination thereof.

2. The method of claim 1, wherein the transgene is operably linked to a heterologous promoter in the host cell.

3. The method of claim 1, wherein the transgene is introduced in a vector.

4. The method of claim 1, further comprising introducing the transgene into the host cell.

5. The method of claim 4, further comprising introducing a vector comprising the transgene into the host cell.

6. The method of claim 1, wherein the moroidin precursor peptide comprises a plurality of core moroidin peptide domains.

7. The method of claim 6, wherein the core moroidin peptide domains encode two or more different moroidin cyclic peptides.

8. The method of claim 1, wherein the host cell expresses one or more enzymes that cyclize the moroidin precursor peptide; one or more endopeptidases; one or more glutamine cyclotransferases; and one or more exopeptidases, or a combination thereof, optionally wherein the host cell naturally expresses one or more of the enzymes that cyclize the moroidin precursor peptide, the one or more endopeptidases, the one or more glutamine cyclotransferases; and/or the one or more exopeptidases, and/or wherein the host cell is genetically engineered to stably or transiently express one or more of the enzymes that cyclize the moroidin precursor peptide, the one or more endopeptidases, the one or more glutamine cyclotransferases; and/or the one or more exopeptidases, optionally so that the cell expresses all of the enzymes needed to produce the moroidin cyclic peptide.

9. The method of claim 1, wherein asparagine is immediately N-terminal to the core moroidin peptide domain.

10. The method of claim 9, wherein the endopeptidase is an asparagine endopeptidase.

11. The method of claim 1, wherein asparagine, alanine, or valine is immediately C-terminal to the core moroidin peptide domain.

12. The method of claim 1, wherein the host cell is a bacterial or archael cell, a fungal cell (optionally a yeast cell), an insect cell, a mammalian cell, or a plant cell, optionally wherein the plant cell is a cultured plant cell or is in a plant.

13. The method of claim 12, wherein the plant cell is an Amaranthaceae family plant cell.

14. The method of claim 13, wherein the plant cell is an Amaranthus genus plant cell.

15. The method of claim 14, wherein the plant cell is an Amaranthus hypochondriacus plant cell or an Amaranthus cruentus plant cell.

16. The method of claim 13, wherein the plant cell is a Beta genus plant cell.

17. The method of claim 16, wherein the plant cell is a Beta vulgaris plant cell.

18. The method of claim 13, wherein the plant cell is a Chenopodium genus plant cell.

19. The method of claim 18, wherein the plant cell is a Chenopodium quinoa plant cell.

20. The method of claim 12, wherein the plant cell is a Fabaceae family plant cell.

21. The method of claim 20, wherein the plant cell is a Glycine genus plant cell.

22. The method of claim 21, wherein the plant cell is a Glycine max plant cell.

23. The method of claim 20, wherein the plant cell is a Medicago genus plant cell.

24. The method of claim 23, wherein the plant cell is a Medicago truncatula plant cell.

25. The method of claim 12, wherein the plant cell is a Solanaceae family plant cell.

26. The method of claim 25, wherein the plant cell is a Solanum genus plant cell.

27. The method of claim 26, wherein the plant cell is a Solanum melongena plant cell.

28. The method of claim 26, wherein the plant cell is a Solanum tuberosum plant cell.

29. The method of claim 25, wherein the plant cell is a Nicotiana genus plant cell.

30. The method of claim 29, wherein the plant cell is a Nicotiana benthamiana plant cell.

31. The method of claim 25, wherein the plant cell is a Capsicum genus plant cell.

32. The method of claim 31, wherein the plant cell is a Capsicum annuum plant cell.

33. The method of claim 1, wherein the moroidin precursor peptide comprises a moroidin precursor peptide from Dendrocnide moroides, Celosia argentea, Amaranthus hypochondriacus, Kerria japonica, or a species indicated in FIG. 8 as harboring a predicted core peptide of moroidin precursor homolog.

34. The method of claim 1, wherein the moroidin precursor peptide comprises one or more DUF2775-domains.

35. The method of claim 1, wherein: (i) each core moroidin peptide domain comprises the sequence QL(X)₂W(X)₁₋₂H, wherein X is any amino acid, optionally wherein the sequence comprises QLLVWRGH (SEQ ID NO: 59); or (ii) wherein at least one core moroidin peptide domain comprises a variant of the sequence QL(X)₂W(X)₁₋₂H, wherein X is any amino acid, optionally wherein the W and/or the H is not mutated.

36. A method of generating a library of nucleic acids encoding moroidin precursor peptides, or biologically-active fragments thereof, the method comprising constructing a plurality of vectors, each vector comprising a nucleic acid encoding a different moroidin precursor peptide, or biologically-active fragment thereof, operably linked to a heterologous promoter for expression in a host cell.

37. The method of claim 36, further comprising introducing the plurality of vectors into host cells, wherein the moroidin precursor peptide, or biologically-active fragments thereof, is converted to one or more moroidin cyclic peptides in the host cell.

38. The method of claim 37, wherein the host cell is a plant cell.

39. The method of claim 38, wherein the plant cell is a Solanaceae family plant cell.

40. The method of claim 39, wherein the plant cell is a Nicotiana genus plant cell.

41. The method of claim 40, wherein the plant cell is a Nicotiana benthamiana plant cell.

42. The method of claim 37, further comprising isolating a moroidin cyclic peptide from the host cell.

43. The method of claim 37, further comprising assaying for an activity of interest either in crude extract from the host cell or a moroidin peptide isolated from the host cell.

44. The method of claim 37, further comprising introducing a nucleic acid encoding a moroidin peptide having an activity of interest into a second cell, optionally wherein the second cell is a bacterial or archael cell, a fungal cell (e.g., a yeast cell), an insect cell, a mammalian cell, or a plant cell, optionally wherein the plant cell is a cultured plant cell or is in a plant.

45. The method of claim 44, wherein the second cell is a plant cell, optionally wherein the plant cell is a cultured plant cell or is in a plant.

46. The method of claim 45, wherein the plant cell is an Amaranthaceae family plant cell.

47. The method of claim 46, wherein the plant cell is an Amaranthus genus plant cell.

48. The method of claim 47, wherein the plant cell is an Amaranthus hypochondriacus plant cell.

49. The method of claim 46, wherein the plant cell is a Beta genus plant cell.

50. The method of claim 49, wherein the plant cell is a Beta vulgaris plant cell.

51. The method of claim 46, wherein the plant cell is a Chenopodium genus plant cell.

52. The method of claim 51, wherein the plant cell is a Chenopodium quinoa plant cell.

53. The method of claim 45, wherein the plant cell is a Fabaceae family plant cell.

54. The method of claim 53, wherein the plant cell is a Glycine genus plant cell.

55. The method of claim 54, wherein the plant cell is a Glycine max plant cell.

56. The method of claim 53, wherein the plant cell is a Medicago genus plant cell.

57. The method of claim 56, wherein the plant cell is a Medicago truncatula plant cell.

58. The method of claim 45, wherein the plant cell is a Solanaceae family plant cell.

59. The method of claim 58, wherein the plant cell is a Solanum genus plant cell.

60. The method of claim 59, wherein the plant cell is a Solanum melongena plant cell.

61. The method of claim 59, wherein the plant cell is a Solanum tuberosum plant cell.

62. The method of claim 58, wherein the plant cell is a Nicotiana genus plant cell.

63. The method of claim 62, wherein the plant cell is a Nicotiana benthamiana plant cell.

64. The method of claim 58, wherein the plant cell is a Capsicum genus plant cell.

65. The method of claim 64, wherein the plant cell is a Capsicum annuum plant cell.

66. An isolated nucleic acid comprising a nucleotide sequence encoding a moroidin precursor peptide, or a biologically-active fragment thereof, operably linked to a heterologous promoter.

67. The isolated nucleic acid of claim 66, wherein the moroidin precursor peptide comprises a plurality of core moroidin peptide domains.

68. The isolated nucleic acid of claim 67, wherein the core moroidin peptide domains encode two or more different moroidin cyclic peptides.

69. The isolated nucleic acid of claim 66, wherein the moroidin precursor peptide comprises a moroidin precursor peptide from Dendrocnide moroides, Celosia argentea, Amaranthus hypochondriacus, Kerria japonica, or a species indicated in FIG. 8 as harboring a predicted core peptide of moroidin precursor homolog and/or wherein the moroidin precursor peptide, or a biologically-active fragment thereof comprises one or more core moroidin peptide domains and: (i) each core moroidin peptide domain comprises the sequence QL(X)₂W(X)₁₋₂H, wherein X is any amino acid, optionally wherein the sequence comprises QLLVWRGH (SEQ ID NO: 59); or (ii) wherein at least one core moroidin peptide domain comprises a variant of the sequence QL(X)₂W(X)₁₋₂H, wherein X is any amino acid, optionally wherein the W and/or the H is not mutated.

70. The isolated nucleic acid of claim 66, wherein the moroidin precursor peptide comprises one or more DUF2775-domains.

71. The isolated nucleic acid of claim 66, wherein the nucleic acid is a cDNA.

72. A vector comprising the nucleic acid of claim 66.

73. A host cell comprising the nucleic acid of claim 66 or the vector of claim 72.

74. The host cell of claim 73, wherein the host cell is a bacterial or archael cell, a fungal cell (e.g., a yeast cell), an insect cell, a mammalian cell, or a plant cell, optionally wherein the plant cell is a cultured plant cell or is in a plant.

75. The host cell of claim 74, wherein the plant cell is an Amaranthaceae family plant cell.

76. The host cell of claim 75, wherein the plant cell is an Amaranthus genus plant cell.

77. The host cell of claim 76, wherein the plant cell is an Amaranthus hypochondriacus plant cell.

78. The host cell of claim 75, wherein the plant cell is a Beta genus plant cell.

79. The host cell of claim 78, wherein the plant cell is a Beta vulgaris plant cell.

80. The host cell of claim 75, wherein the plant cell is a Chenopodium genus plant cell.

81. The host cell of claim 80, wherein the plant cell is a Chenopodium quinoa plant cell.

82. The host cell of claim 74, wherein the plant cell is a Fabaceae family plant cell.

83. The host cell of claim 82, wherein the plant cell is a Glycine genus plant cell.

84. The host cell of claim 83, wherein the plant cell is a Glycine max plant cell.

85. The host cell of claim 82, wherein the plant cell is a Medicago genus plant cell.

86. The host cell of claim 85, wherein the plant cell is a Medicago truncatula plant cell.

87. The host cell of claim 74, wherein the plant cell is a Solanaceae family plant cell.

88. The host cell of claim 87, wherein the plant cell is a Solanum genus plant cell.

89. The host cell of claim 88, wherein the plant cell is a Solanum melongena plant cell.

90. The host cell of claim 88, wherein the plant cell is a Solanum tuberosum plant cell.

91. The host cell of claim 87, wherein the plant cell is a Nicotiana genus plant cell.

92. The host cell of claim 91, wherein the plant cell is a Nicotiana benthamiana plant cell.

93. The host cell of claim 87, wherein the plant cell is a Capsicum genus plant cell.

94. The host cell of claim 93, wherein the plant cell is a Capsicum annuum plant cell.

95. A library comprising a plurality of nucleic acid molecules, each nucleic acid molecule comprising a nucleotide sequence encoding a moroidin precursor peptide, or a biologically-active fragment thereof.

96. The library of claim 95, wherein the nucleotide sequence encoding a moroidin precursor peptide, or a biologically-active fragment thereof, is operably linked to a heterologous promoter in each nucleic acid molecule.

97. The library of claim 95 or 96, wherein the nucleic acid molecules are cDNA molecules.

98. A moroidin cyclic peptide produced by the method of any one of claims 1-65.

99. A method of producing one or more moroidin cyclic peptides, the method comprising:
a) providing a host cell comprising a transgene encoding a polypeptide that comprises one or more core moroidin peptide domains; and
b) expressing the transgene in the host cell to thereby produce a polypeptide that comprises one or more core moroidin peptide domains.

100. The method of claim 99, wherein the polypeptide is converted to one or more moroidin cyclic peptides in the host cell, or wherein the polypeptide is isolated from the cell and converted to one or more moroidin cyclic peptides outside the cell, optionally by using one or more enzymes, optionally wherein the one or more enzymes are an enzyme that cyclizes the moroidin precursor peptide, an endopeptidases, a glutamine cyclotransferase, an exopeptidases, or a combination thereof.

101. A method of characterizing a moroidin cyclic peptide of claim 1, the method comprising contacting the moroidin cyclic peptide with a mammalian cell and measuring one or more biological activities of the moroidin cyclic peptide, optionally wherein measuring comprises measuring the ability of the moroidin cyclic peptide to inhibit mitosis of the cell, optionally wherein the cell is a cancer cell and/or is a human cell and/or comprises measuring the ability of the moroidin cyclic peptide to inhibit tubulin polymerization.

102. The method of claim 101, wherein the contacting is in vitro.

103. The method of claim 101, wherein the contacting comprises administering the moroidin cyclic peptide to a mammalian subject, optionally wherein the subject is human.

104. The method of claim 101, wherein the method comprising contacting a plurality of different moroidin cyclic peptides with mammalian cells and identifying a moroidin cyclic peptide with anti-mitotic activity equal to or greater than that of moroidin or of a celogentin, optionally wherein the celogentin is selected from any one of celogentin A, B, C, D, E, F, G, H, I, J, or K.

105. A method of inhibiting mitosis in a cell, optionally wherein the cell is a mammalian cell, the method comprising contacting the cell with a moroidin cyclic peptide of any of claims 1-104, optionally wherein the cell is a cancer cell and/or is a human cell.

106. The method of claim 105, wherein the contacting is in vitro.

107. The method of claim 105, wherein the contacting comprises administering the moroidin cyclic peptide to a subject, optionally wherein the subject is a mammalian subject, optionally wherein the subject is human and/or the subject has cancer.

108. A method of treating cancer comprising administering the moroidin cyclic peptide of any of claims 1-104 to a mammalian subject in need thereof, optionally wherein the subject is a human.

109. The method of claim 108, further comprising administering a second anti-cancer agent to the subject.

110. A pharmaceutical composition comprising a moroidin cyclic peptide of or produced according to the method of any one of claims 1 through 65.


SEQUENCE LISTING (66 sequences)

SEQ ID NO: 1 - DNA, 45 bp, synthetic construct
  misc_feature 1..45: note = CarMorA-pEAQ-HT-fwd primer
  source 1..45: mol_type = other DNA
tgcccaaattcgcgaccggtatgaagttcttaatcacttctctcg

SEQ ID NO: 2 - DNA, 40 bp, synthetic construct
  misc_feature 1..40: note = CarMorA-pEAQ-HT-rev primer
  source 1..40: mol_type = other DNA
ccagagttaaaggcctcgaggctagttagatgtaggctcc

SEQ ID NO: 3 - DNA, 42 bp, synthetic construct
  misc_feature 1..42: note = DmoMorA-pEAQ-HT-fwd primer
  source 1..42: mol_type = other DNA
tgcccaaattcgcgaccggtatgaagtcttcatctgcaatcg

SEQ ID NO: 4 - DNA, 44 bp, synthetic construct
  misc_feature 1..44: note = DmoMorA-pEAQ-HT-rev primer
  source 1..44: mol_type = other DNA
ccagagttaaaggcctcgagctaatgacctctccaaactaagag

SEQ ID NO: 5 - DNA, 42 bp, synthetic construct
  misc_feature 1..42: note = AcrCelA-pEAQ-HT-fwd primer
  source 1..42: mol_type = other DNA
tgcccaaattcgcgaccggtatgaagttctctctcatttctc

SEQ ID NO: 6 - DNA, 41 bp, synthetic construct
  misc_feature 1..41: note = AcrCelA-pEAQ-HT-rev primer
  source 1..41: mol_type = other DNA
ccagagttaaaggcctcgagctagaaactgatgccctcatc

SEQ ID NO: 7 - protein, 262 aa, Celosia argentea
MKFLITSLVFALVLLVGTTEGRKDPGEYWGAKAVEALVDLSLKGEEGSLVNAFEPRPNQLLVWRGHNRVVPTESNGETSSESFVKDFEYRPNQLLVWRGHNRVVPKESNGETSSESFLTDFEPRPNQLLVWRGHNRDVPKESNGETSSESFVKDFEPRPNQLLVWRGHNRVVSKESNGEISSESFVKDFEPIPNQLLVWRGHNKVVPKESNGETLSESFLKDFEPRPNQLLVWRGHRKANPTENDGATTNAPFVKDLEPTSN

SEQ ID NO: 8 - protein, 134 aa, Dendrocnide moroides
MKSSSAIVGALLLLLLHSWGTAEARKDPGTNFQSVVKEKDQTLPEAIRDLFVSKKKWGDNLELEPFQLLVWRGHAADKPAEATEDIPAETDQQNIVIKKEETMPEAIKDLFVSNKKWGENIELEPFQLLVWRGH

SEQ ID NO: 9 - DNA, 222 bp, Amaranthus hypochondriacus
  source 1..222: mol_type = genomic DNA
atgaagttctttctcatttctcttgcttttacacttctcttgcttatcaacatatctgaaggacgaaaagatccgggatattattggggaaagaaagcagttgaggctcttgttgatgaaaatttgagtgctgatgagaagttcattgtcatcaacaattttgagcctaggcctaaccaacttctcgtatggcgtgggcacaataagattgcttcaacataa

SEQ ID NO: 10 - protein, 73 aa, Amaranthus hypochondriacus
MKFFLISLAFTLLLLINISEGRKDPGYYWGKKAVEALVDENLSADEKFIVINNFEPRPNQLLVWRGHNKIAST

SEQ ID NO: 11 - DNA, 981 bp, Amaranthus hypochondriacus
  source 1..981: mol_type = genomic DNA
atgaagttctctctcatttctctagcttttacacttctcttgttcatcaatataagtgaaggacgaaaagatccgggagactattggggaaagaaatcaattgaggctcttattgattcaaatttgagtgaagatgaaaagtttattgtcatcaacaactttgaaccaaggcctaatcaacttcttatatggcctagacatgatacggtttctccaacagagaccaaagaggcatcagtttacaataaacatgtgttgaataactttgaaccgagacccaaccaacttcttgtatggcctagacataacaaggttgttccttccaatactaatgaggcctcaacctctagtgaatctctcgtgaacaactttgagcctagacctaaccaacttctcgtatggcctagacacaataaggtttctcctacagagaataaggaggtatcaatatccaataaacaagtggtgaataactttgagcctagacctaaccaacttcttgtttggccaagacataataaggttgctccttctgatactaatgaggtctcaacctctagtgaatctgtcgtaaacaactttgagcctagacctaaccaacttctcatatggcctagacacaataaggttgctccaacagaaactagcgaggcatcagtttccaatgaatttgttgtaaacaactttgagcccaggcctaaccaacttcttgtatggcctagacataacaaggttgtttcttcagatactaatgaggcatcaacttctagtgaatctatcgtgaacaactttgaacctaggcctaaccaacttcttgtatggcgtaatcacaataaggttagtcctaatgaaattaatgacgtaacaatttctagtgaatctcttgcaatcaactttgagcctaggcctaaccagcttctcgtatggcgtaatcacaataaggttgttcctaaagaaactgatgagggcatcagtttctag

SEQ ID NO: 12 - protein, 326 aa, Amaranthus hypochondriacus
MKFSLISLAFTLLLFINISEGRKDPGDYWGKKSIEALIDSNLSEDEKFIVINNFEPRPNQLLIWPRHDTVSPTETKEASVYNKHVLNNFEPRPNQLLVWPRHNKVVPSNTNEASTSSESLVNNFEPRPNQLLVWPRHNKVSPTENKEVSISNKQVVNNFEPRPNQLLVWPRHNKVAPSDTNEVSTSSESVVNNFEPRPNQLLIWPRHNKVAPTETSEASVSNEFVVNNFEPRPNQLLVWPRHNKVVSSDTNEASTSSESIVNNFEPRPNQLLVWRNHNKVSPNEINDVTISSESLAINFEPRPNQLLVWRNHNKVVPKETDEGISF

SEQ ID NO: 13 - DNA, 39 bp, synthetic construct
  misc_feature 1..39: note = KjaBURP-pEAQ-HT-fwd primer
  source 1..39: mol_type = other DNA
tgcccaaattcgcgaccggtatggcgtgccgtctctcac

SEQ ID NO: 14 - DNA, 45 bp, synthetic construct
  misc_feature 1..45: note = KjaBURP-pEAQ-HT-rev primer
  source 1..45: mol_type = other DNA
ccagagttaaaggcctcgagttatgcaggtttatatgtgccatgg

SEQ ID NO: 15 - DNA, 492 bp, Kerria japonica
  source 1..492: mol_type = genomic DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttggagaggccacgtcgccattgatgatgatgcagccgctgataatcaacttcttgtttggagaggccatgtcgccattgatgatgatgatgctgctgctgataatcaacttcttgtttggagaggccacgtcgccattcatgatgatgctgccgctgataatcaacttcttgtttggagagcacacgtcgccaatgatgatgttgatgctcgcaatctactccgtaaagacccatcccgagtacttgtcttcttggag

SEQ ID NO: 16 - DNA, 945 bp, Kerria japonica
  source 1..945: mol_type = genomic DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 17 - protein, 314 aa, Kerria japonica
MACRLSLIFAFLCLTLVACHAALSPQEVYWNSVFPQTPMPKTLSALVQPAAKNFIRYKKVDDGQTQDIDVAADNVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 18 - protein, 300 aa, Bauhinia tomentosa
PNSQLLVWRSHATDDQLRDDPNSQLLVWRSHATDDQLRDDPNSQLLVWFGRHATDDQLRDDPNSQLLVWRSHFKDQPAKGDNTAKFLFLNENDLHPGKKLEMLFTQSPYSKALMSREVADKIPFSLEKLPEILKMFSVKPESKEAQTIKKGIHHCEMPSNVKEEEKHCVTSLESMVDYITSKLGTNVKAYSTEVERETGEYTTYTIAPGVKKLSDDAMACHQKHYPYVLFNCHKVGNSNTVYFVPLEGSDGSRVKAVVVCHLNTLDWDAKHIAFQQLGIKPGAPVCHFSPADNVVWTPTN

SEQ ID NO: 19 - protein, 8 aa, synthetic construct
  REGION 1..8: note = Moroidin-[QLLVWRAH] Predicted Core Peptide
QLLVWRAH

SEQ ID NO: 20 - protein, 8 aa, synthetic construct
  REGION 1..8: note = Moroidin-[QLLVWRSH] Predicted Core Peptide
QLLVWRSH

SEQ ID NO: 21 - protein, 69 aa, Celosia argentea
SFVKDFEYRPNQLLVWRGHNRVVPTESNGETSSESFLTDFEPRPNQLLVWRGHNRDVPKESNGETSSES

SEQ ID NO: 22 - protein, 262 aa, Celosia argentea
MKFLITSLVFALVLLVGTTEGRKDPGEYWGAKAVEALVDLSLKGEEGSLVNAFEPRPNQLLVWRGHNRVVPTESNGETSSESFVKDFEYRPNQLLVWRGHNRVVPKESNGETSSESFVTDFEPRPNQLLVWRGHNRAVPKERNGETSSESFVKDFEPRPNQLLVWRGHNRVVSKESNGEISSESFVKDFEPIPNQLLVWRGHNKVVPKESNGETLSESFLKDFEPRPNQLLVWRGHRKANPTENDGATTNAPFVKDLEPTSN

SEQ ID NO: 23 - protein, 134 aa, Dendrocnide moroides; identical to SEQ ID NO: 8

SEQ ID NO: 24 - protein, 103 aa, Dendrocnide moroides
FQSVVREKDQTLPEAIRDLFVSKKKWGDNLELEPFQLLVWRGHAADKPAEATEDIPAETDQQNIVIKKEETMPEAIKDLFVSNKKWGENIELEPFQLLVWRGH

SEQ ID NO: 25 - DNA, 3493 bp, Amaranthus hypochondriacus
  source 1..3493: mol_type = genomic DNA
atgaagttctctctcatttctctagcttttacacttctcttggtaagcatcaccatttattttattttttatttatatgagtttgtttttaatttctgtaattaccagaaagatatctatgattgatcggaaactattttcttttaaaattttttagttatttcaaatatatttaaaggaattgatgaataattaaagtgatatttaaaagtggaaaaaatgtatgaatgagatgaaaagtaagttatatgcgtaaataaaaattgaaaaaaaaaagagtgtatcatgactaaaaatacgaatgaagataaatttaatagaacaaattaaaaaaatggatatcatggattttgcaataaaagacatcgaaatgaaggatagggttggtaagtaagttgattagcggatttaaatattgaaaattgaaaaagaggtaaactattataaatataaatatataatactacctcctattcaacttaaatgtcctatttttattttggaactattcactcatcattcttaatttgtattttactcttaatctataagttaaaacatagtcatgtgggatcttgtttgattcgtctcaatacaaggattattaatatcaattttttataatttttaattaagaataatttgagatattaagggttaaaatgttgtctaggcaagtgtgaaaaattaaatgggacatttaggccgaataagagggagtagtacacaactttgaaactcattggtattgttgttcttgttgttgatgtggttgttatcaatgtatctattgttttgatttataataataataataataataataataataataatataataataataatataataataataataataataataataataataataattattattattattattattattatttttattattattattattattattattattattattattattattattattatccttttgttcattaatgaagtttccacaaaaattgttcttaggaattaagaaaaaaataaaatctttagatagcggataaattattttaatgaataacaaatttgatgggagaaaatttggtactataaacattaacaaatatttttaaaaattagtgtaagaaattattattatcctttcgttccttaatgaagtttccacaaaaattgttcttaggaattaagaaaaaaataaaatcttttagatagcggataaattattttaatgaataaaaaatttgatgggagaaaatctataactattactaaaacaaaatactgctggcatcatcaccttttaaaatcacttacgtgtcacctcctcattaaaaattttacctctaaaatgcaaagatgacatcattgttattatgattatctttgactttattatttttaaattttgagtataataattcatattgattattttttactttttgattcacattatttaatgtattaaaaaaatatatactatatttcatttatttcaattataaaatctttttaattttatttattaaaggggtattatttagtgtattggatattttgagatgtaaaatttatttcttataatttcaatttctgaatttatataaatatgttaaaaattttgataaaaccgtgcatcgcacgtgaactatctagtttggtactataaacattaacaaatatttttaaaaattagtataagaaaaaataaggacaaaaactaataagaaatatttttataaagttactggaaaacatgtggcaacaccaagaaatataaaagtattaaaatatagtttgtggaagatacgtggggactataagtattattaaaatactagtataaaaactcgtgcattgcacgaaaattttaatatgattagatatatataaaaataatttgctctcaataaaattcttaaaaagtacaatcgtaataatcagattaattaaaccacttgtttttaatgttaagtaaaatttaataaatgtataacacattatatattttaaaccttattaatatcattcgatgcaacaaaaaattttaaattattagttttataaaaatattaaaaaattttagtttgtaaagatattaaatttaaggtgtgtgaacatacatttaaaaaattttaacttattagttttataaaaatattaaaaatatttagtttgtaaagatagtaaatttaaggtgtgtgaacatacatttaaaaaactcaatgtttttaatgttaagttacatgaggctaacacatggaatttttcagttcttttattaaattgtatgattaaaatgacattgaacaatgttatttttgtccaaactttgtgatataaatagaaatattctttatatggcaaatgagactttctttaaggaacagaaggtgcaattattattattataaaattaatgtggttttacttatatgaatatgttgtgtacatctctgcatacattatttaagtagaaatttgcatgattttaatctgaatgaatttatattatggtgcatatgtcctaatatattgtgttttgtgtagttcatcaatataagtgaaggacgaaaagatccgggagactattggggaaagaaatcaattgaggctcttattgattcaaatttgagtgaagatgaaaagtttattgtcatcaacaactttgaaccaaggcctaatcaacttcttatatggcctagacatgatacggtttctccaacagagaccaaagaggcatcagtttacaataaacatgtgttgaataactttgaaccgagacccaaccaacttcttgtatggcctagacataacaaggttgttccttccaatactaatgaggcctcaacctctagtgaatctctcgtgaacaactttgagcctagacctaaccaacttctcgtatggcctagacacaataaggtttctcctacagagaataaggaggtatcaatatccaataaacaagtggtgaataactttgagcctagacctaaccaacttcttgtttggccaagacataataaggttgctccttctgatactaatgaggtctcaacctctagtgaatctgtcgtaaacaactttgagcctagacctaaccaacttctcatatggcctagacacaataaggttgctccaacagaaactagcgaggcatcagtttccaatgaatttgttgtaaacaactttgagcccaggcctaaccaacttcttgtatggcctagacataacaaggttgtttcttcagatactaatgaggcatcaacttctagtgaatctatcgtgaacaactttgaacctaggcctaaccaacttcttgtatggcgtaatcacaataaggttagtcctaatgaaattaatgacgtaacaatttctagtgaatctcttgcaatcaactttgagcctaggcctaaccagcttctcgtatggcgtaatcacaataaggttgttcctaaagaaactgatgagggcatcagtttctag

SEQ ID NO: 26 - DNA, 2528 bp, Amaranthus hypochondriacus
  source 1..2528: mol_type = genomic DNA
atgaagttctttctcatttctcttgcttttacacttctcttggtaagcatcaccatttatttacttctccactttttttttaacttttatctatttatataagtttgcatttaatttctataattaccagaaattaatctatgctcagagataaatataaataaatcatttatttaaaaatatttttaagaaattgatgaataaaatgatattttatagaaaagtgggtgaaatatgtaaatgagatgaaaaaataagttaagaattaaataagtcatggacaaaaaaaaaatgtgaacaaatttaatgaaaattaaattttgtggaacagaaaaagaaattaaaatacttttaattgaatttgcaatacaaagacatcgaaatgattgacgatgttgaaaagtaagttaattagcgaaaacggaagtccatttataatacttcctttgttgcataatactccttacatataaatagtaatacagttcatctttaattttatttcggtaatttcgaatcatgcataaaaaataatataatcatatgaaattttattttattttccataaaatcaagttttttagtatgtaattttcataatttttcgtttagataaaattaaaatattaaatatcaaaatatacatttaattacatacaaaaagaaatgtatgtattgggtaattttgaaatactcaactctctaaattctcatcggcatccagcattctcaattgcaaagcatttatggagagattgataatggaggtggcgaacggaagggttaagttgttgatatggttgttaattaataaatattattatttttgttgaagtattcatgtgttacccatatgtgagttgttccacatcggagaaagagaagagtatgcatcactttataaactcgagtaactcctattatcaccaattggttttagtagtgaaccttctttgggtttatgagtggactacttcttcccctccttgtttgggttacccagacctattttctagttatcatggtatcagagccgaaggtgataataaaaataatatttctattaaatattacacatattttcaaataagcccatcccatggtgagactatctctattagactggtttaatgtatacttaatattttaaattgattatttataactttaatgtgatcacttataatcttaaaatgagttgacggtgttaaaagggaatcacttttatgaaaaaacattgggagaacaatggtttttaaccaattccctttcttcactaaaattgcttggtaaaaagctcaaaaaaaaaatctctttctcttataaaatctcaataaaaatcaaaagaagtttagatcagatattacttggtgttgagacatctagctaagaattttcatgtcattggtactttggtcaatgaagaagcaatgtgagcctaaatgatgaagaagcaatgtttagtaaaaaaatgacttagctaactgattccattattaagactagggccgaacaaagtttggtctaaaccgtaaacaaaaccgtaatttgaatatttggtctgatctacggtctaaatggtctggtctgattttttttcaaaaaaaaatacggtttacagtctggtcccgattttttaatttttcagaccagaccagaccataaaccgtactttgaccaaaaaccatattttttttataaaaatcgggtttctacctagatattttgagatgttattatttttatatagtaatatatacttgaataatgttgatggagttaactaataacaaattatatttatttagtgtttgtcagtgcgaaatattttcatagaacatatattaattaatttggaaataaatataaaataaaaaccgtaaaccagaccagaccaaaccgttgtagaaaggtttggtccggtccgatctgaaacttttccatatcggaatttttggtttggtctgattttagtgtcagaccagaccaaaccggaccgtgtgcacccctaattaagactctaaattttgcatacttaacgcacaatcaatacttgtaatcttaaaacactgatcacttacaattttaagtgatcaattagaaaaatagactaattatatgggctcgtcttgagatggtctcatacagaacttgctattattattattattattattattattgttattaattattataaattattaacaaaataatatggttttacttagtttaccaatatgttgtgtacctgtgcatacatgatttcagtagaaatttgaataaataaaatcttaaatgattttatatgttggtgcatatgtcataatatattgtgttttgtgcagcttatcaacatatctgaaggacgaaaagatccgggatattattggggaaagaaagcagttgaggctcttgttgatgaaaatttgagtgctgatgagaagttcattgtcatcaacaattttgagcctaggcctaaccaacttctcgtatggcgtgggcacaataagattgcttcaacataa

SEQ ID NO: 27 - protein, 326 aa, Amaranthus cruentus
MKFSLISLAFTLLLFINISEGRKDPGDYWGKKSIEALIDSNLSEDEKFIVINNFEPRPNQLLIWPRHDKVSPTETKEASVYNKHVLNNFEPRPNQLLVWPRHNKVVPSNTNEASTSSESLVNNFEPRPNQLLVWPRHNKVSPTENKEVSISNKQVVNNFEPRPNQLLVWPRHNKVAPSDTNEVSTSSESVVNNFEPRPNQLLIWPRHNKVAPTETSEASVSNEFVVNNFEPRPNQLLVWPRHNKVVSSDTNEASTSSESIVNNFEPRPNQLLVWRNHNKVSPNEINDVTISSESLAINFEPRPNQLLVWRNHNKVVPKETDEGISF

SEQ ID NO: 28 - protein, 126 aa, Crossopetalum rhacoma
MRNMFAFIALLSLLLSITTNAARTSPGEYWQSIMKDQPMPETIRARVFSSFKPKDFDFDTEIKPSQLLVWRNHPSRENEKIGKSEVDLSGKDFDFDAEIKPSQLLVWRNHPSRTNEKIGKSGVDLC

SEQ ID NO: 29 - protein, 300 aa, Bauhinia tomentosa; identical to SEQ ID NO: 18

SEQ ID NO: 30 - protein, 183 aa, Amaranthus tricolor
MKFFLISLAFTLLLLINISEGRKDLGDYWGKKAVEALVDENLSADEKFIVINNFEPRPNQLLVWRGHNKIAPTETNEASISNENLVNNFEPRPNQLLVWRGHNKIAPTKTNEASMSNENLVNNFEPRPNQLLVWRGHNNIAQIKTNQASSSNENLVNNFEPQPNQLLFWHRHNKVVPTETNKA

SEQ ID NO: 31 - protein, 126 aa, Amaranthus retroflexus
MKFFLISLAFTLLLLINISEGRKDPGDYWGKKAVEALVDENLSADEKFIVINNFEPKPNQLLVWRGHNKIAPTETKEASISNENLVNNFEPRPNQLLVWPRHKKVVPTETNEASVSSESVVINFEP

SEQ ID NO: 32 - protein, 164 aa, synthetic construct
  REGION 1..164: note = KjaBURP-N
MACRLSLIFAFLCLTLVACHAALSPQEVYWNSVFPQTPMPKTLSALVQPAAKNFIRYKKVDDGQTQDIDVAADNQLLVWRGHVAIDDDAAADNQLLVWRGHVAIDDDDAAADNQLLVWRGHVAIHDDAAADNQLLVWRAHVANDDVDARNLLRKDPSRVLVFLE

SEQ ID NO: 33 - protein, 314 aa, synthetic construct; identical to SEQ ID NO: 17
  REGION 1..314: note = KjaBURP-no-core

SEQ ID NO: 34 - protein, 322 aa, synthetic construct
  REGION 1..322: note = KjaBURP-[QLLVWRGH-1x]
MACRLSLIFAFLCLTLVACHAALSPQEVYWNSVFPQTPMPKTLSALVQPAAKNFIRYKKVDDGQTQDIDVAADNQLLVWRGHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 35 - DNA, 969 bp, synthetic construct
  misc_feature 1..969: note = KjaBURP-[QLLVWRGH-1x]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttggagaggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 36 - protein, 322 aa, synthetic construct
  REGION 1..322: note = KjaBURP-[ALLVWRGH]
MACRLSLIFAFLCLTLVACHAALSPQEVYWNSVFPQTPMPKTLSALVQPAAKNFIRYKKVDDGQTQDIDVAADNALLVWRGHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 37 - protein, 322 aa, synthetic construct
  REGION 1..322: note = KjaBURP-[QALVWRGH]
MACRLSLIFAFLCLTLVACHAALSPQEVYWNSVFPQTPMPKTLSALVQPAAKNFIRYKKVDDGQTQDIDVAADNQALVWRGHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 38 - protein, 322 aa, synthetic construct
  REGION 1..322: note = KjaBURP-[QLAVWRGH]
MACRLSLIFAFLCLTLVACHAALSPQEVYWNSVFPQTPMPKTLSALVQPAAKNFIRYKKVDDGQTQDIDVAADNQLAVWRGHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 39 - protein, 322 aa, synthetic construct; identical to SEQ ID NO: 38
  REGION 1..322: note = KjaBURP-[QLAVWRGH]

SEQ ID NO: 40 - protein, 322 aa, synthetic construct
  REGION 1..322: note = KjaBURP-[QLLVWAGH]
MACRLSLIFAFLCLTLVACHAALSPQEVYWNSVFPQTPMPKTLSALVQPAAKNFIRYKKVDDGQTQDIDVAADNQLLVWAGHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 41 - protein, 322 aa, synthetic construct
  REGION 1..322: note = KjaBURP-[QLLVWRAH]
MACRLSLIFAFLCLTLVACHAALSPQEVYWNSVFPQTPMPKTLSALVQPAAKNFIRYKKVDDGQTQDIDVAADNQLLVWRAHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 42 - protein, 321 aa, synthetic construct
  REGION 1..321: note = KjaBURP-[QLLVWRH]
MACRLSLIFAFLCLTLVACHAALSPQEVYWNSVFPQTPMPKTLSALVQPAAKNFIRYKKVDDGQTQDIDVAADNQLLVWRHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 43 - protein, 323 aa, synthetic construct
  REGION 1..323: note = KjaBURP-[QLLVWRGGH]
MACRLSLIFAFLCLTLVACHAALSPQEVYWNSVFPQTPMPKTLSALVQPAAKNFIRYKKVDDGQTQDIDVAADNQLLVWRGGHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 44 - protein, 322 aa, synthetic construct
  REGION 1..322: note = KjaBURP-[QLLVWPRH]
MACRLSLIFAFLCLTLVACHAALSPQEVYWNSVFPQTPMPKTLSALVQPAAKNFIRYKKVDDGQTQDIDVAADNQLLVWPRHVANDDVDARNLLRKDPSRVLVFLEKDVHPGKTMKYSLIRSLPKSATFLPRNTAESIPFSSSKLLEILIQFSVQPKSVEANAMTEAILKCEVPAMRGEAKYCATSLESMIDFVTSRLGRNIRAISTEVEEGATHVQNYTIYHGVKKLTDKKVITCHRLRYPYVVFYCHELENTSIYMVPLKGADGTNAKAITTCHEDTSEWDPKSFVLQLLKVKPGTDPVCHFLSESDVVWVSNHGTYKPA

SEQ ID NO: 45 - DNA, 969 bp, synthetic construct
  misc_feature 1..969: note = KjaBURP-[QLLVWPRH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttggcctcgacacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 46 - DNA, 969 bp, synthetic construct
  misc_feature 1..969: note = KjaBURP-[QLLVWRNH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttggcgaaaccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 47 - DNA, 969 bp, synthetic construct
  misc_feature 1..969: note = KjaBURP-[ALLVWRGH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatgcccttcttgtttggagaggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 48 - DNA, 969 bp, synthetic construct
  misc_feature 1..969: note = KjaBURP-[QALVWRGH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaagcccttgtttggagaggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 49 - DNA, 969 bp, synthetic construct
  misc_feature 1..969: note = KjaBURP-[QLAVWRGH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttgccgtttggagaggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 50 - DNA, 969 bp, synthetic construct
  misc_feature 1..969: note = KjaBURP-[QLLAWRGH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgcctggagaggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 51 - DNA, 969 bp, synthetic construct
  misc_feature 1..969: note = KjaBURP-[QLLVARGH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgttgccagaggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 52 - DNA, 969 bp, synthetic construct
  misc_feature 1..969: note = KjaBURP-[QLLVWAGH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttgggccggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 53 - DNA, 969 bp, synthetic construct
  misc_feature 1..969: note = KjaBURP-[QLLVWRAH]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttggagagcccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 54 - DNA, 969 bp, synthetic construct
  misc_feature 1..969: note = KjaBURP-[QLLVWRGA]
  source 1..969: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttggagaggcgccgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 55 - DNA, 972 bp, synthetic construct
  misc_feature 1..972: note = KjaBURP-[QLLVWRGGH]
  source 1..972: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttggagaggcggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 56 - DNA, 972 bp, synthetic construct
  misc_feature 1..972: note = KjaBURP-[QLLVGWRGH]
  source 1..972: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgttggctggagaggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 57 - DNA, 966 bp, synthetic construct
  misc_feature 1..966: note = KjaBURP-[QLVWRGH]
  source 1..966: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttgtttggagaggccacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 58 - DNA, 966 bp, synthetic construct
  misc_feature 1..966: note = KjaBURP-[QLLVWRH]
  source 1..966: mol_type = other DNA
atggcgtgccgtctctcactaatatttgcttttctctgtctcacactggtggcttgccatgctgctttatcacctcaagaagtttactggaattctgtcttcccacagacaccgatgccaaaaactctcagtgcacttgtccaacctgcagccaagaactttatccgttacaagaaagttgatgatggccaaacccaagatattgatgttgccgctgataatcaacttcttgtttggagacacgtcgccaatgatgatgttgatgctcgcaatctactccgtaaagacccatcccgagtacttgtcttcttggagaaggacgtgcatccaggaaaaacaatgaaatatagcttaatcagaagtttacctaaatccgcaacttttctgccccgcaatactgcagaatctatccccttctcatctagcaaattgttagaaattctcattcaattttcagtgcagccaaagtctgttgaagccaatgcaatgacggaagccattctaaaatgtgaagttcccgccatgagaggagaagcaaaatattgtgccacctctttggagtcaatgattgacttcgtcacttccaggcttggaagaaatattcgagcaatctcgacagaggtggaagaaggagccacccatgtgcagaattatacaatatatcatggagtgaagaagctgacagataaaaaagttattacgtgtcatcgattgaggtatccatatgttgtcttctactgccatgaattggaaaatacaagtatttatatggtgcctctgaaaggtgctgacgggacaaatgctaaagcaataacaacctgccatgaagacacatctgaatgggacccaaagagttttgtcttgcaacttctcaaagttaaaccaggaaccgaccctgtgtgccattttctttctgagagcgacgttgtttgggtttcaaaccatggcacatataaacctgcataa

SEQ ID NO: 59 - protein, 8 aa, synthetic construct
  REGION 1..8: note = Moroidin-[QLLVWRGH] Predicted Core Peptide
QLLVWRGH

SEQ ID NO: 60 - protein, 8 aa, synthetic construct
  REGION 1..8: note = Moroidin-[QLLVWPRH] Predicted Core Peptide
QLLVWPRH

SEQ ID NO: 61 - protein, 8 aa, synthetic construct
  REGION 1..8: note = Moroidin-[QLLIWPRH] Predicted Core Peptide
QLLIWPRH

SEQ ID NO: 62 - protein, 8 aa, synthetic construct
  REGION 1..8: note = Moroidin-[QLLVWRNH] Predicted Core Peptide
QLLVWRNH

SEQ ID NO: 63 - protein, 6 aa, synthetic construct
  REGION 1..6: note = core moroidin peptide domain
  VARIANT 3: note = (X)2, where X is any amino acid
  VARIANT 5: note = (X)1-2, where X is any amino acid
QLXWXH

SEQ ID NO: 64 - protein, 11 aa, synthetic construct
  REGION 1..11: note = four repeated cysteine-histidine motifs
  VARIANT 3: note = (x)10, where X can be any amino acid
  VARIANT 6: note = (x)25-27, where X can be any amino acid
  VARIANT 9: note = (x)25-26, where X can be any amino acid
CHXCHXCHXCH

SEQ ID NO: 65 - protein, 7 aa, synthetic construct
  REGION 1..7: note = first seven amino acids of the DUF2775 domain
  SITE 3: note = misc_feature - Xaa can be any naturally occurring amino acid
  SITE 5: note = misc_feature - Xaa can be any naturally occurring amino acid
KDXYXGW

SEQ ID NO: 66 - protein, 8 aa, synthetic construct
  REGION 1..8: note = Moroidin-[QLLVWRNH] Predicted Core Peptide
QLLVWRNH